SEQUENCE LISTING (680 sequences)

SEQ ID NO: 1 - DNA, 1216 bp, Homo sapiens
ggctctgtctttttagcagacgaaaaccactttggtagtgccagtgtgactcatccacaatgatttctccagtgctcatcttgttctcgagttttctctgccatgttgctattgcaggacggacctgtcccaagccagatgatttaccattttccacagtggtcccgttaaaaacattctatgagccaggagaagagattacgtattcctgcaagccgggctatgtgtcccgaggagggatgagaaagtttatctgccctctcacaggactgtggcccatcaacactctgaaatgtacacccagagtatgtccttttgctggaatcttagaaaatggagccgtacgctatacgacttttgaatatcccaacacgatcagtttttcttgtaacactgggttttatctgaatggcgctgattctgccaagtgcactgaggaaggaaaatggagcccggagcttcctgtctgtgctcccatcatctgccctccaccatccatacctacgtttgcaacacttcgtgtttataagccatcagctggaaacaattccctctatcgggacacagcagtttttgaatgtttgccacaacatgcgatgtttggaaatgatacaattacctgcacgacacatggaaattggactaaattaccagaatgcagggaagtaaaatgcccattcccatcaagaccagacaatggatttgtgaactatcctgcaaaaccaacactttattacaaggataaagccacatttggctgccatgatggatattctctggatggcccggaagaaatagaatgtaccaaactgggaaactggtctgccatgccaagttgtaaagcatcttgtaaagtacctgtgaaaaaagccactgtggtgtaccaaggagagagagtaaagattcaggaaaaatttaagaatggaatgctacatggtgataaagtttctttcttctgcaaaaataaggaaaagaagtgtagctatacagaggatgctcagtgtatagatggcactatcgaagtccccaaatgcttcaaggaacacagttctctggctttttggaaaactgatgcatccgatgtaaagccatgctaaggtggttttcagattccacacaaaatgtcacacttgtttcttgttcatccaaggaacctaattgaaatttaaaaataaagctactgaatttattgccgcacccaaaaaaaaaaaaaaaa

SEQ ID NO: 2 - DNA, 13 bp, Artificial Sequence
  Synthetic oligonucleotide
gagcactggagaa

SEQ ID NO: 3 - DNA, 13 bp, Artificial Sequence
  Synthetic oligonucleotide
tgagcactggaga

SEQ ID NO: 4 - DNA, 13 bp, Artificial Sequence
  Synthetic oligonucleotide
agatgagcactgg

SEQ ID NO: 5 - DNA, 13 bp, Artificial Sequence
  Synthetic oligonucleotide
agcaacatggcag

SEQ ID NO: 6 - DNA, 13 bp, Artificial Sequence
  Synthetic oligonucleotide
tagcaacatggca

SEQ ID NO: 7 - DNA, 13 bp, Artificial Sequence
  Synthetic oligonucleotide
atagcaacatggc

SEQ ID NO: 8 - DNA, 13 bp, Artificial Sequence
  Synthetic oligonucleotide
caatagcaacatg

SEQ ID NO: 9 - DNA, 13 bp, Artificial Sequence
  Synthetic oligonucleotide
gcaatagcaacat

SEQ ID NO: 10 - DNA, 13 bp, Artificial Sequence
  Synthetic oligonucleotide
cctgcaatagcaa

SEQ ID NO: 11 - DNA, 13 bp, Artificial Sequence
  Synthetic oligonucleotide
tcctgcaatagca

SEQ ID NO: 12 - DNA, 13 bp, Artificial Sequence
  Synthetic oligonucleotide
gtcctgcaatagc

SEQ ID NO: 13 - DNA, 13 bp, Artificial Sequence
  Synthetic oligonucleotide
tccgtcctgcaat

SEQ ID NO: 14 - DNA, 13 bp, Artificial Sequence
  Synthetic oligonucleotide
gtccgtcctgcaa

SEQ ID NO: 15 - DNA, 13 bp, Artificial Sequence
  Synthetic oligonucleotide
ggtccgtcctgca

SEQ ID NO: 16 - DNA, 13 bp, Artificial Sequence
  Synthetic oligonucleotide
aggtccgtcctgc

SEQ ID NO: 17 - DNA, 13 bp, Artificial Sequence
  Synthetic oligonucleotide
caggtccgtcctg

SEQ ID NO: 18 - DNA, 13 bp, Artificial Sequence
  Synthetic oligonucleotide
tgggacaggtccg

SEQ ID NO: 19 - DNA, 13 bp, Artificial Sequence
  Synthetic oligonucleotide
ttgggacaggtcc

SEQ ID NO: 20 - DNA, 13 bp, Artificial Sequence
  Synthetic oligonucleotide
ggcttgggacagg

SEQ ID NO: 21 - DNA, 13 bp, Artificial Sequence
  Synthetic oligonucleotide
tggcttgggacag

SEQ ID NO: 22 - DNA, 13 bp, Artificial Sequence
  Synthetic oligonucleotide
ctggcttgggaca

SEQ ID NO: 23 - DNA, 13 bp, Artificial Sequence
  Synthetic oligonucleotide
tctggcttgggac

SEQ ID NO: 24 - DNA, 13 bp, Artificial Sequence
  Synthetic oligonucleotide
catctggcttggg

SEQ ID NO: 25 - DNA, 13 bp, Artificial Sequence
  Synthetic oligonucleotide
tcatctggcttgg

SEQ ID NO: 26 - DNA, 13 bp, Artificial Sequence
  Synthetic oligonucleotide
atcatctggcttg

SEQ ID NO: 27 - DNA, 13 bp, Artificial Sequence
  Synthetic oligonucleotide
aatcatctggctt

SEQ ID NO: 28 - DNA, 13 bp, Artificial Sequence
  Synthetic oligonucleotide
aaatcatctggct

SEQ ID NO: 29 - DNA, 13 bp, Artificial Sequence
  Synthetic oligonucleotide
tcctggctcatag

SEQ ID NO: 30 - DNA, 13 bp, Artificial Sequence
  Synthetic oligonucleotide
ctcctggctcata

SEQ ID NO: 31 - DNA, 13 bp, Artificial Sequence
  Synthetic oligonucleotide
tctcctggctcat

SEQ ID NO: 32 - DNA, 13 bp, Artificial Sequence
  Synthetic oligonucleotide
ccggcttgcagga

SEQ ID NO: 33 - DNA, 13 bp, Artificial Sequence
  Synthetic oligonucleotide
cccggcttgcagg

SEQ ID NO: 34 - DNA, 13 bp, Artificial Sequence
  Synthetic oligonucleotide
gcccggcttgcag

SEQ ID NO: 35 - DNA, 13 bp, Artificial Sequence
  Synthetic oligonucleotide
agcccggcttgca

SEQ ID NO: 36 - DNA, 13 bp, Artificial Sequence
  Synthetic oligonucleotide
tagcccggcttgc

SEQ ID NO: 37 - DNA, 13 bp, Artificial Sequence
  Synthetic oligonucleotide
atagcccggcttg

SEQ ID NO: 38 - DNA, 13 bp, Artificial Sequence
  Synthetic oligonucleotide
catagcccggctt

SEQ ID NO: 39 - DNA, 13 bp, Artificial Sequence
  Synthetic oligonucleotide
acacatagcccgg

SEQ ID NO: 40 - DNA, 13 bp, Artificial Sequence
  Synthetic oligonucleotide
gacacatagcccg

SEQ ID NO: 41 - DNA, 13 bp, Artificial Sequence
  Synthetic oligonucleotide
tgtgagagggcag

SEQ ID NO: 42 - DNA, 13 bp, Artificial Sequence
  Synthetic oligonucleotide
ttgatgggccaca

SEQ ID NO: 43 - DNA, 13 bp, Artificial Sequence
  Synthetic oligonucleotide
gttgatgggccac

SEQ ID NO: 44 - DNA, 13 bp, Artificial Sequence
  Synthetic oligonucleotide
tgttgatgggcca

SEQ ID NO: 45 - DNA, 13 bp, Artificial Sequence
  Synthetic oligonucleotide
gtgttgatgggcc

SEQ ID NO: 46 - DNA, 13 bp, Artificial Sequence
  Synthetic oligonucleotide
ggacatactctgg

SEQ ID NO: 47 - DNA, 13 bp, Artificial Sequence
  Synthetic oligonucleotide
aggacatactctg

SEQ ID NO: 48 - DNA, 13 bp, Artificial Sequence
  Synthetic oligonucleotide
aaggacatactct

SEQ ID NO: 49 - DNA, 13 bp, Artificial Sequence
  Synthetic oligonucleotide
aaaggacatactc

SEQ ID NO: 50 - DNA, 13 bp, Artificial Sequence
  Synthetic oligonucleotide
gctccattttcta

SEQ ID NO: 51 - DNA, 13 bp, Artificial Sequence
  Synthetic oligonucleotide
gggatattcaaaa

SEQ ID NO: 52 - DNA, 13 bp, Artificial Sequence
  Synthetic oligonucleotide
tgggatattcaaa

SEQ ID NO: 53 - DNA, 13 bp, Artificial Sequence
  Synthetic oligonucleotide
ttgggatattcaa

SEQ ID NO: 54 - DNA, 13 bp, Artificial Sequence
  Synthetic oligonucleotide
gttgggatattca

SEQ ID NO: 55 - DNA, 13 bp, Artificial Sequence
  Synthetic oligonucleotide
gcacttggcagaa

SEQ ID NO: 56 - DNA, 13 bp, Artificial Sequence
  Synthetic oligonucleotide
tgcacttggcaga

SEQ ID NO: 57 - DNA, 13 bp, Artificial Sequence
  Synthetic oligonucleotide
gtgcacttggcag

SEQ ID NO: 58 - DNA, 13 bp, Artificial Sequence
  Synthetic oligonucleotide
agtgcacttggca

SEQ ID NO: 59 - DNA, 13 bp, Artificial Sequence
  Synthetic oligonucleotide
cagtgcacttggc

SEQ ID NO: 60 - DNA, 13 bp, Artificial Sequence
  Synthetic oligonucleotide
ctcagtgcacttg

SEQ ID NO: 61 - DNA, 13 bp, Artificial Sequence
  Synthetic oligonucleotide
cctcagtgcactt

SEQ ID NO: 62 - DNA, 13 bp, Artificial Sequence
  Synthetic oligonucleotide
tcctcagtgcact

SEQ ID NO: 63 - DNA, 13 bp, Artificial Sequence
  Synthetic oligonucleotide
ttcctcagtgcac

SEQ ID NO: 64 - DNA, 13 bp, Artificial Sequence
  Synthetic oligonucleotide
cttcctcagtgca

SEQ ID NO: 65 - DNA, 13 bp, Artificial Sequence
  Synthetic oligonucleotide
ccttcctcagtgc

SEQ ID NO: 66 - DNA, 13 bp, Artificial Sequence
  Synthetic oligonucleotide
tccttcctcagtg

SEQ ID NO: 67 - DNA, 13 bp, Artificial Sequence
  Synthetic oligonucleotide
gctgatggcttat

SEQ ID NO: 68 - DNA, 13 bp, Artificial Sequence
  Synthetic oligonucleotide
agctgatggctta

SEQ ID NO: 69 - DNA, 13 bp, Artificial Sequence
  Synthetic oligonucleotide
cagctgatggctt

SEQ ID NO: 70 - DNA, 13 bp, Artificial Sequence
  Synthetic oligonucleotide
ccagctgatggct

SEQ ID NO: 71 - DNA, 13 bp, Artificial Sequence
  Synthetic oligonucleotide
tccagctgatggc

SEQ ID NO: 72 - DNA, 13 bp, Artificial Sequence
  Synthetic oligonucleotide
ttccagctgatgg

SEQ ID NO: 73 - DNA, 13 bp, Artificial Sequence
  Synthetic oligonucleotide
gtttccagctgat

SEQ ID NO: 74 - DNA, 13 bp, Artificial Sequence
  Synthetic oligonucleotide
tgtttccagctga

SEQ ID NO: 75 - DNA, 13 bp, Artificial Sequence
  Synthetic oligonucleotide
ttgtttccagctg

SEQ ID NO: 76 - DNA, 13 bp, Artificial Sequence
  Synthetic oligonucleotide
aactgctgtgtcc

SEQ ID NO: 77 - DNA, 13 bp, Artificial Sequence
  Synthetic oligonucleotide
aaaaactgctgtg

SEQ ID NO: 78 - DNA, 13 bp, Artificial Sequence
  Synthetic oligonucleotide
tggcaaacattca

SEQ ID NO: 79 - DNA, 13 bp, Artificial Sequence
  Synthetic oligonucleotide
gtggcaaacattc

SEQ ID NO: 80 - DNA, 13 bp, Artificial Sequence
  Synthetic oligonucleotide
gttgtggcaaaca

SEQ ID NO: 81 - DNA, 13 bp, Artificial Sequence
  Synthetic oligonucleotide
tgttgtggcaaac

SEQ ID NO: 82 - DNA, 13 bp, Artificial Sequence
  Synthetic oligonucleotide
gcatgttgtggca

SEQ ID NO: 83 - DNA, 13 bp, Artificial Sequence
  Synthetic oligonucleotide
cgcatgttgtggc

SEQ ID NO: 84 - DNA, 13 bp, Artificial Sequence
  Synthetic oligonucleotide
tcgcatgttgtgg

SEQ ID NO: 85 - DNA, 13 bp, Artificial Sequence
  Synthetic oligonucleotide
atcgcatgttgtg

SEQ ID NO: 86 - DNA, 13 bp, Artificial Sequence
  Synthetic oligonucleotide
acatcgcatgttg

SEQ ID NO: 87 - DNA, 13 bp, Artificial Sequence
  Synthetic oligonucleotide
ttccaaacatcgc

SEQ ID NO: 88 - DNA, 13 bp, Artificial Sequence
  Synthetic oligonucleotide
agtccaatttcca

SEQ ID NO: 89 - DNA, 13 bp, Artificial Sequence
  Synthetic oligonucleotide
ccctgcattctgg

SEQ ID NO: 90 - DNA, 13 bp, Artificial Sequence
  Synthetic oligonucleotide
tccctgcattctg

SEQ ID NO: 91 - DNA, 13 bp, Artificial Sequence
  Synthetic oligonucleotide
ttccctgcattct

SEQ ID NO: 92 - DNA, 13 bp, Artificial Sequence
  Synthetic oligonucleotide
attgtctggtctt

SEQ ID NO: 93 - DNA, 13 bp, Artificial Sequence
  Synthetic oligonucleotide
cattgtctggtct

SEQ ID NO: 94 - DNA, 13 bp, Artificial Sequence
  Synthetic oligonucleotide
ccattgtctggtc

SEQ ID NO: 95 - DNA, 13 bp, Artificial Sequence
  Synthetic oligonucleotide
tccattgtctggt

SEQ ID NO: 96 - DNA, 13 bp, Artificial Sequence
  Synthetic oligonucleotide
atccattgtctgg

SEQ ID NO: 97 - DNA, 13 bp, Artificial Sequence
  Synthetic oligonucleotide
aatccattgtctg

SEQ ID NO: 98 - DNA, 13 bp, Artificial Sequence
  Synthetic oligonucleotide
gcaggatagttca

SEQ ID NO: 99 - DNA, 13 bp, Artificial Sequence
  Synthetic oligonucleotide
ggttttgcaggat

SEQ ID NO: 100 - DNA, 13 bp, Artificial Sequence
  Synthetic oligonucleotide
tggttttgcagga

SEQ ID NO: 101 - DNA, 13 bp, Artificial Sequence
  Synthetic oligonucleotide
ggctttatccttg

SEQ ID NO: 102 - DNA, 13 bp, Artificial Sequence
  Synthetic oligonucleotide
gtggctttatcct

SEQ ID NO: 103 - DNA, 13 bp, Artificial Sequence
  Synthetic oligonucleotide
atgtggctttatc

SEQ ID NO: 104 - DNA, 13 bp, Artificial Sequence
  Synthetic oligonucleotide
aatgtggctttat

SEQ ID NO: 105 - DNA, 13 bp, Artificial Sequence
  Synthetic oligonucleotide
ccaaatgtggctt

SEQ ID NO: 106 - DNA, 13 bp, Artificial Sequence
  Synthetic oligonucleotide
catcatggcagcc

SEQ ID NO: 107 - DNA, 13 bp, Artificial Sequence
  Synthetic oligonucleotide
ccatcatggcagc

SEQ ID NO: 108 - DNA, 13 bp, Artificial Sequence
  Synthetic oligonucleotide
tccatcatggcag

SEQ ID NO: 109 - DNA, 13 bp, Artificial Sequence
  Synthetic oligonucleotide
atccatcatggca

SEQ ID NO: 110 - DNA, 13 bp, Artificial Sequence
  Synthetic oligonucleotide
tatccatcatggc

SEQ ID NO: 111 - DNA, 13 bp, Artificial Sequence
  Synthetic oligonucleotide
atatccatcatgg

SEQ ID NO: 112 - DNA, 13 bp, Artificial Sequence
  Synthetic oligonucleotide
cccagtttggtac

SEQ ID NO: 113 - DNA, 13 bp, Artificial Sequence
  Synthetic oligonucleotide
tcccagtttggta

SEQ ID NO: 114 - DNA, 13 bp, Artificial Sequence
  Synthetic oligonucleotide
ttcccagtttggt

SEQ ID NO: 115 - DNA, 13 bp, Artificial Sequence
  Synthetic oligonucleotide
tttcccagtttgg

SEQ ID NO: 116 - DNA, 13 bp, Artificial Sequence
  Synthetic oligonucleotide
gtttcccagtttg

SEQ ID NO: 117 - DNA, 13 bp, Artificial Sequence
  Synthetic oligonucleotide
cagtttcccagtt

SEQ ID NO: 118 - DNA, 13 bp, Artificial Sequence
  Synthetic oligonucleotide
ccagtttcccagt

SEQ ID NO: 119 - DNA, 13 bp, Artificial Sequence
  Synthetic oligonucleotide
accagtttcccag

SEQ ID NO: 120 - DNA, 13 bp, Artificial Sequence
  Synthetic oligonucleotide
gaccagtttccca

SEQ ID NO: 121 - DNA, 13 bp, Artificial Sequence
  Synthetic oligonucleotide
agaccagtttccc

SEQ ID NO: 122 - DNA, 13 bp, Artificial Sequence
  Synthetic oligonucleotide
gcagaccagtttc

SEQ ID NO: 123 - DNA, 13 bp, Artificial Sequence
  Synthetic oligonucleotide
ggcagaccagttt

SEQ ID NO: 124 - DNA, 13 bp, Artificial Sequence
  Synthetic oligonucleotide
tggcagaccagtt

SEQ ID NO: 125 - DNA, 13 bp, Artificial Sequence
  Synthetic oligonucleotide
atggcagaccagt

SEQ ID NO: 126 - DNA, 13 bp, Artificial Sequence
  Synthetic oligonucleotide
catggcagaccag

SEQ ID NO: 127 - DNA, 13 bp, Artificial Sequence
  Synthetic oligonucleotide
gcatggcagacca

SEQ ID NO: 128 - DNA, 13 bp, Artificial Sequence
  Synthetic oligonucleotide
ggcatggcagacc

SEQ ID NO: 129 - DNA, 13 bp, Artificial Sequence
  Synthetic oligonucleotide
tggcatggcagac

SEQ ID NO: 130 - DNA, 13 bp, Artificial Sequence
  Synthetic oligonucleotide
ttggcatggcaga

SEQ ID NO: 131 - DNA, 13 bp, Artificial Sequence
  Synthetic oligonucleotide
cttggcatggcag

SEQ ID NO: 132 - DNA, 13 bp, Artificial Sequence
  Synthetic oligonucleotide
acttggcatggca

SEQ ID NO: 133 - DNA, 13 bp, Artificial Sequence
  Synthetic oligonucleotide
aacttggcatggc

SEQ ID NO: 134 - DNA, 13 bp, Artificial Sequence
  Synthetic oligonucleotide
caacttggcatgg

SEQ ID NO: 135 - DNA, 13 bp, Artificial Sequence
  Synthetic oligonucleotide
acaacttggcatg

SEQ ID NO: 136 - DNA, 13 bp, Artificial Sequence
  Synthetic oligonucleotide
gctttacaacttg

SEQ ID NO: 137 - DNA, 13 bp, Artificial Sequence
  Synthetic oligonucleotide
tcacaggtacttt

SEQ ID NO: 138 - DNA, 13 bp, Artificial Sequence
  Synthetic oligonucleotide
acaccacagtggc

SEQ ID NO: 139 - DNA, 13 bp, Artificial Sequence
  Synthetic oligonucleotide
gtacaccacagtg

SEQ ID NO: 140 - DNA, 13 bp, Artificial Sequence
  Synthetic oligonucleotide
ggtacaccacagt

SEQ ID NO: 141 - DNA, 13 bp, Artificial Sequence
  Synthetic oligonucleotide
tggtacaccacag

SEQ ID NO: 142 - DNA, 13 bp, Artificial Sequence
  Synthetic oligonucleotide
ttggtacaccaca

SEQ ID NO: 143 - DNA, 13 bp, Artificial Sequence
  Synthetic oligonucleotide
cttggtacaccac

SEQ ID NO: 144 - DNA, 13 bp, Artificial Sequence
  Synthetic oligonucleotide
ccttggtacacca

SEQ ID NO: 145 - DNA, 13 bp, Artificial Sequence
  Synthetic oligonucleotide
tccttggtacacc

SEQ ID NO: 146 - DNA, 13 bp, Artificial Sequence
  Synthetic oligonucleotide
ctccttggtacac

SEQ ID NO: 147 - DNA, 13 bp, Artificial Sequence
  Synthetic oligonucleotide
tctccttggtaca

SEQ ID NO: 148 - DNA, 13 bp, Artificial Sequence
  Synthetic oligonucleotide
ctctccttggtac

SEQ ID NO: 149 - DNA, 13 bp, Artificial Sequence
  Synthetic oligonucleotide
tctctccttggta

SEQ ID NO: 150 - DNA, 13 bp, Artificial Sequence
  Synthetic oligonucleotide
ctctctccttggt

SEQ ID NO: 151 - DNA, 13 bp, Artificial Sequence
  Synthetic oligonucleotide
tctctctccttgg

SEQ ID NO: 152 - DNA, 13 bp, Artificial Sequence
  Synthetic oligonucleotide
ctctctctccttg

SEQ ID NO: 153 - DNA, 13 bp, Artificial Sequence
  Synthetic oligonucleotide
actctctctcctt

SEQ ID NO: 154 - DNA, 13 bp, Artificial Sequence
  Synthetic oligonucleotide
tactctctctcct

SEQ ID NO: 155 - DNA, 13 bp, Artificial Sequence
  Synthetic oligonucleotide
ttactctctctcc

SEQ ID NO: 156 - DNA, 13 bp, Artificial Sequence
  Synthetic oligonucleotide
gcattccattctt

SEQ ID NO: 157 - DNA, 13 bp, Artificial Sequence
  Synthetic oligonucleotide
agcattccattct

SEQ ID NO: 158 - DNA, 13 bp, Artificial Sequence
  Synthetic oligonucleotide
agctacacttctt

SEQ ID NO: 159 - DNA, 13 bp, Artificial Sequence
  Synthetic oligonucleotide
tagctacacttct

SEQ ID NO: 160 - DNA, 13 bp, Artificial Sequence
  Synthetic oligonucleotide
ttgaagcatttgg

SEQ ID NO: 161 - DNA, 13 bp, Artificial Sequence
  Synthetic oligonucleotide
gttccttgaagca

SEQ ID NO: 162 - DNA, 13 bp, Artificial Sequence
  Synthetic oligonucleotide
tgttccttgaagc

SEQ ID NO: 163 - DNA, 13 bp, Artificial Sequence
  Synthetic oligonucleotide
gtgttccttgaag

SEQ ID NO: 164 - DNA, 13 bp, Artificial Sequence
  Synthetic oligonucleotide
tgtgttccttgaa

SEQ ID NO: 165 - DNA, 13 bp, Artificial Sequence
  Synthetic oligonucleotide
ctgtgttccttga

SEQ ID NO: 166 - DNA, 13 bp, Artificial Sequence
  Synthetic oligonucleotide
actgtgttccttg

SEQ ID NO: 167 - DNA, 13 bp, Artificial Sequence
  Synthetic oligonucleotide
aactgtgttcctt

SEQ ID NO: 168 - DNA, 13 bp, Artificial Sequence
  Synthetic oligonucleotide
gaactgtgttcct

SEQ ID NO: 169 - DNA, 13 bp, Artificial Sequence
  Synthetic oligonucleotide
agaactgtgttcc

SEQ ID NO: 170 - DNA, 13 bp, Artificial Sequence
  Synthetic oligonucleotide
gagaactgtgttc

SEQ ID NO: 171 - DNA, 13 bp, Artificial Sequence
  Synthetic oligonucleotide
agagaactgtgtt

SEQ ID NO: 172 - DNA, 13 bp, Artificial Sequence
  Synthetic oligonucleotide
cagagaactgtgt

SEQ ID NO: 173 - DNA, 13 bp, Artificial Sequence
  Synthetic oligonucleotide
ccagagaactgtg

SEQ ID NO: 174 - DNA, 13 bp, Artificial Sequence
  Synthetic oligonucleotide
gccagagaactgt

SEQ ID NO: 175 - DNA, 13 bp, Artificial Sequence
  Synthetic oligonucleotide
agccagagaactg

SEQ ID NO: 176 - DNA, 13 bp, Artificial Sequence
  Synthetic oligonucleotide
aagccagagaact

SEQ ID NO: 177 - DNA, 13 bp, Artificial Sequence
  Synthetic oligonucleotide
gcatcagttttcc

SEQ ID NO: 178 - DNA, 13 bp, Artificial Sequence
  Synthetic oligonucleotide
tgcatcagttttc

SEQ ID NO: 179 - DNA, 13 bp, Artificial Sequence
  Synthetic oligonucleotide
gatgcatcagttt

SEQ ID NO: 180 - DNA, 13 bp, Artificial Sequence
  Synthetic oligonucleotide
ggatgcatcagtt

SEQ ID NO: 181 - DNA, 13 bp, Artificial Sequence
  Synthetic oligonucleotide
cggatgcatcagt

SEQ ID NO: 182 - DNA, 13 bp, Artificial Sequence
  Synthetic oligonucleotide
tcggatgcatcag

SEQ ID NO: 183 - DNA, 13 bp, Artificial Sequence
  Synthetic oligonucleotide
atcggatgcatca

SEQ ID NO: 184 - DNA, 13 bp, Artificial Sequence
  Synthetic oligonucleotide
catcggatgcatc

SEQ ID NO: 185 - DNA, 13 bp, Artificial Sequence
  Synthetic oligonucleotide
acatcggatgcat

SEQ ID NO: 186 - DNA, 13 bp, Artificial Sequence
  Synthetic oligonucleotide
tacatcggatgca

SEQ ID NO: 187 - DNA, 13 bp, Artificial Sequence
  Synthetic oligonucleotide
ttacatcggatgc

SEQ ID NO: 188 - DNA, 13 bp, Artificial Sequence
  Synthetic oligonucleotide
gctttacatcgga

SEQ ID NO: 189 - DNA, 13 bp, Artificial Sequence
  Synthetic oligonucleotide
ggctttacatcgg

SEQ ID NO: 190 - DNA, 13 bp, Artificial Sequence
  Synthetic oligonucleotide
tggctttacatcg

SEQ ID NO: 191 - DNA, 13 bp, Artificial Sequence
  Synthetic oligonucleotide
tgtggaatctgaa

SEQ ID NO: 192 - DNA, 13 bp, Artificial Sequence
  Synthetic oligonucleotide
agtgtgacatttt

SEQ ID NO: 193 - DNA, 13 bp, Artificial Sequence
  Synthetic oligonucleotide
aagtgtgacattt

SEQ ID NO: 194 - DNA, 13 bp, Artificial Sequence
  Synthetic oligonucleotide
ccttggatgaaca

SEQ ID NO: 195 - DNA, 13 bp, Artificial Sequence
  TCCTTGGATGAAC
tccttggatgaac

SEQ ID NO: 196 - DNA, 13 bp, Artificial Sequence
  Synthetic oligonucleotide
gttccttggatga

SEQ ID NO: 197 - DNA, 13 bp, Artificial Sequence
  Synthetic oligonucleotide
ggttccttggatg

SEQ ID NO: 198 - DNA, 13 bp, Artificial Sequence
  Synthetic oligonucleotide
aggttccttggat

SEQ ID NO: 199 - DNA, 13 bp, Artificial Sequence
  Synthetic oligonucleotide
taggttccttgga

SEQ ID NO: 200 - DNA, 13 bp, Artificial Sequence
  Synthetic oligonucleotide
ttaggttccttgg

SEQ ID NO: 201 - DNA, 13 bp, Homo sapiens
ttctccagtgctc

SEQ ID NO: 202 - DNA, 13 bp, Homo sapiens
tctccagtgctca

SEQ ID NO: 203 - DNA, 13 bp, Homo sapiens
ccagtgctcatct

SEQ ID NO: 204 - DNA, 13 bp, Homo sapiens
ctgccatgttgct

SEQ ID NO: 205 - DNA, 13 bp, Homo sapiens
tgccatgttgcta

SEQ ID NO: 206 - DNA, 13 bp, Homo sapiens
gccatgttgctat

SEQ ID NO: 207 - DNA, 13 bp, Homo sapiens
catgttgctattg

SEQ ID NO: 208 - DNA, 13 bp, Homo sapiens
atgttgctattgc

SEQ ID NO: 209 - DNA, 13 bp, Homo sapiens
ttgctattgcagg

SEQ ID NO: 210 - DNA, 13 bp, Homo sapiens
tgctattgcagga

SEQ ID NO: 211 - DNA, 13 bp, Homo sapiens
gctattgcaggac

SEQ ID NO: 212 - DNA, 13 bp, Homo sapiens
attgcaggacgga

SEQ ID NO: 213 - DNA, 13 bp, Homo sapiens
ttgcaggacggac

SEQ ID NO: 214 - DNA, 13 bp, Homo sapiens
tgcaggacggacc

SEQ ID NO: 215 - DNA, 13 bp, Homo sapiens
gcaggacggacct

SEQ ID NO: 216 - DNA, 13 bp, Homo sapiens
caggacggacctg

SEQ ID NO: 217 - DNA, 13 bp, Homo sapiens
cggacctgtccca

SEQ ID NO: 218 - DNA, 13 bp, Homo sapiens
ggacctgtcccaa

SEQ ID NO: 219 - DNA, 13 bp, Homo sapiens
cctgtcccaagcc

SEQ ID NO: 220 - DNA, 13 bp, Homo sapiens
ctgtcccaagcca

SEQ ID NO: 221 - DNA, 13 bp, Homo sapiens
tgtcccaagccag

SEQ ID NO: 222 - DNA, 13 bp, Homo sapiens
gtcccaagccaga

SEQ ID NO: 223 - DNA, 13 bp, Homo sapiens
cccaagccagatg

SEQ ID NO: 224 - DNA, 13 bp, Homo sapiens
ccaagccagatga

SEQ ID NO: 225 - DNA, 13 bp, Homo sapiens
caagccagatgat

SEQ ID NO: 226 - DNA, 13 bp, Homo sapiens
aagccagatgatt

SEQ ID NO: 227 - DNA, 13 bp, Homo sapiens
agccagatgattt

SEQ ID NO: 228 - DNA, 13 bp, Homo sapiens
ctatgagccagga

SEQ ID NO: 229 - DNA, 13 bp, Homo sapiens
tatgagccaggag

SEQ ID NO: 230 - DNA, 13 bp, Homo sapiens
atgagccaggaga

SEQ ID NO: 231 - DNA, 13 bp, Homo sapiens
tcctgcaagccgg

SEQ ID NO: 232 - DNA, 13 bp, Homo sapiens
cctgcaagccggg

SEQ ID NO: 233 - DNA, 13 bp, Homo sapiens
ctgcaagccgggc

SEQ ID NO: 234 - DNA, 13 bp, Homo sapiens
tgcaagccgggct

SEQ ID NO: 235 - DNA, 13 bp, Homo sapiens
gcaagccgggcta

SEQ ID NO: 236 - DNA, 13 bp, Homo sapiens
caagccgggctat

SEQ ID NO: 237 - DNA, 13 bp, Homo sapiens
aagccgggctatg

SEQ ID NO: 238 - DNA, 13 bp, Homo sapiens
ccgggctatgtgt

SEQ ID NO: 239 - DNA, 13 bp, Homo sapiens
cgggctatgtgtc

SEQ ID NO: 240 - DNA, 13 bp, Homo sapiens
ctgccctctcaca

SEQ ID NO: 241 - DNA, 13 bp, Homo sapiens
tgtggcccatcaa

SEQ ID NO: 242 - DNA, 13 bp, Homo sapiens
gtggcccatcaac

SEQ ID NO: 243 - DNA, 13 bp, Homo sapiens
tggcccatcaaca

SEQ ID NO: 244 - DNA, 13 bp, Homo sapiens
ggcccatcaacac

SEQ ID NO: 245 - DNA, 13 bp, Homo sapiens
ccagagtatgtcc

SEQ ID NO: 246 - DNA, 13 bp, Homo sapiens
cagagtatgtcct

SEQ ID NO: 247 - DNA, 13 bp, Homo sapiens
agagtatgtcctt

SEQ ID NO: 248 - DNA, 13 bp, Homo sapiens
gagtatgtccttt

SEQ ID NO: 249 - DNA, 13 bp, Homo sapiens
tagaaaatggagc

SEQ ID NO: 250 - DNA, 13 bp, Homo sapiens
ttttgaatatccc

SEQ ID NO: 251 - DNA, 13 bp, Homo sapiens
tttgaatatccca

SEQ ID NO: 252 - DNA, 13 bp, Homo sapiens
ttgaatatcccaa

SEQ ID NO: 253 - DNA, 13 bp, Homo sapiens
tgaatatcccaac

SEQ ID NO: 254 - DNA, 13 bp, Homo sapiens
ttctgccaagtgc

SEQ ID NO: 255 - DNA, 13 bp, Homo sapiens
tctgccaagtgca

SEQ ID NO: 256 - DNA, 13 bp, Homo sapiens
ctgccaagtgcac

SEQ ID NO: 257 - DNA, 13 bp, Homo sapiens
tgccaagtgcact

SEQ ID NO: 258 - DNA, 13 bp, Homo sapiens
gccaagtgcactg

SEQ ID NO: 259 - DNA, 13 bp, Homo sapiens
caagtgcactgag

SEQ ID NO: 260 - DNA, 13 bp, Homo sapiens
aagtgcactgagg

SEQ ID NO: 261 - DNA, 13 bp, Homo sapiens
agtgcactgagga

SEQ ID NO: 262 - DNA, 13 bp, Homo sapiens
gtgcactgaggaa

SEQ ID NO: 263 - DNA, 13 bp, Homo sapiens
tgcactgaggaag

SEQ ID NO: 264 - DNA, 13 bp, Homo sapiens
gcactgaggaagg

SEQ ID NO: 265 - DNA, 13 bp, Homo sapiens
cactgaggaagga

SEQ ID NO: 266 - DNA, 13 bp, Homo sapiens
ataagccatcagc

SEQ ID NO: 267 - DNA, 13 bp, Homo sapiens
taagccatcagct

SEQ ID NO: 268 - DNA, 13 bp, Homo sapiens
aagccatcagctg

SEQ ID NO: 269 - DNA, 13 bp, Homo sapiens
agccatcagctgg

SEQ ID NO: 270 - DNA, 13 bp, Homo sapiens
gccatcagctgga

SEQ ID NO: 271 - DNA, 13 bp, Homo sapiens
ccatcagctggaa

SEQ ID NO: 272 - DNA, 13 bp, Homo sapiens
atcagctggaaac

SEQ ID NO: 273 - DNA, 13 bp, Homo sapiens
tcagctggaaaca

SEQ ID NO: 274 - DNA, 13 bp, Homo sapiens
cagctggaaacaa

SEQ ID NO: 275 - DNA, 13 bp, Homo sapiens
ggacacagcagtt

SEQ ID NO: 276 - DNA, 13 bp, Homo sapiens
cacagcagttttt

SEQ ID NO: 277 - DNA, 13 bp, Homo sapiens
tgaatgtttgcca

SEQ ID NO: 278 - DNA, 13 bp, Homo sapiens
gaatgtttgccac

SEQ ID NO: 279 - DNA, 13 bp, Homo sapiens
tgtttgccacaac

SEQ ID NO: 280 - DNA, 13 bp, Homo sapiens
gtttgccacaaca

SEQ ID NO: 281 - DNA, 13 bp, Homo sapiens
tgccacaacatgc

SEQ ID NO: 282 - DNA, 13 bp, Homo sapiens
gccacaacatgcg

SEQ ID NO: 283 - DNA, 13 bp, Homo sapiens
ccacaacatgcga

SEQ ID NO: 284 - DNA, 13 bp, Homo sapiens
cacaacatgcgat

SEQ ID NO: 285 - DNA, 13 bp, Homo sapiens
caacatgcgatgt

SEQ ID NO: 286 - DNA, 13 bp, Homo sapiens
gcgatgtttggaa

SEQ ID NO: 287 - DNA, 13 bp, Homo sapiens
tggaaattggact

SEQ ID NO: 288 - DNA, 13 bp, Homo sapiens
ccagaatgcaggg

SEQ ID NO: 289 - DNA, 13 bp, Homo sapiens
cagaatgcaggga

SEQ ID NO: 290 - DNA, 13 bp, Homo sapiens
agaatgcagggaa

SEQ ID NO: 291 - DNA, 13 bp, Homo sapiens
aagaccagacaat

SEQ ID NO: 292 - DNA, 13 bp, Homo sapiens
agaccagacaatg

SEQ ID NO: 293 - DNA, 13 bp, Homo sapiens
gaccagacaatgg

SEQ ID NO: 294 - DNA, 13 bp, Homo sapiens
accagacaatgga

SEQ ID NO: 295 - DNA, 13 bp, Homo sapiens
ccagacaatggat

SEQ ID NO: 296 - DNA, 13 bp, Homo sapiens
cagacaatggatt

SEQ ID NO: 297 - DNA, 13 bp, Homo sapiens
tgaactatcctgc

SEQ ID NO: 298 - DNA, 13 bp, Homo sapiens
atcctgcaaaacc

SEQ ID NO: 299 - DNA, 13 bp, Homo sapiens
tcctgcaaaacca

SEQ ID NO: 300 - DNA, 13 bp, Homo sapiens
caaggataaagcc

SEQ ID NO: 301 - DNA, 13 bp, Homo sapiens
aggataaagccac

SEQ ID NO: 302 - DNA, 13 bp, Homo sapiens
gataaagccacat

SEQ ID NO: 303 - DNA, 13 bp, Homo sapiens
ataaagccacatt

SEQ ID NO: 304 - DNA, 13 bp, Homo sapiens
aagccacatttgg

SEQ ID NO: 305 - DNA, 13 bp, Homo sapiens
ggctgccatgatg

SEQ ID NO: 306 - DNA, 13 bp, Homo sapiens
gctgccatgatgg

SEQ ID NO: 307 - DNA, 13 bp, Homo sapiens
ctgccatgatgga

SEQ ID NO: 308 - DNA, 13 bp, Homo sapiens
tgccatgatggat

SEQ ID NO: 309 - DNA, 13 bp, Homo sapiens
gccatgatggata

SEQ ID NO: 310 - DNA, 13 bp, Homo sapiens
ccatgatggatat

SEQ ID NO: 311 - DNA, 13 bp, Homo sapiens
gtaccaaactggg

SEQ ID NO: 312 - DNA, 13 bp, Homo sapiens
taccaaactggga

SEQ ID NO: 313 - DNA, 13 bp, Homo sapiens
accaaactgggaa

SEQ ID NO: 314 - DNA, 13 bp, Homo sapiens
ccaaactgggaaa

SEQ ID NO: 315 - DNA, 13 bp, Homo sapiens
caaactgggaaac

SEQ ID NO: 316 - DNA, 13 bp, Homo sapiens
aactgggaaactg

SEQ ID NO: 317 - DNA, 13 bp, Homo sapiens
actgggaaactgg

SEQ ID NO: 318 - DNA, 13 bp, Homo sapiens
ctgggaaactggt

SEQ ID NO: 319 - DNA, 13 bp, Homo sapiens
tgggaaactggtc

SEQ ID NO: 320 - DNA, 13 bp, Homo sapiens
gggaaactggtct

SEQ ID NO: 321 - DNA, 13 bp, Homo sapiens
gaaactggtctgc

SEQ ID NO: 322 - DNA, 13 bp, Homo sapiens
aaactggtctgcc

SEQ ID NO: 323 - DNA, 13 bp, Homo sapiens
aactggtctgcca

SEQ ID NO: 324 - DNA, 13 bp, Homo sapiens
actggtctgccat

SEQ ID NO: 325 - DNA, 13 bp, Homo sapiens
ctggtctgccatg

SEQ ID NO: 326 - DNA, 13 bp, Homo sapiens
tggtctgccatgc

SEQ ID NO: 327 - DNA, 13 bp, Homo sapiens
ggtctgccatgcc

SEQ ID NO: 328 - DNA, 13 bp, Homo sapiens
gtctgccatgcca

SEQ ID NO: 329 - DNA, 13 bp, Homo sapiens
tctgccatgccaa

SEQ ID NO: 330 - DNA, 13 bp, Homo sapiens
ctgccatgccaag

SEQ ID NO: 331 - DNA, 13 bp, Homo sapiens
tgccatgccaagt

SEQ ID NO: 332 - DNA, 13 bp, Homo sapiens
gccatgccaagtt

SEQ ID NO: 333 - DNA, 13 bp, Homo sapiens
ccatgccaagttg

SEQ ID NO: 334 - DNA, 13 bp, Homo sapiens
catgccaagttgt

SEQ ID NO: 335 - DNA, 13 bp, Homo sapiens
caagttgtaaagc

SEQ ID NO: 336 - DNA, 13 bp, Homo sapiens
aaagtacctgtga

SEQ ID NO: 337 - DNA, 13 bp, Homo sapiens
gccactgtggtgt

SEQ ID NO: 338 - DNA, 13 bp, Homo sapiens
cactgtggtgtac

SEQ ID NO: 339 - DNA, 13 bp, Homo sapiens
actgtggtgtacc

SEQ ID NO: 340 - DNA, 13 bp, Homo sapiens
ctgtggtgtacca

SEQ ID NO: 341 - DNA, 13 bp, Homo sapiens
tgtggtgtaccaa

SEQ ID NO: 342 - DNA, 13 bp, Homo sapiens
gtggtgtaccaag

SEQ ID NO: 343 - DNA, 13 bp, Homo sapiens
tggtgtaccaagg

SEQ ID NO: 344 - DNA, 13 bp, Homo sapiens
ggtgtaccaagga

SEQ ID NO: 345 - DNA, 13 bp, Homo sapiens
gtgtaccaaggag

SEQ ID NO: 346 - DNA, 13 bp, Homo sapiens
tgtaccaaggaga

SEQ ID NO: 347 - DNA, 13 bp, Homo sapiens
gtaccaaggagag

SEQ ID NO: 348 - DNA, 13 bp, Homo sapiens
taccaaggagaga

SEQ ID NO: 349 - DNA, 13 bp, Homo sapiens
accaaggagagag

SEQ ID NO: 350 - DNA, 13 bp, Homo sapiens
ccaaggagagaga

SEQ ID NO: 351 - DNA, 13 bp, Homo sapiens
caaggagagagag

SEQ ID NO: 352 - DNA, 13 bp, Homo sapiens
aaggagagagagt

SEQ ID NO: 353 - DNA, 13 bp, Homo sapiens
aggagagagagta

SEQ ID NO: 354 - DNA, 13 bp, Homo sapiens
ggagagagagtaa

SEQ ID NO: 355 - DNA, 13 bp, Homo sapiens
aagaatggaatgc

SEQ ID NO: 356 - DNA, 13 bp, Homo sapiens
agaatggaatgct

SEQ ID NO: 357 - DNA, 13 bp, Homo sapiens
aagaagtgtagct

SEQ ID NO: 358 - DNA, 13 bp, Homo sapiens
agaagtgtagcta

SEQ ID NO: 359 - DNA, 13 bp, Homo sapiens
ccaaatgcttcaa

SEQ ID NO: 360 - DNA, 13 bp, Homo sapiens
tgcttcaaggaac

SEQ ID NO: 361 - DNA, 13 bp, Homo sapiens
gcttcaaggaaca

SEQ ID NO: 362 - DNA, 13 bp, Homo sapiens
cttcaaggaacac

SEQ ID NO: 363 - DNA, 13 bp, Homo sapiens
ttcaaggaacaca

SEQ ID NO: 364 - DNA, 13 bp, Homo sapiens
tcaaggaacacag

SEQ ID NO: 365 - DNA, 13 bp, Homo sapiens
caaggaacacagt

SEQ ID NO: 366 - DNA, 13 bp, Homo sapiens
aaggaacacagtt

SEQ ID NO: 367 - DNA, 13 bp, Homo sapiens
aggaacacagttc

SEQ ID NO: 368 - DNA, 13 bp, Homo sapiens
ggaacacagttct

SEQ ID NO: 369 - DNA, 13 bp, Homo sapiens
gaacacagttctc

SEQ ID NO: 370 - DNA, 13 bp, Homo sapiens
aacacagttctct

SEQ ID NO: 371 - DNA, 13 bp, Homo sapiens
acacagttctctg

SEQ ID NO: 372 - DNA, 13 bp, Homo sapiens
cacagttctctgg

SEQ ID NO: 373 - DNA, 13 bp, Homo sapiens
acagttctctggc

SEQ ID NO: 374 - DNA, 13 bp, Homo sapiens
cagttctctggct

SEQ ID NO: 375 - DNA, 13 bp, Homo sapiens
agttctctggctt

SEQ ID NO: 376 - DNA, 13 bp, Homo sapiens
ggaaaactgatgc

SEQ ID NO: 377 - DNA, 13 bp, Homo sapiens
gaaaactgatgca

SEQ ID NO: 378 - DNA, 13 bp, Homo sapiens
aaactgatgcatc

SEQ ID NO: 379 - DNA, 13 bp, Homo sapiens
aactgatgcatcc

SEQ ID NO: 380 - DNA, 13 bp, Homo sapiens
actgatgcatccg

SEQ ID NO: 381 - DNA, 13 bp, Homo sapiens
ctgatgcatccga

SEQ ID NO: 382 - DNA, 13 bp, Homo sapiens
tgatgcatccgat

SEQ ID NO: 383 - DNA, 13 bp, Homo sapiens
gatgcatccgatg

SEQ ID NO: 384 - DNA, 13 bp, Homo sapiens
atgcatccgatgt

SEQ ID NO: 385 - DNA, 13 bp, Homo sapiens
tgcatccgatgta

SEQ ID NO: 386 - DNA, 13 bp, Homo sapiens
gcatccgatgtaa

SEQ ID NO: 387 - DNA, 13 bp, Homo sapiens
tccgatgtaaagc

SEQ ID NO: 388 - DNA, 13 bp, Homo sapiens
ccgatgtaaagcc

SEQ ID NO: 389 - DNA, 13 bp, Homo sapiens
cgatgtaaagcca

SEQ ID NO: 390 - DNA, 13 bp, Homo sapiens
ttcagattccaca

SEQ ID NO: 391 - DNA, 13 bp, Homo sapiens
aaaatgtcacact

SEQ ID NO: 392 - DNA, 13 bp, Homo sapiens
aaatgtcacactt

SEQ ID NO: 393 - DNA, 13 bp, Homo sapiens
tgttcatccaagg

SEQ ID NO: 394 - DNA, 13 bp, Homo sapiens
gttcatccaagga

SEQ ID NO: 395 - DNA, 13 bp, Homo sapiens
tcatccaaggaac

SEQ ID NO: 396 - DNA, 13 bp, Homo sapiens
catccaaggaacc

SEQ ID NO: 397 - DNA, 13 bp, Homo sapiens
atccaaggaacct

SEQ ID NO: 398 - DNA, 13 bp, Homo sapiens
tccaaggaaccta

SEQ ID NO: 399 - DNA, 13 bp, Homo sapiens
ccaaggaacctaa

SEQ ID NO: 400 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): gm
  modified_base (2)..(2): 2′-O-methyladenosine
  modified_base (11)..(11): gm
  modified_base (12)..(12): 2′-O-methyladenosine
  modified_base (13)..(13): 2′-O-methyladenosine
gagcactggagaa

SEQ ID NO: 401 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): um
  modified_base (2)..(2): gm
  modified_base (11)..(11): 2′-O-methyladenosine
  modified_base (12)..(12): gm
  modified_base (13)..(13): 2′-O-methyladenosine
ugagcactggaga

SEQ ID NO: 402 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): 2′-O-methyladenosine
  modified_base (2)..(2): gm
  modified_base (11)..(11): um
  modified_base (12)..(12): gm
  modified_base (13)..(13): gm
agatgagcacugg

SEQ ID NO: 403 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): 2′-O-methyladenosine
  modified_base (2)..(2): gm
  modified_base (11)..(11): cm
  modified_base (12)..(12): 2′-O-methyladenosine
  modified_base (13)..(13): gm
agcaacatggcag

SEQ ID NO: 404 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): um
  modified_base (2)..(2): 2′-O-methyladenosine
  modified_base (11)..(11): gm
  modified_base (12)..(12): cm
  modified_base (13)..(13): 2′-O-methyladenosine
uagcaacatggca

SEQ ID NO: 405 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): 2′-O-methyladenosine
  modified_base (2)..(2): um
  modified_base (11)..(11): gm
  modified_base (12)..(12): gm
  modified_base (13)..(13): cm
auagcaacatggc

SEQ ID NO: 406 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): gm
  modified_base (2)..(2): um
  modified_base (11)..(11): 2′-O-methyladenosine
  modified_base (12)..(12): gm
  modified_base (13)..(13): cm
gucctgcaatagc

SEQ ID NO: 407 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): gm
  modified_base (2)..(2): um
  modified_base (11)..(11): cm
  modified_base (12)..(12): 2′-O-methyladenosine
  modified_base (13)..(13): 2′-O-methyladenosine
guccgtcctgcaa

SEQ ID NO: 408 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): gm
  modified_base (2)..(2): gm
  modified_base (11)..(11): gm
  modified_base (12)..(12): cm
  modified_base (13)..(13): 2′-O-methyladenosine
ggtccgtcctgca

SEQ ID NO: 409 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): 2′-O-methyladenosine
  modified_base (2)..(2): gm
  modified_base (11)..(11): um
  modified_base (12)..(12): gm
  modified_base (13)..(13): cm
aggtccgtccugc

SEQ ID NO: 410 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): cm
  modified_base (2)..(2): 2′-O-methyladenosine
  modified_base (11)..(11): cm
  modified_base (12)..(12): um
  modified_base (13)..(13): gm
caggtccgtccug

SEQ ID NO: 411 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): um
  modified_base (2)..(2): gm
  modified_base (11)..(11): cm
  modified_base (12)..(12): cm
  modified_base (13)..(13): gm
ugggacaggtccg

SEQ ID NO: 412 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): um
  modified_base (2)..(2): um
  modified_base (11)..(11): um
  modified_base (12)..(12): cm
  modified_base (13)..(13): cm
uugggacaggucc

SEQ ID NO: 413 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): gm
  modified_base (2)..(2): gm
  modified_base (11)..(11): 2′-O-methyladenosine
  modified_base (12)..(12): gm
  modified_base (13)..(13): gm
ggcttgggacagg

SEQ ID NO: 414 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): um
  modified_base (2)..(2): gm
  modified_base (11)..(11): cm
  modified_base (12)..(12): 2′-O-methyladenosine
  modified_base (13)..(13): gm
uggcttgggacag

SEQ ID NO: 415 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): cm
  modified_base (2)..(2): um
  modified_base (11)..(11): 2′-O-methyladenosine
  modified_base (12)..(12): cm
  modified_base (13)..(13): 2′-O-methyladenosine
cuggcttgggaca

SEQ ID NO: 416 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): um
  modified_base (2)..(2): cm
  modified_base (11)..(11): gm
  modified_base (12)..(12): 2′-O-methyladenosine
  modified_base (13)..(13): cm
uctggcttgggac

SEQ ID NO: 417 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): cm
  modified_base (2)..(2): 2′-O-methyladenosine
  modified_base (11)..(11): gm
  modified_base (12)..(12): gm
  modified_base (13)..(13): gm
catctggcttggg

SEQ ID NO: 418 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): um
  modified_base (2)..(2): cm
  modified_base (11)..(11): um
  modified_base (12)..(12): gm
  modified_base (13)..(13): gm
ucatctggctugg

SEQ ID NO: 419 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): 2′-O-methyladenosine
  modified_base (2)..(2): um
  modified_base (11)..(11): um
  modified_base (12)..(12): um
  modified_base (13)..(13): gm
aucatctggcuug

SEQ ID NO: 420 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): 2′-O-methyladenosine
  modified_base (2)..(2): 2′-O-methyladenosine
  modified_base (11)..(11): cm
  modified_base (12)..(12): um
  modified_base (13)..(13): um
aatcatctggcuu

SEQ ID NO: 421 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): 2′-O-methyladenosine
  modified_base (2)..(2): 2′-O-methyladenosine
  modified_base (11)..(11): gm
  modified_base (12)..(12): cm
  modified_base (13)..(13): um
aaatcatctggcu

SEQ ID NO: 422 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): um
  modified_base (2)..(2): cm
  modified_base (11)..(11): um
  modified_base (12)..(12): 2′-O-methyladenosine
  modified_base (13)..(13): gm
ucctggctcauag

SEQ ID NO: 423 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): cm
  modified_base (2)..(2): um
  modified_base (11)..(11): 2′-O-methyladenosine
  modified_base (12)..(12): um
  modified_base (13)..(13): 2′-O-methyladenosine
cucctggctcaua

SEQ ID NO: 424 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): um
  modified_base (2)..(2): cm
  modified_base (11)..(11): cm
  modified_base (12)..(12): 2′-O-methyladenosine
  modified_base (13)..(13): um
uctcctggctcau

SEQ ID NO: 425 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): cm
  modified_base (2)..(2): cm
  modified_base (11)..(11): gm
  modified_base (12)..(12): gm
  modified_base (13)..(13): 2′-O-methyladenosine
ccggcttgcagga

SEQ ID NO: 426 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): cm
  modified_base (2)..(2): cm
  modified_base (11)..(11): 2′-O-methyladenosine
  modified_base (12)..(12): gm
  modified_base (13)..(13): gm
cccggcttgcagg

SEQ ID NO: 427 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): gm
  modified_base (2)..(2): cm
  modified_base (11)..(11): cm
  modified_base (12)..(12): 2′-O-methyladenosine
  modified_base (13)..(13): gm
gcccggcttgcag

SEQ ID NO: 428 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): 2′-O-methyladenosine
  modified_base (2)..(2): gm
  modified_base (11)..(11): gm
  modified_base (12)..(12): cm
  modified_base (13)..(13): 2′-O-methyladenosine
agcccggcttgca

SEQ ID NO: 429 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): um
  modified_base (2)..(2): 2′-O-methyladenosine
  modified_base (11)..(11): um
  modified_base (12)..(12): gm
  modified_base (13)..(13): cm
uagcccggctugc

SEQ ID NO: 430 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): 2′-O-methyladenosine
  modified_base (2)..(2): um
  modified_base (11)..(11): um
  modified_base (12)..(12): um
  modified_base (13)..(13): gm
auagcccggcuug

SEQ ID NO: 431 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): cm
  modified_base (2)..(2): 2′-O-methyladenosine
  modified_base (11)..(11): cm
  modified_base (12)..(12): um
  modified_base (13)..(13): um
catagcccggcuu

SEQ ID NO: 432 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): 2′-O-methyladenosine
  modified_base (2)..(2): cm
  modified_base (11)..(11): cm
  modified_base (12)..(12): gm
  modified_base (13)..(13): gm
acacatagcccgg

SEQ ID NO: 433 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): gm
  modified_base (2)..(2): 2′-O-methyladenosine
  modified_base (11)..(11): cm
  modified_base (12)..(12): cm
  modified_base (13)..(13): gm
gacacatagcccg

SEQ ID NO: 434 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): um
  modified_base (2)..(2): gm
  modified_base (11)..(11): cm
  modified_base (12)..(12): 2′-O-methyladenosine
  modified_base (13)..(13): gm
ugtgagagggcag

SEQ ID NO: 435 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): um
  modified_base (2)..(2): um
  modified_base (11)..(11): 2′-O-methyladenosine
  modified_base (12)..(12): cm
  modified_base (13)..(13): 2′-O-methyladenosine
uugatgggccaca

SEQ ID NO: 436 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): gm
  modified_base (2)..(2): um
  modified_base (11)..(11): cm
  modified_base (12)..(12): 2′-O-methyladenosine
  modified_base (13)..(13): cm
gutgatgggccac

SEQ ID NO: 437 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): um
  modified_base (2)..(2): gm
  modified_base (11)..(11): cm
  modified_base (12)..(12): cm
  modified_base (13)..(13): 2′-O-methyladenosine
ugttgatgggcca

SEQ ID NO: 438 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): gm
  modified_base (2)..(2): um
  modified_base (11)..(11): gm
  modified_base (12)..(12): cm
  modified_base (13)..(13): cm
gugttgatgggcc

SEQ ID NO: 439 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): gm
  modified_base (2)..(2): gm
  modified_base (11)..(11): um
  modified_base (12)..(12): gm
  modified_base (13)..(13): gm
ggacatactcugg

SEQ ID NO: 440 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): 2′-O-methyladenosine
  modified_base (2)..(2): gm
  modified_base (11)..(11): cm
  modified_base (12)..(12): um
  modified_base (13)..(13): gm
aggacatactcug

SEQ ID NO: 441 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): gm
  modified_base (2)..(2): cm
  modified_base (11)..(11): cm
  modified_base (12)..(12): um
  modified_base (13)..(13): 2′-O-methyladenosine
gctccattttcua

SEQ ID NO: 442 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): gm
  modified_base (2)..(2): um
  modified_base (11)..(11): um
  modified_base (12)..(12): cm
  modified_base (13)..(13): 2′-O-methyladenosine
gutgggatatuca

SEQ ID NO: 443 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): gm
  modified_base (2)..(2): cm
  modified_base (11)..(11): gm
  modified_base (12)..(12): 2′-O-methyladenosine
  modified_base (13)..(13): 2′-O-methyladenosine
gcacttggcagaa

SEQ ID NO: 444 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): um
  modified_base (2)..(2): gm
  modified_base (11)..(11): 2′-O-methyladenosine
  modified_base (12)..(12): gm
  modified_base (13)..(13): 2′-O-methyladenosine
ugcacttggcaga

SEQ ID NO: 445 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): gm
  modified_base (2)..(2): um
  modified_base (11)..(11): cm
  modified_base (12)..(12): 2′-O-methyladenosine
  modified_base (13)..(13): gm
gugcacttggcag

SEQ ID NO: 446 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): 2′-O-methyladenosine
  modified_base (2)..(2): gm
  modified_base (11)..(11): gm
  modified_base (12)..(12): cm
  modified_base (13)..(13): 2′-O-methyladenosine
agtgcacttggca

SEQ ID NO: 447 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): cm
  modified_base (2)..(2): 2′-O-methyladenosine
  modified_base (11)..(11): gm
  modified_base (12)..(12): gm
  modified_base (13)..(13): cm
cagtgcacttggc

SEQ ID NO: 448 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): cm
  modified_base (2)..(2): um
  modified_base (11)..(11): um
  modified_base (12)..(12): um
  modified_base (13)..(13): gm
cucagtgcacuug

SEQ ID NO: 449 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): cm
  modified_base (2)..(2): cm
  modified_base (11)..(11): cm
  modified_base (12)..(12): um
  modified_base (13)..(13): um
cctcagtgcacuu

SEQ ID NO: 450 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): um
  modified_base (2)..(2): cm
  modified_base (11)..(11): 2′-O-methyladenosine
  modified_base (12)..(12): cm
  modified_base (13)..(13): um
ucctcagtgcacu

SEQ ID NO: 451 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): um
  modified_base (2)..(2): um
  modified_base (11)..(11): cm
  modified_base (12)..(12): 2′-O-methyladenosine
  modified_base (13)..(13): cm
uucctcagtgcac

SEQ ID NO: 452 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): cm
  modified_base (2)..(2): um
  modified_base (11)..(11): gm
  modified_base (12)..(12): cm
  modified_base (13)..(13): 2′-O-methyladenosine
cutcctcagtgca

SEQ ID NO: 453 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): cm
  modified_base (2)..(2): cm
  modified_base (11)..(11): um
  modified_base (12)..(12): gm
  modified_base (13)..(13): cm
ccttcctcagugc

SEQ ID NO: 454 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): um
  modified_base (2)..(2): cm
  modified_base (11)..(11): gm
  modified_base (12)..(12): um
  modified_base (13)..(13): gm
uccttcctcagug

SEQ ID NO: 455 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): gm
  modified_base (2)..(2): cm
  modified_base (11)..(11): um
  modified_base (12)..(12): 2′-O-methyladenosine
  modified_base (13)..(13): um
gctgatggctuau

SEQ ID NO: 456 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): 2′-O-methyladenosine
  modified_base (2)..(2): gm
  modified_base (11)..(11): um
  modified_base (12)..(12): um
  modified_base (13)..(13): 2′-O-methyladenosine
agctgatggcuua

SEQ ID NO: 457 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): cm
  modified_base (2)..(2): 2′-O-methyladenosine
  modified_base (11)..(11): cm
  modified_base (12)..(12): um
  modified_base (13)..(13): um
cagctgatggcuu

SEQ ID NO: 458 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): cm
  modified_base (2)..(2): cm
  modified_base (11)..(11): gm
  modified_base (12)..(12): cm
  modified_base (13)..(13): um
ccagctgatggcu

SEQ ID NO: 459 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): um
  modified_base (2)..(2): cm
  modified_base (11)..(11): gm
  modified_base (12)..(12): gm
  modified_base (13)..(13): cm
uccagctgatggc

SEQ ID NO: 460 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): um
  modified_base (2)..(2): um
  modified_base (11)..(11): um
  modified_base (12)..(12): gm
  modified_base (13)..(13): gm
uuccagctgaugg

SEQ ID NO: 461 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): gm
  modified_base (2)..(2): um
  modified_base (11)..(11): gm
  modified_base (12)..(12): 2′-O-methyladenosine
  modified_base (13)..(13): um
guttccagctgau

SEQ ID NO: 462 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): um
  modified_base (2)..(2): gm
  modified_base (11)..(11): um
  modified_base (12)..(12): gm
  modified_base (13)..(13): 2′-O-methyladenosine
ugtttccagcuga

SEQ ID NO: 463 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): um
  modified_base (2)..(2): um
  modified_base (11)..(11): cm
  modified_base (12)..(12): um
  modified_base (13)..(13): gm
uugtttccagcug

SEQ ID NO: 464 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): 2′-O-methyladenosine
  modified_base (2)..(2): 2′-O-methyladenosine
  modified_base (11)..(11): um
  modified_base (12)..(12): cm
  modified_base (13)..(13): cm
aactgctgtgucc

SEQ ID NO: 465 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): 2′-O-methyladenosine
  modified_base (2)..(2): 2′-O-methyladenosine
  modified_base (11)..(11): gm
  modified_base (12)..(12): um
  modified_base (13)..(13): gm
aaaaactgctgug

SEQ ID NO: 466 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): um
  modified_base (2)..(2): gm
  modified_base (11)..(11): um
  modified_base (12)..(12): cm
  modified_base (13)..(13): 2′-O-methyladenosine
uggcaaacatuca

SEQ ID NO: 467 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): gm
  modified_base (2)..(2): um
  modified_base (11)..(11): um
  modified_base (12)..(12): um
  modified_base (13)..(13): cm
guggcaaacauuc

SEQ ID NO: 468 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): gm
  modified_base (2)..(2): um
  modified_base (11)..(11): 2′-O-methyladenosine
  modified_base (12)..(12): cm
  modified_base (13)..(13): 2′-O-methyladenosine
gutgtggcaaaca

SEQ ID NO: 469 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): um
  modified_base (2)..(2): gm
  modified_base (11)..(11): 2′-O-methyladenosine
  modified_base (12)..(12): 2′-O-methyladenosine
  modified_base (13)..(13): cm
ugttgtggcaaac

SEQ ID NO: 470 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): gm
  modified_base (2)..(2): cm
  modified_base (11)..(11): gm
  modified_base (12)..(12): cm
  modified_base (13)..(13): 2′-O-methyladenosine
gcatgttgtggca

SEQ ID NO: 471 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): cm
  modified_base (2)..(2): gm
  modified_base (11)..(11): gm
  modified_base (12)..(12): gm
  modified_base (13)..(13): cm
cgcatgttgtggc

SEQ ID NO: 472 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): um
  modified_base (2)..(2): cm
  modified_base (11)..(11): um
  modified_base (12)..(12): gm
  modified_base (13)..(13): gm
ucgcatgttgugg

SEQ ID NO: 473 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): 2′-O-methyladenosine
  modified_base (2)..(2): um
  modified_base (11)..(11): gm
  modified_base (12)..(12): um
  modified_base (13)..(13): gm
aucgcatgttgug

SEQ ID NO: 474 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): 2′-O-methyladenosine
  modified_base (2)..(2): cm
  modified_base (11)..(11): um
  modified_base (12)..(12): um
  modified_base (13)..(13): gm
acatcgcatguug

SEQ ID NO: 475 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): um
  modified_base (2)..(2): um
  modified_base (11)..(11): cm
  modified_base (12)..(12): gm
  modified_base (13)..(13): cm
uuccaaacatcgc

SEQ ID NO: 476 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): 2′-O-methyladenosine
  modified_base (2)..(2): gm
  modified_base (11)..(11): cm
  modified_base (12)..(12): cm
  modified_base (13)..(13): 2′-O-methyladenosine
agtccaatttcca

SEQ ID NO: 477 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): cm
  modified_base (2)..(2): cm
  modified_base (11)..(11): um
  modified_base (12)..(12): gm
  modified_base (13)..(13): gm
ccctgcattcugg

SEQ ID NO: 478 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): um
  modified_base (2)..(2): cm
  modified_base (11)..(11): cm
  modified_base (12)..(12): um
  modified_base (13)..(13): gm
uccctgcattcug

SEQ ID NO: 479 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): um
  modified_base (2)..(2): um
  modified_base (11)..(11): um
  modified_base (12)..(12): cm
  modified_base (13)..(13): um
uuccctgcatucu

SEQ ID NO: 480 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): 2′-O-methyladenosine
  modified_base (2)..(2): um
  modified_base (11)..(11): cm
  modified_base (12)..(12): um
  modified_base (13)..(13): um
autgtctggtcuu

SEQ ID NO: 481 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): cm
  modified_base (2)..(2): 2′-O-methyladenosine
  modified_base (11)..(11): um
  modified_base (12)..(12): cm
  modified_base (13)..(13): um
cattgtctggucu

SEQ ID NO: 482 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): cm
  modified_base (2)..(2): cm
  modified_base (11)..(11): gm
  modified_base (12)..(12): um
  modified_base (13)..(13): cm
ccattgtctgguc

SEQ ID NO: 483 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): um
  modified_base (2)..(2): cm
  modified_base (11)..(11): gm
  modified_base (12)..(12): gm
  modified_base (13)..(13): um
uccattgtctggu

SEQ ID NO: 484 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): 2′-O-methyladenosine
  modified_base (2)..(2): um
  modified_base (11)..(11): um
  modified_base (12)..(12): gm
  modified_base (13)..(13): gm
auccattgtcugg

SEQ ID NO: 485 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): 2′-O-methyladenosine
  modified_base (2)..(2): 2′-O-methyladenosine
  modified_base (11)..(11): cm
  modified_base (12)..(12): um
  modified_base (13)..(13): gm
aatccattgtcug

SEQ ID NO: 486 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): gm
  modified_base (2)..(2): cm
  modified_base (11)..(11): um
  modified_base (12)..(12): cm
  modified_base (13)..(13): 2′-O-methyladenosine
gcaggatagtuca

SEQ ID NO: 487 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): gm
  modified_base (2)..(2): gm
  modified_base (11)..(11): gm
  modified_base (12)..(12): 2′-O-methyladenosine
  modified_base (13)..(13): um
ggttttgcaggau

SEQ ID NO: 488 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): um
  modified_base (2)..(2): gm
  modified_base (11)..(11): gm
  modified_base (12)..(12): gm
  modified_base (13)..(13): 2′-O-methyladenosine
uggttttgcagga

SEQ ID NO: 489 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): gm
  modified_base (2)..(2): gm
  modified_base (11)..(11): um
  modified_base (12)..(12): um
  modified_base (13)..(13): gm
ggctttatccuug

SEQ ID NO: 490 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): gm
  modified_base (2)..(2): um
  modified_base (11)..(11): cm
  modified_base (12)..(12): cm
  modified_base (13)..(13): um
guggctttatccu

SEQ ID NO: 491 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): cm
  modified_base (2)..(2): 2′-O-methyladenosine
  modified_base (11)..(11): gm
  modified_base (12)..(12): cm
  modified_base (13)..(13): cm
catcatggcagcc

SEQ ID NO: 492 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): cm
  modified_base (2)..(2): cm
  modified_base (11)..(11): 2′-O-methyladenosine
  modified_base (12)..(12): gm
  modified_base (13)..(13): cm
ccatcatggcagc

SEQ ID NO: 493 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): um
  modified_base (2)..(2): cm
  modified_base (11)..(11): cm
  modified_base (12)..(12): 2′-O-methyladenosine
  modified_base (13)..(13): gm
uccatcatggcag

SEQ ID NO: 494 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): 2′-O-methyladenosine
  modified_base (2)..(2): um
  modified_base (11)..(11): gm
  modified_base (12)..(12): cm
  modified_base (13)..(13): 2′-O-methyladenosine
auccatcatggca

SEQ ID NO: 495 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): um
  modified_base (2)..(2): 2′-O-methyladenosine
  modified_base (11)..(11): gm
  modified_base (12)..(12): gm
  modified_base (13)..(13): cm
uatccatcatggc

SEQ ID NO: 496 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): 2′-O-methyladenosine
  modified_base (2)..(2): um
  modified_base (11)..(11): um
  modified_base (12)..(12): gm
  modified_base (13)..(13): gm
auatccatcaugg

SEQ ID NO: 497 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): cm
  modified_base (2)..(2): cm
  modified_base (11)..(11): um
  modified_base (12)..(12): 2′-O-methyladenosine
  modified_base (13)..(13): cm
cccagtttgguac

SEQ ID NO: 498 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): um
  modified_base (2)..(2): cm
  modified_base (11)..(11): gm
  modified_base (12)..(12): um
  modified_base (13)..(13): 2′-O-methyladenosine
ucccagtttggua

SEQ ID NO: 499 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): um
  modified_base (2)..(2): um
  modified_base (11)..(11): gm
  modified_base (12)..(12): gm
  modified_base (13)..(13): um
uucccagtttggu

SEQ ID NO: 500 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): um
  modified_base (2)..(2): um
  modified_base (11)..(11): um
  modified_base (12)..(12): gm
  modified_base (13)..(13): gm
uutcccagttugg

SEQ ID NO: 501 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): gm
  modified_base (2)..(2): um
  modified_base (11)..(11): um
  modified_base (12)..(12): um
  modified_base (13)..(13): gm
guttcccagtuug

SEQ ID NO: 502 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): cm
  modified_base (2)..(2): 2′-O-methyladenosine
  modified_base (11)..(11): gm
  modified_base (12)..(12): um
  modified_base (13)..(13): um
cagtttcccaguu

SEQ ID NO: 503 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): cm
  modified_base (2)..(2): cm
  modified_base (11)..(11): 2′-O-methyladenosine
  modified_base (12)..(12): gm
  modified_base (13)..(13): um
ccagtttcccagu

SEQ ID NO: 504 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): 2′-O-methyladenosine
  modified_base (2)..(2): cm
  modified_base (11)..(11): cm
  modified_base (12)..(12): 2′-O-methyladenosine
  modified_base (13)..(13): gm
accagtttcccag

SEQ ID NO: 505 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): gm
  modified_base (2)..(2): 2′-O-methyladenosine
  modified_base (11)..(11): cm
  modified_base (12)..(12): cm
  modified_base (13)..(13): 2′-O-methyladenosine
gaccagtttccca

SEQ ID NO: 506 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): 2′-O-methyladenosine
  modified_base (2)..(2): gm
  modified_base (11)..(11): cm
  modified_base (12)..(12): cm
  modified_base (13)..(13): cm
agaccagtttccc

SEQ ID NO: 507 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): gm
  modified_base (2)..(2): cm
  modified_base (11)..(11): um
  modified_base (12)..(12): um
  modified_base (13)..(13): cm
gcagaccagtuuc

SEQ ID NO: 508 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): gm
  modified_base (2)..(2): gm
  modified_base (11)..(11): um
  modified_base (12)..(12): um
  modified_base (13)..(13): um
ggcagaccaguuu

SEQ ID NO: 509 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): um
  modified_base (2)..(2): gm
  modified_base (11)..(11): gm
  modified_base (12)..(12): um
  modified_base (13)..(13): um
uggcagaccaguu

SEQ ID NO: 510 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): 2′-O-methyladenosine
  modified_base (2)..(2): um
  modified_base (11)..(11): 2′-O-methyladenosine
  modified_base (12)..(12): gm
  modified_base (13)..(13): um
auggcagaccagu

SEQ ID NO: 511 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): cm
  modified_base (2)..(2): 2′-O-methyladenosine
  modified_base (11)..(11): cm
  modified_base (12)..(12): 2′-O-methyladenosine
  modified_base (13)..(13): gm
catggcagaccag

SEQ ID NO: 512 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): gm
  modified_base (2)..(2): cm
  modified_base (11)..(11): cm
  modified_base (12)..(12): cm
  modified_base (13)..(13): 2′-O-methyladenosine
gcatggcagacca

SEQ ID NO: 513 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): gm
  modified_base (2)..(2): gm
  modified_base (11)..(11): 2′-O-methyladenosine
  modified_base (12)..(12): cm
  modified_base (13)..(13): cm
ggcatggcagacc

SEQ ID NO: 514 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): um
  modified_base (2)..(2): gm
  modified_base (11)..(11): gm
  modified_base (12)..(12): 2′-O-methyladenosine
  modified_base (13)..(13): cm
uggcatggcagac

SEQ ID NO: 515 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): um
  modified_base (2)..(2): um
  modified_base (11)..(11): 2′-O-methyladenosine
  modified_base (12)..(12): gm
  modified_base (13)..(13): 2′-O-methyladenosine
uuggcatggcaga

SEQ ID NO: 516 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): cm
  modified_base (2)..(2): um
  modified_base (11)..(11): cm
  modified_base (12)..(12): 2′-O-methyladenosine
  modified_base (13)..(13): gm
cutggcatggcag

SEQ ID NO: 517 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): 2′-O-methyladenosine
  modified_base (2)..(2): cm
  modified_base (11)..(11): gm
  modified_base (12)..(12): cm
  modified_base (13)..(13): 2′-O-methyladenosine
acttggcatggca

SEQ ID NO: 518 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): 2′-O-methyladenosine
  modified_base (2)..(2): 2′-O-methyladenosine
  modified_base (11)..(11): gm
  modified_base (12)..(12): gm
  modified_base (13)..(13): cm
aacttggcatggc

SEQ ID NO: 519 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): cm
  modified_base (2)..(2): 2′-O-methyladenosine
  modified_base (11)..(11): um
  modified_base (12)..(12): gm
  modified_base (13)..(13): gm
caacttggcaugg

SEQ ID NO: 520 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): 2′-O-methyladenosine
  modified_base (2)..(2): cm
  modified_base (11)..(11): 2′-O-methyladenosine
  modified_base (12)..(12): um
  modified_base (13)..(13): gm
acaacttggcaug

SEQ ID NO: 521 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): gm
  modified_base (2)..(2): cm
  modified_base (11)..(11): um
  modified_base (12)..(12): um
  modified_base (13)..(13): gm
gctttacaacuug

SEQ ID NO: 522 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): um
  modified_base (2)..(2): cm
  modified_base (11)..(11): um
  modified_base (12)..(12): um
  modified_base (13)..(13): um
ucacaggtacuuu

SEQ ID NO: 523 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): 2′-O-methyladenosine
  modified_base (2)..(2): cm
  modified_base (11)..(11): gm
  modified_base (12)..(12): gm
  modified_base (13)..(13): cm
acaccacagtggc

SEQ ID NO: 524 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): gm
  modified_base (2)..(2): um
  modified_base (11)..(11): gm
  modified_base (12)..(12): um
  modified_base (13)..(13): gm
guacaccacagug

SEQ ID NO: 525 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): gm
  modified_base (2)..(2): gm
  modified_base (11)..(11): 2′-O-methyladenosine
  modified_base (12)..(12): gm
  modified_base (13)..(13): um
ggtacaccacagu

SEQ ID NO: 526 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): um
  modified_base (2)..(2): gm
  modified_base (11)..(11): cm
  modified_base (12)..(12): 2′-O-methyladenosine
  modified_base (13)..(13): gm
uggtacaccacag

SEQ ID NO: 527 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): um
  modified_base (2)..(2): um
  modified_base (11)..(11): 2′-O-methyladenosine
  modified_base (12)..(12): cm
  modified_base (13)..(13): 2′-O-methyladenosine
uuggtacaccaca

SEQ ID NO: 528 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): cm
  modified_base (2)..(2): um
  modified_base (11)..(11): cm
  modified_base (12)..(12): 2′-O-methyladenosine
  modified_base (13)..(13): cm
cutggtacaccac

SEQ ID NO: 529 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): cm
  modified_base (2)..(2): cm
  modified_base (11)..(11): cm
  modified_base (12)..(12): cm
  modified_base (13)..(13): 2′-O-methyladenosine
ccttggtacacca

SEQ ID NO: 530 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): um
  modified_base (2)..(2): cm
  modified_base (11)..(11): 2′-O-methyladenosine
  modified_base (12)..(12): cm
  modified_base (13)..(13): cm
uccttggtacacc

SEQ ID NO: 531 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): cm
  modified_base (2)..(2): um
  modified_base (11)..(11): cm
  modified_base (12)..(12): 2′-O-methyladenosine
  modified_base (13)..(13): cm
cuccttggtacac

SEQ ID NO: 532 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): um
  modified_base (2)..(2): cm
  modified_base (11)..(11): 2′-O-methyladenosine
  modified_base (12)..(12): cm
  modified_base (13)..(13): 2′-O-methyladenosine
uctccttggtaca

SEQ ID NO: 533 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): cm
  modified_base (2)..(2): um
  modified_base (11)..(11): um
  modified_base (12)..(12): 2′-O-methyladenosine
  modified_base (13)..(13): cm
cuctccttgguac

SEQ ID NO: 534 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): um
  modified_base (2)..(2): cm
  modified_base (11)..(11): gm
  modified_base (12)..(12): um
  modified_base (13)..(13): 2′-O-methyladenosine
uctctccttggua

SEQ ID NO: 535 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): cm
  modified_base (2)..(2): um
  modified_base (11)..(11): gm
  modified_base (12)..(12): gm
  modified_base (13)..(13): um
cuctctccttggu

SEQ ID NO: 536 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): um
  modified_base (2)..(2): cm
  modified_base (11)..(11): um
  modified_base (12)..(12): gm
  modified_base (13)..(13): gm
uctctctcctugg

SEQ ID NO: 537 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): cm
  modified_base (2)..(2): um
  modified_base (11)..(11): um
  modified_base (12)..(12): um
  modified_base (13)..(13): gm
cuctctctccuug

SEQ ID NO: 538 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): 2′-O-methyladenosine
  modified_base (2)..(2): cm
  modified_base (11)..(11): cm
  modified_base (12)..(12): um
  modified_base (13)..(13): um
actctctctccuu

SEQ ID NO: 539 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): um
  modified_base (2)..(2): 2′-O-methyladenosine
  modified_base (11)..(11): cm
  modified_base (12)..(12): cm
  modified_base (13)..(13): um
uactctctctccu

SEQ ID NO: 540 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): um
  modified_base (2)..(2): um
  modified_base (11)..(11): um
  modified_base (12)..(12): cm
  modified_base (13)..(13): cm
uuactctctcucc

SEQ ID NO: 541 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): gm
  modified_base (2)..(2): cm
  modified_base (11)..(11): cm
  modified_base (12)..(12): um
  modified_base (13)..(13): um
gcattccattcuu

SEQ ID NO: 542 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): 2′-O-methyladenosine
  modified_base (2)..(2): gm
  modified_base (11)..(11): um
  modified_base (12)..(12): cm
  modified_base (13)..(13): um
agcattccatucu

SEQ ID NO: 543 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): 2′-O-methyladenosine
  modified_base (2)..(2): gm
  modified_base (11)..(11): cm
  modified_base (12)..(12): um
  modified_base (13)..(13): um
agctacacttcuu

SEQ ID NO: 544 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): um
  modified_base (2)..(2): 2′-O-methyladenosine
  modified_base (11)..(11): um
  modified_base (12)..(12): cm
  modified_base (13)..(13): um
uagctacactucu

SEQ ID NO: 545 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): um
  modified_base (2)..(2): um
  modified_base (11)..(11): um
  modified_base (12)..(12): gm
  modified_base (13)..(13): gm
uugaagcattugg

SEQ ID NO: 546 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): gm
  modified_base (2)..(2): um
  modified_base (11)..(11): gm
  modified_base (12)..(12): cm
  modified_base (13)..(13): 2′-O-methyladenosine
gutccttgaagca

SEQ ID NO: 547 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): um
  modified_base (2)..(2): gm
  modified_base (11)..(11): 2′-O-methyladenosine
  modified_base (12)..(12): gm
  modified_base (13)..(13): cm
ugttccttgaagc

SEQ ID NO: 548 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): gm
  modified_base (2)..(2): um
  modified_base (11)..(11): 2′-O-methyladenosine
  modified_base (12)..(12): 2′-O-methyladenosine
  modified_base (13)..(13): gm
gugttccttgaag

SEQ ID NO: 549 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): um
  modified_base (2)..(2): gm
  modified_base (11)..(11): gm
  modified_base (12)..(12): 2′-O-methyladenosine
  modified_base (13)..(13): 2′-O-methyladenosine
ugtgttccttgaa

SEQ ID NO: 550 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): cm
  modified_base (2)..(2): um
  modified_base (11)..(11): um
  modified_base (12)..(12): gm
  modified_base (13)..(13): 2′-O-methyladenosine
cugtgttcctuga

SEQ ID NO: 551 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): 2′-O-methyladenosine
  modified_base (2)..(2): cm
  modified_base (11)..(11): um
  modified_base (12)..(12): um
  modified_base (13)..(13): gm
actgtgttccuug

SEQ ID NO: 552 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): 2′-O-methyladenosine
  modified_base (2)..(2): 2′-O-methyladenosine
  modified_base (11)..(11): cm
  modified_base (12)..(12): um
  modified_base (13)..(13): um
aactgtgttccuu

SEQ ID NO: 553 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): gm
  modified_base (2)..(2): 2′-O-methyladenosine
  modified_base (11)..(11): cm
  modified_base (12)..(12): cm
  modified_base (13)..(13): um
gaactgtgttccu

SEQ ID NO: 554 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): 2′-O-methyladenosine
  modified_base (2)..(2): gm
  modified_base (11)..(11): um
  modified_base (12)..(12): cm
  modified_base (13)..(13): cm
agaactgtgtucc

SEQ ID NO: 555 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): gm
  modified_base (2)..(2): 2′-O-methyladenosine
  modified_base (11)..(11): um
  modified_base (12)..(12): um
  modified_base (13)..(13): cm
gagaactgtguuc

SEQ ID NO: 556 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): 2′-O-methyladenosine
  modified_base (2)..(2): gm
  modified_base (11)..(11): gm
  modified_base (12)..(12): um
  modified_base (13)..(13): um
agagaactgtguu

SEQ ID NO: 557 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): cm
  modified_base (2)..(2): 2′-O-methyladenosine
  modified_base (11)..(11): um
  modified_base (12)..(12): gm
  modified_base (13)..(13): um
cagagaactgugu

SEQ ID NO: 558 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): cm
  modified_base (2)..(2): cm
  modified_base (11)..(11): gm
  modified_base (12)..(12): um
  modified_base (13)..(13): gm
ccagagaactgug

SEQ ID NO: 559 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): gm
  modified_base (2)..(2): cm
  modified_base (11)..(11): um
  modified_base (12)..(12): gm
  modified_base (13)..(13): um
gccagagaacugu

SEQ ID NO: 560 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): 2′-O-methyladenosine
  modified_base (2)..(2): gm
  modified_base (11)..(11): cm
  modified_base (12)..(12): um
  modified_base (13)..(13): gm
agccagagaacug

SEQ ID NO: 561 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): 2′-O-methyladenosine
  modified_base (2)..(2): 2′-O-methyladenosine
  modified_base (11)..(11): 2′-O-methyladenosine
  modified_base (12)..(12): cm
  modified_base (13)..(13): um
aagccagagaacu

SEQ ID NO: 562 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): gm
  modified_base (2)..(2): cm
  modified_base (11)..(11): um
  modified_base (12)..(12): cm
  modified_base (13)..(13): cm
gcatcagtttucc

SEQ ID NO: 563 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): um
  modified_base (2)..(2): gm
  modified_base (11)..(11): um
  modified_base (12)..(12): um
  modified_base (13)..(13): cm
ugcatcagttuuc

SEQ ID NO: 564 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): gm
  modified_base (2)..(2): 2′-O-methyladenosine
  modified_base (11)..(11): um
  modified_base (12)..(12): um
  modified_base (13)..(13): um
gatgcatcaguuu

SEQ ID NO: 565 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): gm
  modified_base (2)..(2): gm
  modified_base (11)..(11): gm
  modified_base (12)..(12): um
  modified_base (13)..(13): um
ggatgcatcaguu

SEQ ID NO: 566 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): cm
  modified_base (2)..(2): gm
  modified_base (11)..(11): 2′-O-methyladenosine
  modified_base (12)..(12): gm
  modified_base (13)..(13): um
cggatgcatcagu

SEQ ID NO: 567 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): um
  modified_base (2)..(2): cm
  modified_base (11)..(11): cm
  modified_base (12)..(12): 2′-O-methyladenosine
  modified_base (13)..(13): gm
ucggatgcatcag

SEQ ID NO: 568 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): 2′-O-methyladenosine
  modified_base (2)..(2): um
  modified_base (11)..(11): um
  modified_base (12)..(12): cm
  modified_base (13)..(13): 2′-O-methyladenosine
aucggatgcauca

SEQ ID NO: 569 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): cm
  modified_base (2)..(2): 2′-O-methyladenosine
  modified_base (11)..(11): 2′-O-methyladenosine
  modified_base (12)..(12): um
  modified_base (13)..(13): cm
catcggatgcauc

SEQ ID NO: 570 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): 2′-O-methyladenosine
  modified_base (2)..(2): cm
  modified_base (11)..(11): cm
  modified_base (12)..(12): 2′-O-methyladenosine
  modified_base (13)..(13): um
acatcggatgcau

SEQ ID NO: 571 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): um
  modified_base (2)..(2): 2′-O-methyladenosine
  modified_base (11)..(11): gm
  modified_base (12)..(12): cm
  modified_base (13)..(13): 2′-O-methyladenosine
uacatcggatgca

SEQ ID NO: 572 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): um
  modified_base (2)..(2): um
  modified_base (11)..(11): um
  modified_base (12)..(12): gm
  modified_base (13)..(13): cm
uuacatcggaugc

SEQ ID NO: 573 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): gm
  modified_base (2)..(2): cm
  modified_base (11)..(11): gm
  modified_base (12)..(12): gm
  modified_base (13)..(13): 2′-O-methyladenosine
gctttacatcgga

SEQ ID NO: 574 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): gm
  modified_base (2)..(2): gm
  modified_base (11)..(11): cm
  modified_base (12)..(12): gm
  modified_base (13)..(13): gm
ggctttacatcgg

SEQ ID NO: 575 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): um
  modified_base (2)..(2): gm
  modified_base (11)..(11): um
  modified_base (12)..(12): cm
  modified_base (13)..(13): gm
uggctttacaucg

SEQ ID NO: 576 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): um
  modified_base (2)..(2): gm
  modified_base (11)..(11): gm
  modified_base (12)..(12): 2′-O-methyladenosine
  modified_base (13)..(13): 2′-O-methyladenosine
ugtggaatctgaa

SEQ ID NO: 577 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): 2′-O-methyladenosine
  modified_base (2)..(2): gm
  modified_base (11)..(11): um
  modified_base (12)..(12): um
  modified_base (13)..(13): um
agtgtgacatuuu

SEQ ID NO: 578 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): 2′-O-methyladenosine
  modified_base (2)..(2): 2′-O-methyladenosine
  modified_base (11)..(11): um
  modified_base (12)..(12): um
  modified_base (13)..(13): um
aagtgtgacauuu

SEQ ID NO: 579 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): cm
  modified_base (2)..(2): cm
  modified_base (11)..(11): 2′-O-methyladenosine
  modified_base (12)..(12): cm
  modified_base (13)..(13): 2′-O-methyladenosine
ccttggatgaaca

SEQ ID NO: 580 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): um
  modified_base (2)..(2): cm
  modified_base (11)..(11): 2′-O-methyladenosine
  modified_base (12)..(12): 2′-O-methyladenosine
  modified_base (13)..(13): cm
uccttggatgaac

SEQ ID NO: 581 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): gm
  modified_base (2)..(2): um
  modified_base (11)..(11): um
  modified_base (12)..(12): gm
  modified_base (13)..(13): 2′-O-methyladenosine
gutccttggauga

SEQ ID NO: 582 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): gm
  modified_base (2)..(2): gm
  modified_base (11)..(11): 2′-O-methyladenosine
  modified_base (12)..(12): um
  modified_base (13)..(13): gm
ggttccttggaug

SEQ ID NO: 583 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): 2′-O-methyladenosine
  modified_base (2)..(2): gm
  modified_base (11)..(11): gm
  modified_base (12)..(12): 2′-O-methyladenosine
  modified_base (13)..(13): um
aggttccttggau

SEQ ID NO: 584 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): um
  modified_base (2)..(2): 2′-O-methyladenosine
  modified_base (11)..(11): gm
  modified_base (12)..(12): gm
  modified_base (13)..(13): 2′-O-methyladenosine
uaggttccttgga

SEQ ID NO: 585 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): um
  modified_base (2)..(2): um
  modified_base (11)..(11): um
  modified_base (12)..(12): gm
  modified_base (13)..(13): gm
uuaggttcctugg

SEQ ID NO: 586 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): 2 ′-O,4 ′-C-methylene- linked bicyclic ribonucleotide
  modified_base (2)..(2): 2 ′-O,4 ′-C-methylene- linked bicyclic ribonucleotide
  modified_base (11)..(11): 2 ′-O,4 ′-C-methylene- linked bicyclic ribonucleotide
  modified_base (12)..(12): 5-methylcytosine; 2 ′-O,4 ′-C-methylene- linked bicyclic ribonucleotide
  modified_base (13)..(13): 2 ′-O,4 ′-C-methylene- linked bicyclic ribonucleotide
tagcaacatggca

SEQ ID NO: 587 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): 2 ′-O,4 ′-C-methylene- linked bicyclic ribonucleotide
  modified_base (2)..(2): 2 ′-O,4 ′-C-methylene- linked bicyclic ribonucleotide
  modified_base (11)..(11): 2 ′-O,4 ′-C-methylene- linked bicyclic ribonucleotide
  modified_base (12)..(12): 2 ′-O,4 ′-C-methylene- linked bicyclic ribonucleotide
  modified_base (13)..(13): 5-methylcytosine; 2 ′-O,4 ′-C-methylene- linked bicyclic ribonucleotide
atagcaacatggc

SEQ ID NO: 588 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): 5-methylcytosine; 2 ′-O,4 ′-C-methylene- linked bicyclic ribonucleotide
  modified_base (2)..(2): 2 ′-O,4 ′-C-methylene- linked bicyclic ribonucleotide
  modified_base (11)..(11): 2 ′-O,4 ′-C-methylene- linked bicyclic ribonucleotide
  modified_base (12)..(12): 2 ′-O,4 ′-C-methylene- linked bicyclic ribonucleotide
  modified_base (13)..(13): 2 ′-O,4 ′-C-methylene- linked bicyclic ribonucleotide
caatagcaacatg

SEQ ID NO: 589 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): 2 ′-O,4 ′-C-methylene- linked bicyclic ribonucleotide
  modified_base (2)..(2): 5-methylcytosine; 2 ′-O,4 ′-C-methylene- linked bicyclic ribonucleotide
  modified_base (11)..(11): 5-methylcytosine; 2 ′-O,4 ′-C-methylene- linked bicyclic ribonucleotide
  modified_base (12)..(12): 2 ′-O,4 ′-C-methylene- linked bicyclic ribonucleotide
  modified_base (13)..(13): 2 ′-O,4 ′-C-methylene- linked bicyclic ribonucleotide
gcaatagcaacat

SEQ ID NO: 590 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): 5-methylcytosine; 2 ′-O,4 ′-C-methylene- linked bicyclic ribonucleotide
  modified_base (2)..(2): 5-methylcytosine; 2 ′-O,4 ′-C-methylene- linked bicyclic ribonucleotide
  modified_base (11)..(11): 5-methylcytosine; 2 ′-O,4 ′-C-methylene- linked bicyclic ribonucleotide
  modified_base (12)..(12): 2 ′-O,4 ′-C-methylene- linked bicyclic ribonucleotide
  modified_base (13)..(13): 2 ′-O,4 ′-C-methylene- linked bicyclic ribonucleotide
cctgcaatagcaa

SEQ ID NO: 591 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): 2 ′-O,4 ′-C-methylene- linked bicyclic ribonucleotide
  modified_base (2)..(2): 5-methylcytosine; 2 ′-O,4 ′-C-methylene- linked bicyclic ribonucleotide
  modified_base (11)..(11): 2 ′-O,4 ′-C-methylene- linked bicyclic ribonucleotide
  modified_base (12)..(12): 5-methylcytosine; 2 ′-O,4 ′-C-methylene- linked bicyclic ribonucleotide
  modified_base (13)..(13): 2 ′-O,4 ′-C-methylene- linked bicyclic ribonucleotide
tcctgcaatagca

SEQ ID NO: 592 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): 2 ′-O,4 ′-C-methylene- linked bicyclic ribonucleotide
  modified_base (2)..(2): 2 ′-O,4 ′-C-methylene- linked bicyclic ribonucleotide
  modified_base (11)..(11): 2 ′-O,4 ′-C-methylene- linked bicyclic ribonucleotide
  modified_base (12)..(12): 2 ′-O,4 ′-C-methylene- linked bicyclic ribonucleotide
  modified_base (13)..(13): 5-methylcytosine; 2 ′-O,4 ′-C-methylene- linked bicyclic ribonucleotide
gtcctgcaatagc

SEQ ID NO: 593 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): 2 ′-O,4 ′-C-methylene- linked bicyclic ribonucleotide
  modified_base (2)..(2): 5-methylcytosine; 2 ′-O,4 ′-C-methylene- linked bicyclic ribonucleotide
  modified_base (11)..(11): 2 ′-O,4 ′-C-methylene- linked bicyclic ribonucleotide
  modified_base (12)..(12): 2 ′-O,4 ′-C-methylene- linked bicyclic ribonucleotide
  modified_base (13)..(13): 2 ′-O,4 ′-C-methylene- linked bicyclic ribonucleotide
tccgtcctgcaat

SEQ ID NO: 594 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): 2 ′-O,4 ′-C-methylene- linked bicyclic ribonucleotide
  modified_base (2)..(2): 2 ′-O,4 ′-C-methylene- linked bicyclic ribonucleotide
  modified_base (11)..(11): 5-methylcytosine; 2 ′-O,4 ′-C-methylene- linked bicyclic ribonucleotide
  modified_base (12)..(12): 2 ′-O,4 ′-C-methylene- linked bicyclic ribonucleotide
  modified_base (13)..(13): 2 ′-O,4 ′-C-methylene- linked bicyclic ribonucleotide
gtccgtcctgcaa

SEQ ID NO: 595 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): 2 ′-O,4 ′-C-methylene- linked bicyclic ribonucleotide
  modified_base (2)..(2): 2 ′-O,4 ′-C-methylene- linked bicyclic ribonucleotide
  modified_base (11)..(11): 2 ′-O,4 ′-C-methylene- linked bicyclic ribonucleotide
  modified_base (12)..(12): 5-methylcytosine; 2 ′-O,4 ′-C-methylene- linked bicyclic ribonucleotide
  modified_base (13)..(13): 2 ′-O,4 ′-C-methylene- linked bicyclic ribonucleotide
ggtccgtcctgca

SEQ ID NO: 596 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): 5-methylcytosine; 2 ′-O,4 ′-C-methylene- linked bicyclic ribonucleotide
  modified_base (2)..(2): 2 ′-O,4 ′-C-methylene- linked bicyclic ribonucleotide
  modified_base (11)..(11): 2 ′-O,4 ′-C-methylene- linked bicyclic ribonucleotide
  modified_base (12)..(12): 5-methylcytosine; 2 ′-O,4 ′-C-methylene- linked bicyclic ribonucleotide
  modified_base (13)..(13): 2 ′-O,4 ′-C-methylene- linked bicyclic ribonucleotide
ctggcttgggaca

SEQ ID NO: 597 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): 2 ′-O,4 ′-C-methylene- linked bicyclic ribonucleotide
  modified_base (2)..(2): 5-methylcytosine; 2 ′-O,4 ′-C-methylene- linked bicyclic ribonucleotide
  modified_base (11)..(11): 2 ′-O,4 ′-C-methylene- linked bicyclic ribonucleotide
  modified_base (12)..(12): 2 ′-O,4 ′-C-methylene- linked bicyclic ribonucleotide
  modified_base (13)..(13): 2 ′-O,4 ′-C-methylene- linked bicyclic ribonucleotide
tcatctggcttgg

SEQ ID NO: 598 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): 2 ′-O,4 ′-C-methylene- linked bicyclic ribonucleotide
  modified_base (2)..(2): 5-methylcytosine; 2 ′-O,4 ′-C-methylene- linked bicyclic ribonucleotide
  modified_base (11)..(11): 5-methylcytosine; 2 ′-O,4 ′-C-methylene- linked bicyclic ribonucleotide
  modified_base (12)..(12): 2 ′-O,4 ′-C-methylene- linked bicyclic ribonucleotide
  modified_base (13)..(13): 2 ′-O,4 ′-C-methylene- linked bicyclic ribonucleotide
tctcctggctcat

SEQ ID NO: 599 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): 2 ′-O,4 ′-C-methylene- linked bicyclic ribonucleotide
  modified_base (2)..(2): 2 ′-O,4 ′-C-methylene- linked bicyclic ribonucleotide
  modified_base (11)..(11): 2 ′-O,4 ′-C-methylene- linked bicyclic ribonucleotide
  modified_base (12)..(12): 5-methylcytosine; 2 ′-O,4 ′-C-methylene- linked bicyclic ribonucleotide
  modified_base (13)..(13): 2 ′-O,4 ′-C-methylene- linked bicyclic ribonucleotide
agcccggcttgca

SEQ ID NO: 600 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): 2 ′-O,4 ′-C-methylene- linked bicyclic ribonucleotide
  modified_base (2)..(2): 2 ′-O,4 ′-C-methylene- linked bicyclic ribonucleotide
  modified_base (11)..(11): 2 ′-O,4 ′-C-methylene- linked bicyclic ribonucleotide
  modified_base (12)..(12): 2 ′-O,4 ′-C-methylene- linked bicyclic ribonucleotide
  modified_base (13)..(13): 5-methylcytosine; 2 ′-O,4 ′-C-methylene- linked bicyclic ribonucleotide
tagcccggcttgc

SEQ ID NO: 601 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): 2 ′-O,4 ′-C-methylene- linked bicyclic ribonucleotide
  modified_base (2)..(2): 2 ′-O,4 ′-C-methylene- linked bicyclic ribonucleotide
  modified_base (11)..(11): 2 ′-O,4 ′-C-methylene- linked bicyclic ribonucleotide
  modified_base (12)..(12): 2 ′-O,4 ′-C-methylene- linked bicyclic ribonucleotide
  modified_base (13)..(13): 2 ′-O,4 ′-C-methylene- linked bicyclic ribonucleotide
atagcccggcttg

SEQ ID NO: 602 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): 2 ′-O,4 ′-C-methylene- linked bicyclic ribonucleotide
  modified_base (2)..(2): 2 ′-O,4 ′-C-methylene- linked bicyclic ribonucleotide
  modified_base (11)..(11): 2 ′-O,4 ′-C-methylene- linked bicyclic ribonucleotide
  modified_base (12)..(12): 5-methylcytosine; 2 ′-O,4 ′-C-methylene- linked bicyclic ribonucleotide
  modified_base (13)..(13): 5-methylcytosine; 2 ′-O,4 ′-C-methylene- linked bicyclic ribonucleotide
gtgttgatgggcc

SEQ ID NO: 603 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): 2 ′-O,4 ′-C-methylene- linked bicyclic ribonucleotide
  modified_base (2)..(2): 2 ′-O,4 ′-C-methylene- linked bicyclic ribonucleotide
  modified_base (11)..(11): 2 ′-O,4 ′-C-methylene- linked bicyclic ribonucleotide
  modified_base (12)..(12): 2 ′-O,4 ′-C-methylene- linked bicyclic ribonucleotide
  modified_base (13)..(13): 2 ′-O,4 ′-C-methylene- linked bicyclic ribonucleotide
ggacatactctgg

SEQ ID NO: 604 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): 2 ′-O,4 ′-C-methylene- linked bicyclic ribonucleotide
  modified_base (2)..(2): 2 ′-O,4 ′-C-methylene- linked bicyclic ribonucleotide
  modified_base (11)..(11): 2 ′-O,4 ′-C-methylene- linked bicyclic ribonucleotide
  modified_base (12)..(12): 5-methylcytosine; 2 ′-O,4 ′-C-methylene- linked bicyclic ribonucleotide
  modified_base (13)..(13): 2 ′-O,4 ′-C-methylene- linked bicyclic ribonucleotide
aaggacatactct

SEQ ID NO: 605 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): 2 ′-O,4 ′-C-methylene- linked bicyclic ribonucleotide
  modified_base (2)..(2): 2 ′-O,4 ′-C-methylene- linked bicyclic ribonucleotide
  modified_base (11)..(11): 5-methylcytosine; 2 ′-O,4 ′-C-methylene- linked bicyclic ribonucleotide
  modified_base (12)..(12): 2 ′-O,4 ′-C-methylene- linked bicyclic ribonucleotide
  modified_base (13)..(13): 5-methylcytosine; 2 ′-O,4 ′-C-methylene- linked bicyclic ribonucleotide
aaaggacatactc

SEQ ID NO: 606 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): 2 ′-O,4 ′-C-methylene- linked bicyclic ribonucleotide
  modified_base (2)..(2): 2 ′-O,4 ′-C-methylene- linked bicyclic ribonucleotide
  modified_base (11)..(11): 2 ′-O,4 ′-C-methylene- linked bicyclic ribonucleotide
  modified_base (12)..(12): 2 ′-O,4 ′-C-methylene- linked bicyclic ribonucleotide
  modified_base (13)..(13): 2 ′-O,4 ′-C-methylene- linked bicyclic ribonucleotide
gggatattcaaaa

SEQ ID NO: 607 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): 2 ′-O,4 ′-C-methylene- linked bicyclic ribonucleotide
  modified_base (2)..(2): 2 ′-O,4 ′-C-methylene- linked bicyclic ribonucleotide
  modified_base (11)..(11): 2 ′-O,4 ′-C-methylene- linked bicyclic ribonucleotide
  modified_base (12)..(12): 2 ′-O,4 ′-C-methylene- linked bicyclic ribonucleotide
  modified_base (13)..(13): 2 ′-O,4 ′-C-methylene- linked bicyclic ribonucleotide
tgggatattcaaa

SEQ ID NO: 608 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): 2 ′-O,4 ′-C-methylene- linked bicyclic ribonucleotide
  modified_base (2)..(2): 2 ′-O,4 ′-C-methylene- linked bicyclic ribonucleotide
  modified_base (11)..(11): 5-methylcytosine; 2 ′-O,4 ′-C-methylene- linked bicyclic ribonucleotide
  modified_base (12)..(12): 2 ′-O,4 ′-C-methylene- linked bicyclic ribonucleotide
  modified_base (13)..(13): 2 ′-O,4 ′-C-methylene- linked bicyclic ribonucleotide
ttgggatattcaa

SEQ ID NO: 609 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): 2 ′-O,4 ′-C-methylene- linked bicyclic ribonucleotide
  modified_base (2)..(2): 2 ′-O,4 ′-C-methylene- linked bicyclic ribonucleotide
  modified_base (11)..(11): 5-methylcytosine; 2 ′-O,4 ′-C-methylene- linked bicyclic ribonucleotide
  modified_base (12)..(12): 2 ′-O,4 ′-C-methylene- linked bicyclic ribonucleotide
  modified_base (13)..(13): 2 ′-O,4 ′-C-methylene- linked bicyclic ribonucleotide
gtgcacttggcag

SEQ ID NO: 610 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): 5-methylcytosine; 2 ′-O,4 ′-C-methylene- linked bicyclic ribonucleotide
  modified_base (2)..(2): 5-methylcytosine; 2 ′-O,4 ′-C-methylene- linked bicyclic ribonucleotide
  modified_base (11)..(11): 5-methylcytosine; 2 ′-O,4 ′-C-methylene- linked bicyclic ribonucleotide
  modified_base (12)..(12): 2 ′-O,4 ′-C-methylene- linked bicyclic ribonucleotide
  modified_base (13)..(13): 2 ′-O,4 ′-C-methylene- linked bicyclic ribonucleotide
cctcagtgcactt

SEQ ID NO: 611 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): 2 ′-O,4 ′-C-methylene- linked bicyclic ribonucleotide
  modified_base (2)..(2): 5-methylcytosine; 2 ′-O,4 ′-C-methylene- linked bicyclic ribonucleotide
  modified_base (11)..(11): 2 ′-O,4 ′-C-methylene- linked bicyclic ribonucleotide
  modified_base (12)..(12): 5-methylcytosine; 2 ′-O,4 ′-C-methylene- linked bicyclic ribonucleotide
  modified_base (13)..(13): 2 ′-O,4 ′-C-methylene- linked bicyclic ribonucleotide
tcctcagtgcact

SEQ ID NO: 612 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): 5-methylcytosine; 2 ′-O,4 ′-C-methylene- linked bicyclic ribonucleotide
  modified_base (2)..(2): 5-methylcytosine; 2 ′-O,4 ′-C-methylene- linked bicyclic ribonucleotide
  modified_base (11)..(11): 2 ′-O,4 ′-C-methylene- linked bicyclic ribonucleotide
  modified_base (12)..(12): 2 ′-O,4 ′-C-methylene- linked bicyclic ribonucleotide
  modified_base (13)..(13): 5-methylcytosine
ccttcctcagtgc

SEQ ID NO: 613 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): 2 ′-O,4 ′-C-methylene- linked bicyclic ribonucleotide
  modified_base (2)..(2): 2 ′-O,4 ′-C-methylene- linked bicyclic ribonucleotide
  modified_base (11)..(11): 2 ′-O,4 ′-C-methylene- linked bicyclic ribonucleotide
  modified_base (12)..(12): 2 ′-O,4 ′-C-methylene- linked bicyclic ribonucleotide
  modified_base (13)..(13): 2 ′-O,4 ′-C-methylene- linked bicyclic ribonucleotide
agctgatggctta

SEQ ID NO: 614 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): 5-methylcytosine; 2 ′-O,4 ′-C-methylene- linked bicyclic ribonucleotide
  modified_base (2)..(2): 2 ′-O,4 ′-C-methylene- linked bicyclic ribonucleotide
  modified_base (11)..(11): 5-methylcytosine; 2 ′-O,4 ′-C-methylene- linked bicyclic ribonucleotide
  modified_base (12)..(12): 2 ′-O,4 ′-C-methylene- linked bicyclic ribonucleotide
  modified_base (13)..(13): 2 ′-O,4 ′-C-methylene- linked bicyclic ribonucleotide
cagctgatggctt

SEQ ID NO: 615 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): 5-methylcytosine; 2 ′-O,4 ′-C-methylene- linked bicyclic ribonucleotide
  modified_base (2)..(2): 5-methylcytosine; 2 ′-O,4 ′-C-methylene- linked bicyclic ribonucleotide
  modified_base (11)..(11): 2 ′-O,4 ′-C-methylene- linked bicyclic ribonucleotide
  modified_base (12)..(12): 5-methylcytosine; 2 ′-O,4 ′-C-methylene- linked bicyclic ribonucleotide
  modified_base (13)..(13): 2 ′-O,4 ′-C-methylene- linked bicyclic ribonucleotide
ccagctgatggct

SEQ ID NO: 616 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): 2 ′-O,4 ′-C-methylene- linked bicyclic ribonucleotide
  modified_base (2)..(2): 5-methylcytosine; 2 ′-O,4 ′-C-methylene- linked bicyclic ribonucleotide
  modified_base (11)..(11): 2 ′-O,4 ′-C-methylene- linked bicyclic ribonucleotide
  modified_base (12)..(12): 2 ′-O,4 ′-C-methylene- linked bicyclic ribonucleotide
  modified_base (13)..(13): 5-methylcytosine; 2 ′-O,4 ′-C-methylene- linked bicyclic ribonucleotide
tccagctgatggc

SEQ ID NO: 617 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): 2 ′-O,4 ′-C-methylene- linked bicyclic ribonucleotide
  modified_base (2)..(2): 2 ′-O,4 ′-C-methylene- linked bicyclic ribonucleotide
  modified_base (11)..(11): 2 ′-O,4 ′-C-methylene- linked bicyclic ribonucleotide
  modified_base (12)..(12): 2 ′-O,4 ′-C-methylene- linked bicyclic ribonucleotide
  modified_base (13)..(13): 2 ′-O,4 ′-C-methylene- linked bicyclic ribonucleotide
tgtttccagctga

SEQ ID NO: 618 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): 2 ′-O,4 ′-C-methylene- linked bicyclic ribonucleotide
  modified_base (2)..(2): 2 ′-O,4 ′-C-methylene- linked bicyclic ribonucleotide
  modified_base (11)..(11): 5-methylcytosine; 2 ′-O,4 ′-C-methylene- linked bicyclic ribonucleotide
  modified_base (12)..(12): 2 ′-O,4 ′-C-methylene- linked bicyclic ribonucleotide
  modified_base (13)..(13): 2 ′-O,4 ′-C-methylene- linked bicyclic ribonucleotide
ttgtttccagctg

SEQ ID NO: 619 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): 2 ′-O,4 ′-C-methylene- linked bicyclic ribonucleotide
  modified_base (2)..(2): 5-methylcytosine; 2 ′-O,4 ′-C-methylene- linked bicyclic ribonucleotide
  modified_base (11)..(11): 2 ′-O,4 ′-C-methylene- linked bicyclic ribonucleotide
  modified_base (12)..(12): 5-methylcytosine; 2 ′-O,4 ′-C-methylene- linked bicyclic ribonucleotide
  modified_base (13)..(13): 2 ′-O,4 ′-C-methylene- linked bicyclic ribonucleotide
gcatgttgtggca

SEQ ID NO: 620 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): 5-methylcytosine; 2 ′-O,4 ′-C-methylene- linked bicyclic ribonucleotide
  modified_base (2)..(2): 2 ′-O,4 ′-C-methylene- linked bicyclic ribonucleotide
  modified_base (11)..(11): 2 ′-O,4 ′-C-methylene- linked bicyclic ribonucleotide
  modified_base (12)..(12): 2 ′-O,4 ′-C-methylene- linked bicyclic ribonucleotide
  modified_base (13)..(13): 5-methylcytosine; 2 ′-O,4 ′-C-methylene- linked bicyclic ribonucleotide
cgcatgttgtggc

SEQ ID NO: 621 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): 5-methylcytosine; 2 ′-O,4 ′-C-methylene- linked bicyclic ribonucleotide
  modified_base (2)..(2): 2 ′-O,4 ′-C-methylene- linked bicyclic ribonucleotide
  modified_base (11)..(11): 2 ′-O,4 ′-C-methylene- linked bicyclic ribonucleotide
  modified_base (12)..(12): 5-methylcytosine; 2 ′-O,4 ′-C-methylene- linked bicyclic ribonucleotide
  modified_base (13)..(13): 2 ′-O,4 ′-C-methylene- linked bicyclic ribonucleotide
cattgtctggtct

SEQ ID NO: 622 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): 5-methylcytosine; 2 ′-O,4 ′-C-methylene- linked bicyclic ribonucleotide
  modified_base (2)..(2): 5-methylcytosine; 2 ′-O,4 ′-C-methylene- linked bicyclic ribonucleotide
  modified_base (11)..(11): 2 ′-O,4 ′-C-methylene- linked bicyclic ribonucleotide
  modified_base (12)..(12): 2 ′-O,4 ′-C-methylene- linked bicyclic ribonucleotide
  modified_base (13)..(13): 5-methylcytosine; 2 ′-O,4 ′-C-methylene- linked bicyclic ribonucleotide
ccattgtctggtc

SEQ ID NO: 623 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): 2 ′-O,4 ′-C-methylene- linked bicyclic ribonucleotide
  modified_base (2)..(2): 5-methylcytosine; 2 ′-O,4 ′-C-methylene- linked bicyclic ribonucleotide
  modified_base (11)..(11): 2 ′-O,4 ′-C-methylene- linked bicyclic ribonucleotide
  modified_base (12)..(12): 2 ′-O,4 ′-C-methylene- linked bicyclic ribonucleotide
  modified_base (13)..(13): 2 ′-O,4 ′-C-methylene- linked bicyclic ribonucleotide
tccattgtctggt

SEQ ID NO: 624 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): 2 ′-O,4 ′-C-methylene- linked bicyclic ribonucleotide
  modified_base (2)..(2): 2 ′-O,4 ′-C-methylene- linked bicyclic ribonucleotide
  modified_base (11)..(11): 2 ′-O,4 ′-C-methylene- linked bicyclic ribonucleotide
  modified_base (12)..(12): 2 ′-O,4 ′-C-methylene- linked bicyclic ribonucleotide
  modified_base (13)..(13): 2 ′-O,4 ′-C-methylene- linked bicyclic ribonucleotide
atccattgtctgg

SEQ ID NO: 625 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): 2 ′-O,4 ′-C-methylene- linked bicyclic ribonucleotide
  modified_base (2)..(2): 2 ′-O,4 ′-C-methylene- linked bicyclic ribonucleotide
  modified_base (11)..(11): 2 ′-O,4 ′-C-methylene- linked bicyclic ribonucleotide
  modified_base (12)..(12): 2 ′-O,4 ′-C-methylene- linked bicyclic ribonucleotide
  modified_base (13)..(13): 5-methylcytosine; 2 ′-O,4 ′-C-methylene- linked bicyclic ribonucleotide
atgtggctttatc

SEQ ID NO: 626 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): 2 ′-O,4 ′-C-methylene- linked bicyclic ribonucleotide
  modified_base (2)..(2): 2 ′-O,4 ′-C-methylene- linked bicyclic ribonucleotide
  modified_base (11)..(11): 2 ′-O,4 ′-C-methylene- linked bicyclic ribonucleotide
  modified_base (12)..(12): 2 ′-O,4 ′-C-methylene- linked bicyclic ribonucleotide
  modified_base (13)..(13): 2 ′-O,4 ′-C-methylene- linked bicyclic ribonucleotide
aatgtggctttat

SEQ ID NO: 627 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): 5-methylcytosine; 2 ′-O,4 ′-C-methylene- linked bicyclic ribonucleotide
  modified_base (2)..(2): 5-methylcytosine; 2 ′-O,4 ′-C-methylene- linked bicyclic ribonucleotide
  modified_base (11)..(11): 5-methylcytosine; 2 ′-O,4 ′-C-methylene- linked bicyclic ribonucleotide
  modified_base (12)..(12): 2 ′-O,4 ′-C-methylene- linked bicyclic ribonucleotide
  modified_base (13)..(13): 2 ′-O,4 ′-C-methylene- linked bicyclic ribonucleotide
ccaaatgtggctt

SEQ ID NO: 628 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): 5-methylcytosine; 2 ′-O,4 ′-C-methylene- linked bicyclic ribonucleotide
  modified_base (2)..(2): 5-methylcytosine; 2 ′-O,4 ′-C-methylene- linked bicyclic ribonucleotide
  modified_base (11)..(11): 2 ′-O,4 ′-C-methylene- linked bicyclic ribonucleotide
  modified_base (12)..(12): 2 ′-O,4 ′-C-methylene- linked bicyclic ribonucleotide
  modified_base (13)..(13): 5-methylcytosine; 2 ′-O,4 ′-C-methylene- linked bicyclic ribonucleotide
ccatcatggcagc

SEQ ID NO: 629 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): 2 ′-O,4 ′-C-methylene- linked bicyclic ribonucleotide
  modified_base (2)..(2): 2 ′-O,4 ′-C-methylene- linked bicyclic ribonucleotide
  modified_base (11)..(11): 2 ′-O,4 ′-C-methylene- linked bicyclic ribonucleotide
  modified_base (12)..(12): 2 ′-O,4 ′-C-methylene- linked bicyclic ribonucleotide
  modified_base (13)..(13): 5-methylcytosine; 2 ′-O,4 ′-C-methylene- linked bicyclic ribonucleotide
tatccatcatggc

SEQ ID NO: 630 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): 5-methylcytosine; 2 ′-O,4 ′-C-methylene- linked bicyclic ribonucleotide
  modified_base (2)..(2): 5-methylcytosine; 2 ′-O,4 ′-C-methylene- linked bicyclic ribonucleotide
  modified_base (11)..(11): 2 ′-O,4 ′-C-methylene- linked bicyclic ribonucleotide
  modified_base (12)..(12): 2 ′-O,4 ′-C-methylene- linked bicyclic ribonucleotide
  modified_base (13)..(13): 5-methylcytosine; 2 ′-O,4 ′-C-methylene- linked bicyclic ribonucleotide
cccagtttggtac

SEQ ID NO: 631 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): 2 ′-O,4 ′-C-methylene- linked bicyclic ribonucleotide
  modified_base (2)..(2): 2 ′-O,4 ′-C-methylene- linked bicyclic ribonucleotide
  modified_base (11)..(11): 5-methylcytosine; 2 ′-O,4 ′-C-methylene- linked bicyclic ribonucleotide
  modified_base (12)..(12): 5-methylcytosine; 2 ′-O,4 ′-C-methylene- linked bicyclic ribonucleotide
  modified_base (13)..(13): 2 ′-O,4 ′-C-methylene- linked bicyclic ribonucleotide
gaccagtttccca

SEQ ID NO: 632 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): 2 ′-O,4 ′-C-methylene- linked bicyclic ribonucleotide
  modified_base (2)..(2): 2 ′-O,4 ′-C-methylene- linked bicyclic ribonucleotide
  modified_base (11)..(11): 5-methylcytosine; 2 ′-O,4 ′-C-methylene- linked bicyclic ribonucleotide
  modified_base (12)..(12): 5-methylcytosine; 2 ′-O,4 ′-C-methylene- linked bicyclic ribonucleotide
  modified_base (13)..(13): 5-methylcytosine; 2 ′-O,4 ′-C-methylene- linked bicyclic ribonucleotide
agaccagtttccc

SEQ ID NO: 633 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): 2 ′-O,4 ′-C-methylene- linked bicyclic ribonucleotide
  modified_base (2)..(2): 2 ′-O,4 ′-C-methylene- linked bicyclic ribonucleotide
  modified_base (11)..(11): 2 ′-O,4 ′-C-methylene- linked bicyclic ribonucleotide
  modified_base (12)..(12): 2 ′-O,4 ′-C-methylene- linked bicyclic ribonucleotide
  modified_base (13)..(13): 2 ′-O,4 ′-C-methylene- linked bicyclic ribonucleotide
ggcagaccagttt

SEQ ID NO: 634 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): 2 ′-O,4 ′-C-methylene- linked bicyclic ribonucleotide
  modified_base (2)..(2): 2 ′-O,4 ′-C-methylene- linked bicyclic ribonucleotide
  modified_base (11)..(11): 2 ′-O,4 ′-C-methylene- linked bicyclic ribonucleotide
  modified_base (12)..(12): 2 ′-O,4 ′-C-methylene- linked bicyclic ribonucleotide
  modified_base (13)..(13): 2 ′-O,4 ′-C-methylene- linked bicyclic ribonucleotide
tggcagaccagtt

SEQ ID NO: 635 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): 5-methylcytosine; 2 ′-O,4 ′-C-methylene- linked bicyclic ribonucleotide
  modified_base (2)..(2): 2 ′-O,4 ′-C-methylene- linked bicyclic ribonucleotide
  modified_base (11)..(11): 5-methylcytosine; 2 ′-O,4 ′-C-methylene- linked bicyclic ribonucleotide
  modified_base (12)..(12): 2 ′-O,4 ′-C-methylene- linked bicyclic ribonucleotide
  modified_base (13)..(13): 2 ′-O,4 ′-C-methylene- linked bicyclic ribonucleotide
catggcagaccag

SEQ ID NO: 636 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): 2 ′-O,4 ′-C-methylene- linked bicyclic ribonucleotide
  modified_base (2)..(2): 5-methylcytosine; 2 ′-O,4 ′-C-methylene- linked bicyclic ribonucleotide
  modified_base (11)..(11): 5-methylcytosine; 2 ′-O,4 ′-C-methylene- linked bicyclic ribonucleotide
  modified_base (12)..(12): 5-methylcytosine; 2 ′-O,4 ′-C-methylene- linked bicyclic ribonucleotide
  modified_base (13)..(13): 2 ′-O,4 ′-C-methylene- linked bicyclic ribonucleotide
gcatggcagacca

SEQ ID NO: 637 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): 2 ′-O,4 ′-C-methylene- linked bicyclic ribonucleotide
  modified_base (2)..(2): 2 ′-O,4 ′-C-methylene- linked bicyclic ribonucleotide
  modified_base (11)..(11): 2 ′-O,4 ′-C-methylene- linked bicyclic ribonucleotide
  modified_base (12)..(12): 5-methylcytosine; 2 ′-O,4 ′-C-methylene- linked bicyclic ribonucleotide
  modified_base (13)..(13): 5-methylcytosine; 2 ′-O,4 ′-C-methylene- linked bicyclic ribonucleotide
ggcatggcagacc

SEQ ID NO: 638 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): 2 ′-O,4 ′-C-methylene- linked bicyclic ribonucleotide
  modified_base (2)..(2): 2 ′-O,4 ′-C-methylene- linked bicyclic ribonucleotide
  modified_base (11)..(11): 2 ′-O,4 ′-C-methylene- linked bicyclic ribonucleotide
  modified_base (12)..(12): 2 ′-O,4 ′-C-methylene- linked bicyclic ribonucleotide
  modified_base (13)..(13): 5-methylcytosine; 2 ′-O,4 ′-C-methylene- linked bicyclic ribonucleotide
tggcatggcagac

SEQ ID NO: 639 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): 5-methylcytosine; 2 ′-O,4 ′-C-methylene- linked bicyclic ribonucleotide
  modified_base (2)..(2): 2 ′-O,4 ′-C-methylene- linked bicyclic ribonucleotide
  modified_base (11)..(11): 5-methylcytosine; 2 ′-O,4 ′-C-methylene- linked bicyclic ribonucleotide
  modified_base (12)..(12): 2 ′-O,4 ′-C-methylene- linked bicyclic ribonucleotide
  modified_base (13)..(13): 2 ′-O,4 ′-C-methylene- linked bicyclic ribonucleotide
cttggcatggcag

SEQ ID NO: 640 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): 2 ′-O,4 ′-C-methylene- linked bicyclic ribonucleotide
  modified_base (2)..(2): 5-methylcytosine; 2 ′-O,4 ′-C-methylene- linked bicyclic ribonucleotide
  modified_base (11)..(11): 2 ′-O,4 ′-C-methylene- linked bicyclic ribonucleotide
  modified_base (12)..(12): 5-methylcytosine; 2 ′-O,4 ′-C-methylene- linked bicyclic ribonucleotide
  modified_base (13)..(13): 2 ′-O,4 ′-C-methylene- linked bicyclic ribonucleotide
acttggcatggca

SEQ ID NO: 641 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): 2 ′-O,4 ′-C-methylene- linked bicyclic ribonucleotide
  modified_base (2)..(2): 2 ′-O,4 ′-C-methylene- linked bicyclic ribonucleotide
  modified_base (11)..(11): 2 ′-O,4 ′-C-methylene- linked bicyclic ribonucleotide
  modified_base (12)..(12): 2 ′-O,4 ′-C-methylene- linked bicyclic ribonucleotide
  modified_base (13)..(13): 5-methylcytosine; 2 ′-O,4 ′-C-methylene- linked bicyclic ribonucleotide
aacttggcatggc

SEQ ID NO: 642 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): 2 ′-O,4 ′-C-methylene- linked bicyclic ribonucleotide
  modified_base (2)..(2): 5-methylcytosine; 2 ′-O,4 ′-C-methylene- linked bicyclic ribonucleotide
  modified_base (11)..(11): 2 ′-O,4 ′-C-methylene- linked bicyclic ribonucleotide
  modified_base (12)..(12): 2 ′-O,4 ′-C-methylene- linked bicyclic ribonucleotide
  modified_base (13)..(13): 5-methylcytosine; 2 ′-O,4 ′-C-methylene- linked bicyclic ribonucleotide
acaccacagtggc

SEQ ID NO: 643 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): 2 ′-O,4 ′-C-methylene- linked bicyclic ribonucleotide
  modified_base (2)..(2): 2 ′-O,4 ′-C-methylene- linked bicyclic ribonucleotide
  modified_base (11)..(11): 2 ′-O,4 ′-C-methylene- linked bicyclic ribonucleotide
  modified_base (12)..(12): 2 ′-O,4 ′-C-methylene- linked bicyclic ribonucleotide
  modified_base (13)..(13): 2 ′-O,4 ′-C-methylene- linked bicyclic ribonucleotide
ggtacaccacagt

SEQ ID NO: 644 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): 5-methylcytosine; 2 ′-O,4 ′-C-methylene- linked bicyclic ribonucleotide
  modified_base (2)..(2): 5-methylcytosine; 2 ′-O,4 ′-C-methylene- linked bicyclic ribonucleotide
  modified_base (11)..(11): 5-methylcytosine; 2 ′-O,4 ′-C-methylene- linked bicyclic ribonucleotide
  modified_base (12)..(12): 5-methylcytosine; 2 ′-O,4 ′-C-methylene- linked bicyclic ribonucleotide
  modified_base (13)..(13): 2 ′-O,4 ′-C-methylene- linked bicyclic ribonucleotide
ccttggtacacca

SEQ ID NO: 645 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): 2 ′-O,4 ′-C-methylene- linked bicyclic ribonucleotide
  modified_base (2)..(2): 5-methylcytosine; 2 ′-O,4 ′-C-methylene- linked bicyclic ribonucleotide
  modified_base (11)..(11): 2 ′-O,4 ′-C-methylene- linked bicyclic ribonucleotide
  modified_base (12)..(12): 5-methylcytosine; 2 ′-O,4 ′-C-methylene- linked bicyclic ribonucleotide
  modified_base (13)..(13): 5-methylcytosine; 2 ′-O,4 ′-C-methylene- linked bicyclic ribonucleotide
tccttggtacacc

SEQ ID NO: 646 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): 5-methylcytosine; 2 ′-O,4 ′-C-methylene- linked bicyclic ribonucleotide
  modified_base (2)..(2): 2 ′-O,4 ′-C-methylene- linked bicyclic ribonucleotide
  modified_base (11)..(11): 5-methylcytosine; 2 ′-O,4 ′-C-methylene- linked bicyclic ribonucleotide
  modified_base (12)..(12): 2 ′-O,4 ′-C-methylene- linked bicyclic ribonucleotide
  modified_base (13)..(13): 5-methylcytosine; 2 ′-O,4 ′-C-methylene- linked bicyclic ribonucleotide
ctccttggtacac

SEQ ID NO: 647 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): 5-methylcytosine; 2 ′-O,4 ′-C-methylene- linked bicyclic ribonucleotide
  modified_base (2)..(2): 2 ′-O,4 ′-C-methylene- linked bicyclic ribonucleotide
  modified_base (11)..(11): 2 ′-O,4 ′-C-methylene- linked bicyclic ribonucleotide
  modified_base (12)..(12): 2 ′-O,4 ′-C-methylene- linked bicyclic ribonucleotide
  modified_base (13)..(13): 5-methylcytosine; 2 ′-O,4 ′-C-methylene- linked bicyclic ribonucleotide
ctctccttggtac

SEQ ID NO: 648 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): 5-methylcytosine; 2 ′-O,4 ′-C-methylene- linked bicyclic ribonucleotide
  modified_base (2)..(2): 2 ′-O,4 ′-C-methylene- linked bicyclic ribonucleotide
  modified_base (11)..(11): 2 ′-O,4 ′-C-methylene- linked bicyclic ribonucleotide
  modified_base (12)..(12): 2 ′-O,4 ′-C-methylene- linked bicyclic ribonucleotide
  modified_base (13)..(13): 2 ′-O,4 ′-C-methylene- linked bicyclic ribonucleotide
ctctctccttggt

SEQ ID NO: 649 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): 2 ′-O,4 ′-C-methylene- linked bicyclic ribonucleotide
  modified_base (2)..(2): 5-methylcytosine; 2 ′-O,4 ′-C-methylene- linked bicyclic ribonucleotide
  modified_base (11)..(11): 2 ′-O,4 ′-C-methylene- linked bicyclic ribonucleotide
  modified_base (12)..(12): 2 ′-O,4 ′-C-methylene- linked bicyclic ribonucleotide
  modified_base (13)..(13): 2 ′-O,4 ′-C-methylene- linked bicyclic ribonucleotide
tctctctccttgg

SEQ ID NO: 650 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): 5-methylcytosine; 2 ′-O,4 ′-C-methylene- linked bicyclic ribonucleotide
  modified_base (2)..(2): 2 ′-O,4 ′-C-methylene- linked bicyclic ribonucleotide
  modified_base (11)..(11): 2 ′-O,4 ′-C-methylene- linked bicyclic ribonucleotide
  modified_base (12)..(12): 2 ′-O,4 ′-C-methylene- linked bicyclic ribonucleotide
  modified_base (13)..(13): 2 ′-O,4 ′-C-methylene- linked bicyclic ribonucleotide
ctctctctccttg

SEQ ID NO: 651 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): 2 ′-O,4 ′-C-methylene- linked bicyclic ribonucleotide
  modified_base (2)..(2): 2 ′-O,4 ′-C-methylene- linked bicyclic ribonucleotide
  modified_base (11)..(11): 2 ′-O,4 ′-C-methylene- linked bicyclic ribonucleotide
  modified_base (12)..(12): 5-methylcytosine; 2 ′-O,4 ′-C-methylene- linked bicyclic ribonucleotide
  modified_base (13)..(13): 2 ′-O,4 ′-C-methylene- linked bicyclic ribonucleotide
agcattccattct

SEQ ID NO: 652 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): 2 ′-O,4 ′-C-methylene- linked bicyclic ribonucleotide
  modified_base (2)..(2): 2 ′-O,4 ′-C-methylene- linked bicyclic ribonucleotide
  modified_base (11)..(11): 2 ′-O,4 ′-C-methylene- linked bicyclic ribonucleotide
  modified_base (12)..(12): 5-methylcytosine; 2 ′-O,4 ′-C-methylene- linked bicyclic ribonucleotide
  modified_base (13)..(13): 2 ′-O,4 ′-C-methylene- linked bicyclic ribonucleotide
gttccttgaagca

SEQ ID NO: 653 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): 2 ′-O,4 ′-C-methylene- linked bicyclic ribonucleotide
  modified_base (2)..(2): 2 ′-O,4 ′-C-methylene- linked bicyclic ribonucleotide
  modified_base (11)..(11): 2 ′-O,4 ′-C-methylene- linked bicyclic ribonucleotide
  modified_base (12)..(12): 2 ′-O,4 ′-C-methylene- linked bicyclic ribonucleotide
  modified_base (13)..(13): 5-methylcytosine; 2 ′-O,4 ′-C-methylene- linked bicyclic ribonucleotide
tgttccttgaagc

SEQ ID NO: 654 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): 2 ′-O,4 ′-C-methylene- linked bicyclic ribonucleotide
  modified_base (2)..(2): 2 ′-O,4 ′-C-methylene- linked bicyclic ribonucleotide
  modified_base (11)..(11): 2 ′-O,4 ′-C-methylene- linked bicyclic ribonucleotide
  modified_base (12)..(12): 2 ′-O,4 ′-C-methylene- linked bicyclic ribonucleotide
  modified_base (13)..(13): 2 ′-O,4 ′-C-methylene- linked bicyclic ribonucleotide
tgtgttccttgaa

SEQ ID NO: 655 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): 5-methylcytosine; 2 ′-O,4 ′-C-methylene- linked bicyclic ribonucleotide
  modified_base (2)..(2): 2 ′-O,4 ′-C-methylene- linked bicyclic ribonucleotide
  modified_base (11)..(11): 2 ′-O,4 ′-C-methylene- linked bicyclic ribonucleotide
  modified_base (12)..(12): 2 ′-O,4 ′-C-methylene- linked bicyclic ribonucleotide
  modified_base (13)..(13): 2 ′-O,4 ′-C-methylene- linked bicyclic ribonucleotide
ctgtgttccttga

SEQ ID NO: 656 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): 2 ′-O,4 ′-C-methylene- linked bicyclic ribonucleotide
  modified_base (2)..(2): 5-methylcytosine; 2 ′-O,4 ′-C-methylene- linked bicyclic ribonucleotide
  modified_base (11)..(11): 2 ′-O,4 ′-C-methylene- linked bicyclic ribonucleotide
  modified_base (12)..(12): 2 ′-O,4 ′-C-methylene- linked bicyclic ribonucleotide
  modified_base (13)..(13): 2 ′-O,4 ′-C-methylene- linked bicyclic ribonucleotide
actgtgttccttg

SEQ ID NO: 657 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): 2 ′-O,4 ′-C-methylene- linked bicyclic ribonucleotide
  modified_base (2)..(2): 2 ′-O,4 ′-C-methylene- linked bicyclic ribonucleotide
  modified_base (11)..(11): 5-methylcytosine; 2 ′-O,4 ′-C-methylene- linked bicyclic ribonucleotide
  modified_base (12)..(12): 5-methylcytosine; 2 ′-O,4 ′-C-methylene- linked bicyclic ribonucleotide
  modified_base (13)..(13): 2 ′-O,4 ′-C-methylene- linked bicyclic ribonucleotide
gaactgtgttcct

SEQ ID NO: 658 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): 2 ′-O,4 ′-C-methylene- linked bicyclic ribonucleotide
  modified_base (2)..(2): 2 ′-O,4 ′-C-methylene- linked bicyclic ribonucleotide
  modified_base (11)..(11): 2 ′-O,4 ′-C-methylene- linked bicyclic ribonucleotide
  modified_base (12)..(12): 5-methylcytosine; 2 ′-O,4 ′-C-methylene- linked bicyclic ribonucleotide
  modified_base (13)..(13): 5-methylcytosine; 2 ′-O,4 ′-C-methylene- linked bicyclic ribonucleotide
agaactgtgttcc

SEQ ID NO: 659 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): 2 ′-O,4 ′-C-methylene- linked bicyclic ribonucleotide
  modified_base (2)..(2): 5-methylcytosine; 2 ′-O,4 ′-C-methylene- linked bicyclic ribonucleotide
  modified_base (11)..(11): 2 ′-O,4 ′-C-methylene- linked bicyclic ribonucleotide
  modified_base (12)..(12): 2 ′-O,4 ′-C-methylene- linked bicyclic ribonucleotide
  modified_base (13)..(13): 2 ′-O,4 ′-C-methylene- linked bicyclic ribonucleotide
gccagagaactgt

SEQ ID NO: 660 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): 2 ′-O,4 ′-C-methylene- linked bicyclic ribonucleotide
  modified_base (2)..(2): 2 ′-O,4 ′-C-methylene- linked bicyclic ribonucleotide
  modified_base (11)..(11): 5-methylcytosine; 2 ′-O,4 ′-C-methylene- linked bicyclic ribonucleotide
  modified_base (12)..(12): 2 ′-O,4 ′-C-methylene- linked bicyclic ribonucleotide
  modified_base (13)..(13): 2 ′-O,4 ′-C-methylene- linked bicyclic ribonucleotide
agccagagaactg

SEQ ID NO: 661 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): 2 ′-O,4 ′-C-methylene- linked bicyclic ribonucleotide
  modified_base (2)..(2): 2 ′-O,4 ′-C-methylene- linked bicyclic ribonucleotide
  modified_base (11)..(11): 2 ′-O,4 ′-C-methylene- linked bicyclic ribonucleotide
  modified_base (12)..(12): 2 ′-O,4 ′-C-methylene- linked bicyclic ribonucleotide
  modified_base (13)..(13): 2 ′-O,4 ′-C-methylene- linked bicyclic ribonucleotide
gatgcatcagttt

SEQ ID NO: 662 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): 2 ′-O,4 ′-C-methylene- linked bicyclic ribonucleotide
  modified_base (2)..(2): 2 ′-O,4 ′-C-methylene- linked bicyclic ribonucleotide
  modified_base (11)..(11): 2 ′-O,4 ′-C-methylene- linked bicyclic ribonucleotide
  modified_base (12)..(12): 2 ′-O,4 ′-C-methylene- linked bicyclic ribonucleotide
  modified_base (13)..(13): 2 ′-O,4 ′-C-methylene- linked bicyclic ribonucleotide
ggatgcatcagtt

SEQ ID NO: 663 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): 5-methylcytosine; 2 ′-O,4 ′-C-methylene- linked bicyclic ribonucleotide
  modified_base (2)..(2): 2 ′-O,4 ′-C-methylene- linked bicyclic ribonucleotide
  modified_base (11)..(11): 2 ′-O,4 ′-C-methylene- linked bicyclic ribonucleotide
  modified_base (12)..(12): 2 ′-O,4 ′-C-methylene- linked bicyclic ribonucleotide
  modified_base (13)..(13): 2 ′-O,4 ′-C-methylene- linked bicyclic ribonucleotide
cggatgcatcagt

SEQ ID NO: 664 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): 2 ′-O,4 ′-C-methylene- linked bicyclic ribonucleotide
  modified_base (2)..(2): 5-methylcytosine; 2 ′-O,4 ′-C-methylene- linked bicyclic ribonucleotide
  modified_base (11)..(11): 5-methylcytosine; 2 ′-O,4 ′-C-methylene- linked bicyclic ribonucleotide
  modified_base (12)..(12): 2 ′-O,4 ′-C-methylene- linked bicyclic ribonucleotide
  modified_base (13)..(13): 2 ′-O,4 ′-C-methylene- linked bicyclic ribonucleotide
tcggatgcatcag

SEQ ID NO: 665 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): 2 ′-O,4 ′-C-methylene- linked bicyclic ribonucleotide
  modified_base (2)..(2): 2 ′-O,4 ′-C-methylene- linked bicyclic ribonucleotide
  modified_base (11)..(11): 2 ′-O,4 ′-C-methylene- linked bicyclic ribonucleotide
  modified_base (12)..(12): 5-methylcytosine; 2 ′-O,4 ′-C-methylene- linked bicyclic ribonucleotide
  modified_base (13)..(13): 2 ′-O,4 ′-C-methylene- linked bicyclic ribonucleotide
atcggatgcatca

SEQ ID NO: 666 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): 2 ′-O,4 ′-C-methylene- linked bicyclic ribonucleotide
  modified_base (2)..(2): 2 ′-O,4 ′-C-methylene- linked bicyclic ribonucleotide
  modified_base (11)..(11): 5-methylcytosine; 2 ′-O,4 ′-C-methylene- linked bicyclic ribonucleotide
  modified_base (12)..(12): 2 ′-O,4 ′-C-methylene- linked bicyclic ribonucleotide
  modified_base (13)..(13): 2 ′-O,4 ′-C-methylene- linked bicyclic ribonucleotide
ggctttacatcgg

SEQ ID NO: 667 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): 2 ′-O,4 ′-C-methylene- linked bicyclic ribonucleotide
  modified_base (2)..(2): 2 ′-O,4 ′-C-methylene- linked bicyclic ribonucleotide
  modified_base (11)..(11): 2 ′-O,4 ′-C-methylene- linked bicyclic ribonucleotide
  modified_base (12)..(12): 2 ′-O,4 ′-C-methylene- linked bicyclic ribonucleotide
  modified_base (13)..(13): 2 ′-O,4 ′-C-methylene- linked bicyclic ribonucleotide
agtgtgacatttt

SEQ ID NO: 668 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): 2 ′-O,4 ′-C-methylene- linked bicyclic ribonucleotide
  modified_base (2)..(2): 2 ′-O,4 ′-C-methylene- linked bicyclic ribonucleotide
  modified_base (11)..(11): 2 ′-O,4 ′-C-methylene- linked bicyclic ribonucleotide
  modified_base (12)..(12): 2 ′-O,4 ′-C-methylene- linked bicyclic ribonucleotide
  modified_base (13)..(13): 2 ′-O,4 ′-C-methylene- linked bicyclic ribonucleotide
aagtgtgacattt

SEQ ID NO: 669 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): 2 ′-O,4 ′-C-methylene- linked bicyclic ribonucleotide
  modified_base (2)..(2): 2 ′-O,4 ′-C-methylene- linked bicyclic ribonucleotide
  modified_base (11)..(11): 2 ′-O,4 ′-C-methylene- linked bicyclic ribonucleotide
  modified_base (12)..(12): 2 ′-O,4 ′-C-methylene- linked bicyclic ribonucleotide
  modified_base (13)..(13): 2 ′-O,4 ′-C-methylene- linked bicyclic ribonucleotide
ggttccttggatg

SEQ ID NO: 670 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): 2 ′-O,4 ′-C-methylene- linked bicyclic ribonucleotide
  modified_base (2)..(2): 2 ′-O,4 ′-C-methylene- linked bicyclic ribonucleotide
  modified_base (11)..(11): 2 ′-O,4 ′-C-methylene- linked bicyclic ribonucleotide
  modified_base (12)..(12): 2 ′-O,4 ′-C-methylene- linked bicyclic ribonucleotide
  modified_base (13)..(13): 2 ′-O,4 ′-C-methylene- linked bicyclic ribonucleotide
aggttccttggat

SEQ ID NO: 671 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): 2 ′-O,4 ′-C-methylene- linked bicyclic ribonucleotide
  modified_base (2)..(2): 2 ′-O,4 ′-C-methylene- linked bicyclic ribonucleotide
  modified_base (11)..(11): 2 ′-O,4 ′-C-methylene- linked bicyclic ribonucleotide
  modified_base (12)..(12): 2 ′-O,4 ′-C-methylene- linked bicyclic ribonucleotide
  modified_base (13)..(13): 2 ′-O,4 ′-C-methylene- linked bicyclic ribonucleotide
taggttccttgga

SEQ ID NO: 672 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): 2 ′-O,4 ′-C-methylene- linked bicyclic ribonucleotide
  modified_base (2)..(2): 2 ′-O,4 ′-C-methylene- linked bicyclic ribonucleotide
  modified_base (11)..(11): 2 ′-O,4 ′-C-methylene- linked bicyclic ribonucleotide
  modified_base (12)..(12): 2 ′-O,4 ′-C-methylene- linked bicyclic ribonucleotide
  modified_base (13)..(13): 2 ′-O,4 ′-C-methylene- linked bicyclic ribonucleotide
ttaggttccttgg

SEQ ID NO: 673 - DNA, 13 bp, Artificial Sequence
  Synthetic oligonucleotideCombined DNA/RNA Molecule nucleotides 3 to 10 are deoxyribonucleotides-other nucleotides are ribonucleotides
  modified_base (1)..(1): 2 ′-O,4 ′-C-methylene- linked bicyclic ribonucleotide
  modified_base (2)..(2): 2 ′-O,4 ′-C-methylene- linked bicyclic ribonucleotide
  modified_base (11)..(11): 2 ′-O,4 ′-C-methylene- linked bicyclic ribonucleotide
  modified_base (12)..(12): 2 ′-O,4 ′-C-methylene- linked bicyclic ribonucleotide
  modified_base (13)..(13): 2 ′-O,4 ′-C-methylene- linked bicyclic ribonucleotide
ggacatactctgg

SEQ ID NO: 674 - DNA, 13 bp, Artificial Sequence
  Synthetic oligonucleotideCombined DNA/RNA Molecule nucleotides 4 to 10 are deoxyribonucleotides-other nucleotides are ribonucleotides
  modified_base (1)..(1): 2 ′-O,4 ′-C-methylene- linked bicyclic ribonucleotide
  modified_base (2)..(2): 2 ′-O,4 ′-C-methylene- linked bicyclic ribonucleotide
  modified_base (3)..(3): 2 ′-O,4 ′-C-methylene- linked bicyclic ribonucleotide
  modified_base (11)..(11): 2 ′-O,4 ′-C-methylene- linked bicyclic ribonucleotide
  modified_base (12)..(12): 2 ′-O,4 ′-C-methylene- linked bicyclic ribonucleotide
  modified_base (13)..(13): 2 ′-O,4 ′-C-methylene- linked bicyclic ribonucleotide
ggacatactctgg

SEQ ID NO: 675 - DNA, 14 bp, Artificial Sequence
  Synthetic oligonucleotideCombined DNA/RNA Molecule nucleotides 4 to 11 are deoxyribonucleotides-other nucleotides are ribonucleotides
  modified_base (1)..(1): 2 ′-O,4 ′-C-methylene- linked bicyclic ribonucleotide
  modified_base (2)..(2): 2 ′-O,4 ′-C-methylene- linked bicyclic ribonucleotide
  modified_base (3)..(3): 2 ′-O,4 ′-C-methylene- linked bicyclic ribonucleotide
  modified_base (12)..(12): 2 ′-O,4 ′-C-methylene- linked bicyclic ribonucleotide
  modified_base (13)..(13): 2 ′-O,4 ′-C-methylene- linked bicyclic ribonucleotide
  modified_base (14)..(14): 2 ′-O,4 ′-C-methylene- linked bicyclic ribonucleotide
aggacatactctgg

SEQ ID NO: 676 - DNA, 15 bp, Artificial Sequence
  Synthetic oligonucleotideCombined DNA/RNA Molecule nucleotides 4 to 12 are deoxyribonucleotides-other nucleotides are ribonucleotides
  modified_base (1)..(1): 2 ′-O,4 ′-C-methylene- linked bicyclic ribonucleotide
  modified_base (2)..(2): 2 ′-O,4 ′-C-methylene- linked bicyclic ribonucleotide
  modified_base (3)..(3): 2 ′-O,4 ′-C-methylene- linked bicyclic ribonucleotide
  modified_base (13)..(13): 2 ′-O,4 ′-C-methylene- linked bicyclic ribonucleotide
  modified_base (14)..(14): 2 ′-O,4 ′-C-methylene- linked bicyclic ribonucleotide
  modified_base (15)..(15): 2 ′-O,4 ′-C-methylene- linked bicyclic ribonucleotide
aaggacatactctgg

SEQ ID NO: 677 - DNA, 15 bp, Artificial Sequence
  Synthetic oligonucleotideCombined DNA/RNA Molecule nucleotides 4 to 11 are deoxyribonucleotides-other nucleotides are ribonucleotides
  modified_base (1)..(1): 2 ′-O,4 ′-C-methylene- linked bicyclic ribonucleotide
  modified_base (2)..(2): 2 ′-O,4 ′-C-methylene- linked bicyclic ribonucleotide
  modified_base (3)..(3): 2 ′-O,4 ′-C-methylene- linked bicyclic ribonucleotide
  modified_base (12)..(12): 5-methylcytosine; 2 ′-O,4 ′-C-methylene- linked bicyclic ribonucleotide
  modified_base (13)..(13): 2 ′-O,4 ′-C-methylene- linked bicyclic ribonucleotide
  modified_base (14)..(14): 2 ′-O,4 ′-C-methylene- linked bicyclic ribonucleotide
  modified_base (15)..(15): 2 ′-O,4 ′-C-methylene- linked bicyclic ribonucleotide
aaggacatactctgg

SEQ ID NO: 678 - DNA, 15 bp, Artificial Sequence
  Synthetic oligonucleotideCombined DNA/RNA Molecule nucleotides 5 to 12 are deoxyribonucleotides-other nucleotides are ribonucleotides
  modified_base (1)..(1): 2 ′-O,4 ′-C-methylene- linked bicyclic ribonucleotide
  modified_base (2)..(2): 2 ′-O,4 ′-C-methylene- linked bicyclic ribonucleotide
  modified_base (3)..(3): 2 ′-O,4 ′-C-methylene- linked bicyclic ribonucleotide
  modified_base (4)..(4): 2 ′-O,4 ′-C-methylene- linked bicyclic ribonucleotide
  modified_base (13)..(13): 2 ′-O,4 ′-C-methylene- linked bicyclic ribonucleotide
  modified_base (14)..(14): 2 ′-O,4 ′-C-methylene- linked bicyclic ribonucleotide
  modified_base (15)..(15): 2 ′-O,4 ′-C-methylene- linked bicyclic ribonucleotide
aaggacatactctgg

SEQ ID NO: 679 - DNA, 15 bp, Artificial Sequence
  Synthetic oligonucleotideCombined DNA/RNA Molecule nucleotides 5 to 11 are deoxyribonucleotides-other nucleotides are ribonucleotides
  modified_base (1)..(1): 2 ′-O,4 ′-C-methylene- linked bicyclic ribonucleotide
  modified_base (2)..(2): 2 ′-O,4 ′-C-methylene- linked bicyclic ribonucleotide
  modified_base (3)..(3): 2 ′-O,4 ′-C-methylene- linked bicyclic ribonucleotide
  modified_base (4)..(4): 2 ′-O,4 ′-C-methylene- linked bicyclic ribonucleotide
  modified_base (12)..(12): 5-methylcytosine; 2 ′-O,4 ′-C-methylene- linked bicyclic ribonucleotide
  modified_base (13)..(13): 2 ′-O,4 ′-C-methylene- linked bicyclic ribonucleotide
  modified_base (14)..(14): 2 ′-O,4 ′-C-methylene- linked bicyclic ribonucleotide
  modified_base (15)..(15): 2 ′-O,4 ′-C-methylene- linked bicyclic ribonucleotide
aaggacatactctgg

SEQ ID NO: 680 - DNA, 16 bp, Artificial Sequence
  Synthetic oligonucleotideCombined DNA/RNA Molecule nucleotides 5 to 12 are deoxyribonucleotides-other nucleotides are ribonucleotides
  modified_base (1)..(1): 2 ′-O,4 ′-C-methylene- linked bicyclic ribonucleotide
  modified_base (2)..(2): 2 ′-O,4 ′-C-methylene- linked bicyclic ribonucleotide
  modified_base (3)..(3): 2 ′-O,4 ′-C-methylene- linked bicyclic ribonucleotide
  modified_base (4)..(4): 2 ′-O,4 ′-C-methylene- linked bicyclic ribonucleotide
  modified_base (13)..(13): 5-methylcytosine; 2 ′-O,4 ′-C-methylene- linked bicyclic ribonucleotide
  modified_base (14)..(14): 2 ′-O,4 ′-C-methylene- linked bicyclic ribonucleotide
  modified_base (15)..(15): 2 ′-O,4 ′-C-methylene- linked bicyclic ribonucleotide
  modified_base (16)..(16): 2 ′-O,4 ′-C-methylene- linked bicyclic ribonucleotide
aaaggacatactctgg

(19) United States
(12) Patent Application Publication
(10) Pub. No.: US 2018/0208926 A1
(43) Pub. Date: Jul. 26, 2018

(54) ANTISENSE OLIGONUCLEOTIDE INHIBITING ß2GPI EXPRESSION

(71) Applicant: Kyowa Hakko Kirin Co., Ltd., Tokyo (JP)

(72) Inventors: Yoji YAMADA, Tokyo (JP); Hiroto IWAI, Tokyo (JP); Kazuhiro MASUDA, Tokyo (JP)

(73) Assignee: Kyowa Hakko Kirin Co., Ltd., Tokyo (JP)

(21) Appl. No.: 15/744,251

(22) PCT Filed: Jul. 13, 2016
(86) PCT No.: PCT/JP2016/070642

§ 371 Date: Jan. 12, 2018

(30) Foreign Application Priority Data
Jul. 13, 2015 (JP) 2015-140081

(51) Int. Cl. C12N 15/113

(52) U.S. Cl. CPC C12N 15/113; C12N 2310/11

(57) ABSTRACT

The present invention aims to provide a novel nucleic acid capable of suppressing expression of β2GPI, as well as a pharmaceutical composition for the prophylaxis or treatment of diseases associated with β2GPI expression. The present invention solves the above-mentioned problem by providing an antisense oligonucleotide having a β2GPI expression suppressive activity, a pharmaceutical composition containing the antisense oligonucleotide, and a prophylactic or therapeutic drug for autoimmune diseases including APS, SLE and the like, and thrombosis in hemodialysis that contains the antisense oligonucleotide and suppresses β2GPI expression.

### TECHNICAL FIELD

The present invention relates to an antisense oligonucleotide for use in suppressing expression of β2GPI, or a pharmaceutical composition comprising the antisense oligonucleotide.

### BACKGROUND ART

β2-Glycoprotein 1 (β2GPI) (also referred to as apolipoprotein H, apoH) is a soluble glycoprotein consisting of 326 amino acid residues, and is mainly produced in the liver (International Journal of Clinical and Laboratory Research, 1992, vol. 21, p 256-263). β2GPI is considered to have many different kinds of physiological actions, and has been reported to be involved in platelet aggregation reaction, coagulation and fibrinolysis reaction, and oxidized LDL uptake in macrophages (non-patent document 1).

As for the association with diseases, it is known that β2GPI is a major corresponding antigen to antiphospholipid antibody that emerges in autoimmune diseases such as antiphospholipid antibody syndrome (APS) and systemic lupus erythematosus (SLE). Anti-β2GPI antibody is also deeply involved in the pathology formation in diseases, and it has also been revealed by researches using animal models and clinical researches that a complex formed by β2GPI and anti-β2GPI antibodies generates activation signals in membrane receptors of various cells such as vascular endothelial cell, monocyte, platelet, and trophoblast and, as a result, can induce pathology characteristic of APS, such as thrombosis and abnormal pregnancy (non-patent document 2). It is expected that the above-mentioned diseases can be prevented or treated by specifically inhibiting the formation of immune complex consisting of β2GPI and anti-β2GPI antibodies. However, since β2GPI is present in blood at a comparatively high concentration of 50-500 μg/mL, it is not easy to continuously inhibit all such β2GPI with, for example, general antibody drugs (non-patent document 3).

On the other hand, as a method of suppressing expression itself of genes, for example, an antisense method is known (patent document 1). To be specific, an oligonucleotide complementary to mRNA or mRNA precursor and the like of the target gene (antisense oligonucleotide) forms a double strand with the mRNA or mRNA precursor of the target gene when introduced into the cell, and can specifically suppress expression of the target gene. As a method of suppressing expression of gene other than the antisense method, a method utilizing, for example, RNA interference (hereinafter to be referred to as RNAi) and the like are known. With this method, expression of a target gene can be specifically suppressed by introducing a double-stranded RNA (siRNA) having the same sequence as the target gene (patent document 2).

While a part of the siRNA sequences targeting human β2GPI has been disclosed (patent document 3, 4), suppression of the expression of the gene is not known, nor is it known that antisense oligonucleotide suppresses expression of human β2GPI gene.

### DOCUMENT LIST

### Patent Documents

patent document 1: WO 98/56905
patent document 2: WO 2001/75164
patent document 3: WO 2005/116204
patent document 4: WO 2008/043561

### Non-Patent Documents

non-patent document 1: Ann. N. Y. Acad. Sci., 1285, 44-58 (2013)
non-patent document 2: N. Engl. J. Med., 368, 1033-1044 (2013)
non-patent document 3: J. Thromb. Haemost., 9, 1275-1284 (2011)

### SUMMARY OF THE INVENTION

### Problems to be Solved by the Invention

The present invention aims to provide an antisense oligonucleotide capable of suppressing expression of β2GPI. The present invention also aims to provide a pharmaceutical composition for the prophylaxis or treatment of diseases associated with β2GPI expression.

### Means of Solving the Problems

The present invention relates to the following (1)-(17).

(1) An antisense oligonucleotide consisting of 8-80 bases in length that suppresses an expression of β2GPI, comprising a sequence hybridizable to a nucleic acid consisting of a base sequence shown in any of SEQ ID NOs: 201-399 (“target base sequence” described in Table 1-1-1-4) under stringent conditions.
(2) An antisense oligonucleotide consisting of 8-80 bases in length that suppresses an expression of β2GPI, comprising at least 8 continuous bases in a base sequence shown in any of SEQ ID NOs: 2-200 (“antisense base sequence” described in Table 1-1-1-4).
(3) An antisense oligonucleotide consisting of 8-80 bases in length and complementary to a base sequence shown in any of SEQ ID NOs: 201-399 (base sequence selected from the group described in “target base sequence” of Table 1-1-1-4).
(4) The antisense oligonucleotide of (3), comprising a base sequence shown in any of SEQ ID NOs: 2-200 (base sequence selected from the group described in “antisense base sequence” of Table 1-1-1-4).
(5) The antisense oligonucleotide of (3), comprising a base sequence shown in any of SEQ ID NOs: 2-200 wherein 1 or several bases are deleted, substituted or added.
(6) An antisense oligonucleotide consisting of a base sequence shown in any of SEQ ID NOs: 2-200.
(7) An antisense oligonucleotide consisting of a base sequence shown in any of SEQ ID NOs: 400-680.
(8) The antisense oligonucleotide of any one of (1)-(7), wherein the 5′-terminal vicinity and/or the 3′-terminal vicinity are/is constituted of a sugar moiety-modified nucleotide.
(9) The antisense oligonucleotide of any one of (1)-(8), comprising a ligand.
(10) A pharmaceutical composition comprising the antisense oligonucleotide of any one of (1)-(9).
(11) The pharmaceutical composition of (10) for the treatment or prophylaxis of an autoimmune disease or thrombosis.
(12) A method of treating a disorder mediated by an anti-β2GPI antibody, comprising a step of administering a therapeutically effective amount of the antisense oligonucleotide of any one of (1)-(9) or the pharmaceutical composition of (10) or (11) to a human in need of such treatment.
(13) The method of (12), wherein the aforementioned disorder is an autoimmune disease or thrombosis.
(14) Use of the antisense oligonucleotide of any one of (1)-(9) for the manufacture of a pharmaceutical composition for preventing or treating a disorder mediated by an anti-β2GPI antibody.
(15) The use of (14), wherein the aforementioned disorder is an autoimmune disease or thrombosis.
(16) The antisense oligonucleotide of any one of (1)-(9) for use in the prophylaxis or treatment of a disorder mediated by an anti-β2GPI antibody.
(17) The antisense nucleotide of (16), wherein the aforementioned disorder is an autoimmune disease or thrombosis.

### Effect of the Invention

Expression of β2GPI can be suppressed by administering the antisense oligonucleotide of the present invention or a pharmaceutical composition comprising the antisense oligonucleotide. The antisense oligonucleotide or a pharmaceutical composition comprising the antisense oligonucleotide of the present invention is useful for the prophylaxis or treatment of a disease associated with the expression of β2GPI, particularly a disorder mediated by an anti-β2GPI antibody.

### DESCRIPTION OF EMBODIMENTS

As a gene encoding β2GPI (hereinafter to be also referred to as β2GPI gene), which is targeted by the antisense oligonucleotide of the present invention, a cDNA base sequence (SEQ ID NO: 1) corresponding to a full length mRNA of β2GPI, which is registered as Genbank Accession No. NM_000042, can be mentioned.

### 1. Antisense Oligonucleotide of the Present Invention

In the present invention, the antisense oligonucleotide refers to an oligonucleotide complementary to a DNA encoding the target gene and to mRNA precursor and mRNA transcribed from such DNA. It suppresses the actions (transcription, editing after transcription, translation and the like) of DNA, mRNA precursor or mRNA, which is targeted by the antisense oligonucleotide, by forming a double strand or triple strand with the DNA, mRNA precursor or mRNA. The antisense oligonucleotide includes not only those completely complementary to DNA, mRNA precursor or mRNA to be the target, but also those containing one or several mismatches as long as they can hybridize to the DNA, mRNA precursor or mRNA under stringent conditions. The antisense oligonucleotide of the present invention may be introduced into the form of a hairpin oligomer or cyclic oligomer as long as it is nucleic acid that hybridizes to the target gene. The antisense oligonucleotide may contain a structural element such as an internal or terminal bulge or loop, and the like.

The present invention provides an antisense oligonucleotide that suppresses expression of β2GPI (to be also referred to as the antisense oligonucleotide of the present invention in the present specification).

The antisense oligonucleotide of the present invention may be any molecule as long as it is a molecule in which a nucleotide or a molecule having functions equivalent to those of the nucleotide is polymerized. Examples thereof include DNA that is a polymer of deoxyribonucleotide, RNA that is a polymer of ribonucleotide, chimeric nucleic acid composed of RNA and DNA, and nucleotide polymer in which at least one nucleotide of these nucleic acids is substituted by a molecule having functions equivalent to those of the nucleotide. Uracil (U) in RNA can be unambiguously read as thymine (T) in DNA.

Examples of the molecule having functions equivalent to those of the nucleotide include nucleotide derivatives and the like. The nucleotide derivative may be any molecule as long as it is a molecule obtained by modifying a nucleotide. For example, a molecule obtained by modifying deoxyribonucleotide or ribonucleotide and the like to improve or stabilize nuclease resistance, enhance affinity for complementary strand nucleic acid, enhance cell permeability or visualize same, as compared with DNA or RNA, are preferably used.

Examples of the molecule obtained by modifying a nucleotide include sugar moiety-modified nucleotide, phosphodiester bond-modified nucleotide, base-modified nucleotide, nucleotide in which at least one of a sugar moiety, a phosphodiester bond and a base is modified, and the like.

While the sugar moiety-modified nucleotide may be any as long as the chemical structure of the sugar of nucleotide is partly or entirely modified or substituted by any substituent, or substituted by any atom, 2′-modified nucleotide is preferably used.

Examples of the 2′-modified nucleotide include a 2′-modified nucleotide in which the 2′—OH group of ribose is substituted by a substituent selected from R, R′OR, SH, SR, NH₂, NHR, NR₂, N₃, CN, F, Cl, Br and I (R is alkyl or aryl, preferably alkyl having 1-6 carbon atoms, R′ is alkylene, preferably alkylene having 1-6 carbon atoms), and preferable examples of the substituent include F and methoxy group. In addition, preferable examples of the substituent include a 2′-modified nucleotide substituted by a substituent selected from the group consisting of 2-(methoxy)ethoxy group, 3-aminopropoxy group, 2-[(N,N-dimethylamino)oxy]ethoxy group, 3-(N,N-dimethylamino)propoxy group, 2-[2-(N,N-dimethylamino)ethoxy]ethoxy group, 2-(methylamino)-2-oxoethoxy group, 2-(N-methylcarbamoyl)ethoxy group and 2-cyanoetoxy group, and the like. More preferable examples include 2′-modified nucleotide substituted by a substituent selected from the group consisting of methoxy group and 2-(methoxy)ethoxy group and the like.

As the sugar moiety-modified nucleotide, a crosslinking structure type artificial nucleic acid having two cyclic structures by introducing a crosslinking structure into the sugar moiety (Bridged Nucleic Acid) (BNA) can be used preferably. Specific examples thereof include locked artificial nucleic acid wherein the 2′-position oxygen atom and the 4′-position carbon atom are crosslinked via methylene (Locked Nucleic Acid) (LNA) [Tetrahedron Letters, 38, 8735, (1997) and Tetrahedron, 54, 3607, (1998)], ethylene crosslinking structure type artificial nucleic acid (Ethylene bridged nucleic acid) (ENA) [Nucleic Acid Research, 32, e175(2004)], Constrained Ethyl (cEt) [The Journal of Organic Chemistry 75, 1569 (2010)], Amido-Bridged Nucleic Acid (AmNA) [Chem Bio Chem 13, 2513 (2012)], and 2′-O,4′-C-Spirocyclopropylene bridged nucleic acid (scpBNA) [Chem. Commun., 51, 9737 (2015)] and the like.

Furthermore, peptide nucleic acid (PNA) [Acc. Chem. Res., 32, 624 (1999)], oxy-peptide nucleic acid (OPNA) [J. Am. Chem. Soc., 123, 4653 (2001)], peptide ribonucleic acid (PRNA) [J. Am. Chem. Soc., 122, 6900 (2000)] and the like can also be mentioned as the sugar moiety-modified nucleotide.

The phosphodiester bond-modified nucleotide may be any as long as the chemical structure of the phosphodiester bond is partly or entirely modified or substituted by any substituent, or substituted by any atom. Examples thereof include a nucleotide in which the phosphodiester bond is substituted by a phosphorothioate bond, a nucleotide in which the phosphodiester bond is substituted by a phosphorodithioate bond, a nucleotide in which the phosphodiester bond is substituted by an alkylphosphonate bond, a nucleotide in which the phosphodiester bond is substituted by a phosphoramidate bond and the like. Preferably, a nucleotide in which the phosphodiester bond is substituted by a phosphorothioate bond can be mentioned.

The base-modified nucleotide may be any as long as the chemical structure of the base of the nucleotide is partly or entirely modified or substituted by any substituent, or substituted by any atom. Examples thereof include one in which an oxygen atom in the base is substituted by a sulfur atom, one in which a hydrogen atom is substituted by an alkyl group having 1-6 carbon atoms, a halogen group, one in which a methyl group is substituted by hydrogen, hydroxymethyl, alkyl group having 2-6 carbon atoms, and one in which an amino group is substituted by alkyl group having 1-6 carbon atoms, alkanoyl group having 1-6 carbon atoms, oxo group, a hydroxy group, and the like. Use of 5-methylcytosine (5-mC) as a base-modified nucleotide instead of cytosine (C) is also one of the preferable forms of the present invention.

As the nucleotide derivative, one obtained by adding other chemical substance such as lipids such as cholesterol, fatty acid, tocopherol, retinoid and the like, saccharides such as N-acetylgalactosamine (GalNAc), galactose (Gal), mannose (Man) and the like, antibodies such as full antibody, Fab (Fragment antigen-binding antibody), scFv (Single-chain variable fragment antibody) and VHH (variable domain of heavy chain antibody) and the like, proteins such as low-density lipoprotein (LDL), human serum albumin and the like, peptides such as RGD, NGR, R9, CPP (cell penetrating peptide) and the like, small molecules such as phenazine, phenanthridine, anthraquinone, folic acid and the like, synthetic polymers such as synthetic polyamino acid and the like, nucleic acid aptamers, dye such as acridine, fluorescein, rhodamine, coumarin and the like, fluorophore such as Cy3 series, Alexa series, black hole quencher and the like, and the like, directly or via a linker, to a nucleotide or a nucleotide derivative wherein at least one of sugar moiety, phosphodiester bond and base is modified can also be mentioned. Specific examples thereof include polyamine-added nucleotide derivative, cholesterol-added nucleotide derivative, steroid-added nucleotide derivative, GalNAc-added nucleotide derivative, bile acid-added nucleotide derivative, fatty acid-added nucleotide derivative, vitamin-added nucleotide derivative, Cy5-added nucleotide derivative, Cy3-added nucleotide derivative, 6-FAM-added nucleotide derivative, and biotin-added nucleotide derivative and the like, preferably GalNAc-added nucleotide derivative can be mentioned. These can be modified at the 5′-terminal, 3′-terminal and/or inside of the sequence by reacting a modifier reactive on the solid phase, during elongation reaction on the solid phase. Alternatively, a nucleic acid into which a functional group such as an amino group, a mercapto group, an azide group, a triple bond and the like has been introduced may be previously synthesized and purified, and reacted with a modifier.

The nucleotide derivative may form a crosslinking structure, such as alkylene structure, peptide structure, nucleotide structure, ether structure, ester structure, a structure of a combination of at least one of these and the like, with other nucleotide or nucleotide derivative in the nucleic acid.

The antisense oligonucleotide of the present invention also encompasses an antisense oligonucleotide wherein the atoms in a molecule are partly or entirely substituted by an atom (isotope) having a different mass number.

In the present specification, “complement” means a relationship forming a base pairing between two bases, and refers to a double helix structure as a whole double-stranded region via a loose hydrogen bond, for example, the relationship between adenine and thymine or uracil, and the relationship between guanine and cytosine.

The length of the antisense oligonucleotide of the present invention is 8-80 bases, preferably 8-30 bases. For example, it may be 8-20 bases, 10-20 bases, 13-20 bases, 13-16 bases, 13 bases, 14 bases, 15 bases, 16 bases, 17 bases, 18 bases, 19 bases or 20 bases.

While particular preferable specific base sequences are described in the present DESCRIPTION, an antisense oligonucleotide 8-80 bases in length and containing at least 8 continuous bases selected from the antisense base sequences described in Tables 1-1 to 1-4 is also a preferable antisense oligonucleotide. Antisense oligonucleotide 8-80 bases, preferably 8-30 bases, in length and containing more preferably 9 or more, further preferably 10 or more, still more preferably 11 or more, particularly preferably 12 or more, most preferably 13, continuous bases selected from the antisense base sequences described in Table 1 are also preferable antisense oligonucleotides.

TABLE 1-1

| | antisense base | | target base |
|---|---|---|---|
| SEQ ID NO: | sequence | SEQ ID NO: | sequence |
| 2 | GAGCACTGGAGAA | 201 | TTCTCCAGTGCTC |
| 3 | TGAGCACTGGAGA | 202 | TCTCCAGTGCTCA |
| 4 | AGATGAGCACTGG | 203 | CCAGTGCTCATCT |
| 5 | AGCAACATGGCAG | 204 | CTGCCATGTTGCT |
| 6 | TAGCAACATGGCA | 205 | TGCCATGTTGCTA |
| 7 | ATAGCAACATGGC | 206 | GCCATGTTGCTAT |
| 8 | CAATAGCAACATG | 207 | CATGTTGCTATTG |
| 9 | GCAATAGCAACAT | 208 | ATGTTGCTATTGC |
| 10 | CCTGCAATAGCAA | 209 | TTGCTATTGCAGG |
| 11 | TCCTGCAATAGCA | 210 | TGCTATTGCAGGA |
| 12 | GTCCTGCAATAGC | 211 | GCTATTGCAGGAC |
| 13 | TCCGTCCTGCAAT | 212 | ATTGCAGGACGGA |
| 14 | GTCCGTCCTGCAA | 213 | TTGCAGGACGGAC |
| 15 | GGTCCGTCCTGCA | 214 | TGCAGGACGGACC |
| 16 | AGGTCCGTCCTGC | 215 | GCAGGACGGACCT |
| 17 | CAGGTCCGTCCTG | 216 | CAGGACGGACCTG |
| 18 | TGGGACAGGTCCG | 217 | CGGACCTGTCCCA |
| 19 | TTGGGACAGGTCC | 218 | GGACCTGTCCCAA |
| 20 | GGCTTGGGACAGG | 219 | CCTGTCCCAAGCC |
| 21 | TGGCTTGGGACAG | 220 | CTGTCCCAAGCCA |
| 22 | CTGGCTTGGGACA | 221 | TGTCCCAAGCCAG |
| 23 | TCTGGCTTGGGAC | 222 | GTCCCAAGCCAGA |
| 24 | CATCTGGCTTGGG | 223 | CCCAAGCCAGATG |
| 25 | TCATCTGGCTTGG | 224 | CCAAGCCAGATGA |
| 26 | ATCATCTGGCTTG | 225 | CAAGCCAGATGAT |
| 27 | AATCATCTGGCTT | 226 | AAGCCAGATGATT |
| 28 | AAATCATCTGGCT | 227 | AGCCAGATGATTT |
| 29 | TCCTGGCTCATAG | 228 | CTATGAGCCAGGA |
| 30 | CTCCTGGCTCATA | 229 | TATGAGCCAGGAG |
| 31 | TCTCCTGGCTCAT | 230 | ATGAGCCAGGAGA |
| 32 | CCGGCTTGCAGGA | 231 | TCCTGCAAGCCGG |
| 33 | CCCGGCTTGCAGG | 232 | CCTGCAAGCCGGG |
| 34 | GCCCGGCTTGCAG | 233 | CTGCAAGCCGGGC |
| 35 | AGCCCGGCTTGCA | 234 | TGCAAGCCGGGCT |
| 36 | TAGCCCGGCTTGC | 235 | GCAAGCCGGGCTA |
| 37 | ATAGCCCGGCTTG | 236 | CAAGCCGGGCTAT |
| 38 | CATAGCCCGGCTT | 237 | AAGCCGGGCTATG |
| 39 | ACACATAGCCCGG | 238 | CCGGGCTATGTGT |
| 40 | GACACATAGCCCG | 239 | CGGGCTATGTGTC |
| 41 | TGTGAGAGGGCAG | 240 | CTGCCCTCTCACA |
| 42 | TTGATGGGCCACA | 241 | TGTGGCCCATCAA |
| 43 | GTTGATGGGCCAC | 242 | GTGGCCCATCAAC |
| 44 | TGTTGATGGGCCA | 243 | TGGCCCATCAACA |
| 45 | GTGTTGATGGGCC | 244 | GGCCCATCAACAC |
| 46 | GGACATACTCTGG | 245 | CCAGAGTATGTCC |
| 47 | AGGACATACTCTG | 246 | CAGAGTATGTCCT |
| 48 | AAGGACATACTCT | 247 | AGAGTATGTCCTT |
| 49 | AAAGGACATACTC | 248 | GAGTATGTCCTTT |
| 50 | GCTCCATTTTCTA | 249 | TAGAAAATGGAGC |
| 51 | GGGATATTCAAAA | 250 | TTTTGAATATCCC |
| 52 | TGGGATATTCAAA | 251 | TTTGAATATCCCA |
| 53 | TTGGGATATTCAA | 252 | TTGAATATCCCAA |
| 54 | GTTGGGATATTCA | 253 | TGAATATCCCAAC |
| 55 | GCACTTGGCAGAA | 254 | TTCTGCCAAGTGC |
| 56 | TGCACTTGGCAGA | 255 | TCTGCCAAGTGCA |
| 57 | GTGCACTTGGCAG | 256 | CTGCCAAGTGCAC |
| 58 | AGTGCACTTGGCA | 257 | TGCCAAGTGCACT |

TABLE 1-2

| | antisense base | | target base |
|---|---|---|---|
| SEQ ID NO: | sequence | SEQ ID NO: | sequence |
| 59 | CAGTGCACTTGGC | 258 | GCCAAGTGCACTG |
| 60 | CTCAGTGCACTTG | 259 | CAAGTGCACTGAG |
| 61 | CCTCAGTGCACTT | 260 | AAGTGCACTGAGG |
| 62 | TCCTCAGTGCACT | 261 | AGTGCACTGAGGA |
| 63 | TTCCTCAGTGCAC | 262 | GTGCACTGAGGAA |
| 64 | CTTCCTCAGTGCA | 263 | TGCACTGAGGAAG |
| 65 | CCTTCCTCAGTGC | 264 | GCACTGAGGAAGG |
| 66 | TCCTTCCTCAGTG | 265 | CACTGAGGAAGGA |
| 67 | GCTGATGGCTTAT | 266 | ATAAGCCATCAGC |
| 68 | AGCTGATGGCTTA | 267 | TAAGCCATCAGCT |
| 69 | CAGCTGATGGCTT | 268 | AAGCCATCAGCTG |
| 70 | CCAGCTGATGGCT | 269 | AGCCATCAGCTGG |
| 71 | TCCAGCTGATGGC | 270 | GCCATCAGCTGGA |
| 72 | TTCCAGCTGATGG | 271 | CCATCAGCTGGAA |
| 73 | GTTTCCAGCTGAT | 272 | ATCAGCTGGAAAC |
| 74 | TGTTTCCAGCTGA | 273 | TCAGCTGGAAACA |
| 75 | TTGTTTCCAGCTG | 274 | CAGCTGGAAACAA |
| 76 | AACTGCTGTGTCC | 275 | GGACACAGCAGTT |
| 77 | AAAAACTGCTGTG | 276 | CACAGCAGTTTTT |
| 78 | TGGCAAACATTCA | 277 | TGAATGTTTGCCA |
| 79 | GTGGCAAACATTC | 278 | GAATGTTTGCCAC |
| 80 | GTTGTGGCAAACA | 279 | TGTTTGCCACAAC |
| 81 | TGTTGTGGCAAAC | 280 | GTTTGCCACAACA |
| 82 | GCATGTTGTGGCA | 281 | TGCCACAACATGC |
| 83 | CGCATGTTGTGGC | 282 | GCCACAACATGCG |
| 84 | TCGCATGTTGTGG | 283 | CCACAACATGCGA |
| 85 | ATCGCATGTTGTG | 284 | CACAACATGCGAT |
| 86 | ACATCGCATGTTG | 285 | CAACATGCGATGT |
| 87 | TTCCAAACATCGC | 286 | GCGATGTTTGGAA |
| 88 | AGTCCAATTTCCA | 287 | TGGAAATTGGACT |
| 89 | CCCTGCATTCTGG | 288 | CCAGAATGCAGGG |
| 90 | TCCCTGCATTCTG | 289 | CAGAATGCAGGGA |
| 91 | TTCCCTGCATTCT | 290 | AGAATGCAGGGAA |
| 92 | ATTGTCTGGTCTT | 291 | AAGACCAGACAAT |
| 93 | CATTGTCTGGTCT | 292 | AGACCAGACAATG |
| 94 | CCATTGTCTGGTC | 293 | GACCAGACAATGG |
| 95 | TCCATTGTCTGGT | 294 | ACCAGACAATGGA |
| 96 | ATCCATTGTCTGG | 295 | CCAGACAATGGAT |
| 97 | AATCCATTGTCTG | 296 | CAGACAATGGATT |
| 98 | GCAGGATAGTTCA | 297 | TGAACTATCCTGC |
| 99 | GGTTTTGCAGGAT | 298 | ATCCTGCAAAACC |
| 100 | TGGTTTTGCAGGA | 299 | TCCTGCAAAACCA |
| 101 | GGCTTTATCCTTG | 300 | CAAGGATAAAGCC |
| 102 | GTGGCTTTATCCT | 301 | AGGATAAAGCCAC |
| 103 | ATGTGGCTTTATC | 302 | GATAAAGCCACAT |
| 104 | AATGTGGCTTTAT | 303 | ATAAAGCCACATT |
| 105 | CCAAATGTGGCTT | 304 | AAGCCACATTTGG |
| 106 | CATCATGGCAGCC | 305 | GGCTGCCATGATG |
| 107 | CCATCATGGCAGC | 306 | GCTGCCATGATGG |
| 108 | TCCATCATGGCAG | 307 | CTGCCATGATGGA |
| 109 | ATCCATCATGGCA | 308 | TGCCATGATGGAT |
| 110 | TATCCATCATGGC | 309 | GCCATGATGGATA |
| 111 | ATATCCATCATGG | 310 | CCATGATGGATAT |
| 112 | CCCAGTTTGGTAC | 311 | GTACCAAACTGGG |
| 113 | TCCCAGTTTGGTA | 312 | TACCAAACTGGGA |
| 114 | TTCCCAGTTTGGT | 313 | ACCAAACTGGGAA |
| 115 | TTTCCCAGTTTGG | 314 | CCAAACTGGGAAA |

TABLE 1-3

| | antisense base | | target base |
|---|---|---|---|
| SEQ ID NO: | sequence | SEQ ID NO: | sequence |
| 116 | GTTTCCCAGTTTG | 315 | CAAACTGGGAAAC |
| 117 | CAGTTTCCCAGTT | 316 | AACTGGGAAACTG |
| 118 | CCAGTTTCCCAGT | 317 | ACTGGGAAACTGG |
| 119 | ACCAGTTTCCCAG | 318 | CTGGGAAACTGGT |
| 120 | GACCAGTTTCCCA | 319 | TGGGAAACTGGTC |
| 121 | AGACCAGTTTCCC | 320 | GGGAAACTGGTCT |
| 122 | GCAGACCAGTTTC | 321 | GAAACTGGTCTGC |
| 123 | GGCAGACCAGTTT | 322 | AAACTGGTCTGCC |
| 124 | TGGCAGACCAGTT | 323 | AACTGGTCTGCCA |
| 125 | ATGGCAGACCAGT | 324 | ACTGGTCTGCCAT |
| 126 | CATGGCAGACCAG | 325 | CTGGTCTGCCATG |
| 127 | GCATGGCAGACCA | 326 | TGGTCTGCCATGC |
| 128 | GGCATGGCAGACC | 327 | GGTCTGCCATGCC |
| 129 | TGGCATGGCAGAC | 328 | GTCTGCCATGCCA |
| 130 | TTGGCATGGCAGA | 329 | TCTGCCATGCCAA |
| 131 | CTTGGCATGGCAG | 330 | CTGCCATGCCAAG |
| 132 | ACTTGGCATGGCA | 331 | TGCCATGCCAAGT |
| 133 | AACTTGGCATGGC | 332 | GCCATGCCAAGTT |
| 134 | CAACTTGGCATGG | 333 | CCATGCCAAGTTG |
| 135 | ACAACTTGGCATG | 334 | CATGCCAAGTTGT |
| 136 | GCTTTACAACTTG | 335 | CAAGTTGTAAAGC |
| 137 | TCACAGGTACTTT | 336 | AAAGTACCTGTGA |
| 138 | ACACCACAGTGGC | 337 | GCCACTGTGGTGT |
| 139 | GTACACCACAGTG | 338 | CACTGTGGTGTAC |
| 140 | GGTACACCACAGT | 339 | ACTGTGGTGTACC |
| 141 | TGGTACACCACAG | 340 | CTGTGGTGTACCA |
| 142 | TTGGTACACCACA | 341 | TGTGGTGTACCAA |
| 143 | CTTGGTACACCAC | 342 | GTGGTGTACCAAG |
| 144 | CCTTGGTACACCA | 343 | TGGTGTACCAAGG |
| 145 | TCCTTGGTACACC | 344 | GGTGTACCAAGGA |
| 146 | CTCCTTGGTACAC | 345 | GTGTACCAAGGAG |
| 147 | TCTCCTTGGTACA | 346 | TGTACCAAGGAGA |
| 148 | CTCTCCTTGGTAC | 347 | GTACCAAGGAGAG |
| 149 | TCTCTCCTTGGTA | 348 | TACCAAGGAGAGA |
| 150 | CTCTCTCCTTGGT | 349 | ACCAAGGAGAGAG |
| 151 | TCTCTCTCCTTGG | 350 | CCAAGGAGAGAGA |
| 152 | CTCTCTCTCCTTG | 351 | CAAGGAGAGAGAG |
| 153 | ACTCTCTCTCCTT | 352 | AAGGAGAGAGAGT |
| 154 | TACTCTCTCTCCT | 353 | AGGAGAGAGAGTA |
| 155 | TTACTCTCTCTCC | 354 | GGAGAGAGAGTAA |
| 156 | GCATTCCATTCTT | 355 | AAGAATGGAATGC |
| 157 | AGCATTCCATTCT | 356 | AGAATGGAATGCT |
| 158 | AGCTACACTTCTT | 357 | AAGAAGTGTAGCT |
| 159 | TAGCTACACTTCT | 358 | AGAAGTGTAGCTA |
| 160 | TTGAAGCATTTGG | 359 | CCAAATGCTTCAA |
| 161 | GTTCCTTGAAGCA | 360 | TGCTTCAAGGAAC |
| 162 | TGTTCCTTGAAGC | 361 | GCTTCAAGGAACA |
| 163 | GTGTTCCTTGAAG | 362 | CTTCAAGGAACAC |
| 164 | TGTGTTCCTTGAA | 363 | TTCAAGGAACACA |
| 165 | CTGTGTTCCTTGA | 364 | TCAAGGAACACAG |
| 166 | ACTGTGTTCCTTG | 365 | CAAGGAACACAGT |
| 167 | AACTGTGTTCCTT | 366 | AAGGAACACAGTT |
| 168 | GAACTGTGTTCCT | 367 | AGGAACACAGTTC |
| 169 | AGAACTGTGTTCC | 368 | GGAACACAGTTCT |
| 170 | GAGAACTGTGTTC | 369 | GAACACAGTTCTC |
| 171 | AGAGAACTGTGTT | 370 | AACACAGTTCTCT |
| 172 | CAGAGAACTGTGT | 371 | ACACAGTTCTCTG |

TABLE 1-4

| | antisense base | | target base |
|---|---|---|---|
| SEQ ID NO: | sequence | SEQ ID NO: | sequence |
| 173 | CCAGAGAACTGTG | 372 | CACAGTTCTCTGG |
| 174 | GCCAGAGAACTGT | 373 | ACAGTTCTCTGGC |
| 175 | AGCCAGAGAACTG | 374 | CAGTTCTCTGGCT |
| 176 | AAGCCAGAGAACT | 375 | AGTTCTCTGGCTT |
| 177 | GCATCAGTTTTCC | 376 | GGAAAACTGATGC |
| 178 | TGCATCAGTTTTC | 377 | GAAAACTGATGCA |
| 179 | GATGCATCAGTTT | 378 | AAACTGATGCATC |
| 180 | GGATGCATCAGTT | 379 | AACTGATGCATCC |
| 181 | CGGATGCATCAGT | 380 | ACTGATGCATCCG |
| 182 | TCGGATGCATCAG | 381 | CTGATGCATCCGA |
| 183 | ATCGGATGCATCA | 382 | TGATGCATCCGAT |
| 184 | CATCGGATGCATC | 383 | GATGCATCCGATG |
| 185 | ACATCGGATGCAT | 384 | ATGCATCCGATGT |
| 186 | TACATCGGATGCA | 385 | TGCATCCGATGTA |
| 187 | TTACATCGGATGC | 386 | GCATCCGATGTAA |
| 188 | GCTTTACATCGGA | 387 | TCCGATGTAAAGC |
| 189 | GGCTTTACATCGG | 388 | CCGATGTAAAGCC |
| 190 | TGGCTTTACATCG | 389 | CGATGTAAAGCCA |
| 191 | TGTGGAATCTGAA | 390 | TTCAGATTCCACA |
| 192 | AGTGTGACATTTT | 391 | AAAATGTCACACT |
| 193 | AAGTGTGACATTT | 392 | AAATGTCACACTT |
| 194 | CCTTGGATGAACA | 393 | TGTTCATCCAAGG |
| 195 | TCCTTGGATGAAC | 394 | GTTCATCCAAGGA |
| 196 | GTTCCTTGGATGA | 395 | TCATCCAAGGAAC |
| 197 | GGTTCCTTGGATG | 396 | CATCCAAGGAACC |
| 198 | AGGTTCCTTGGAT | 397 | ATCCAAGGAACCT |
| 199 | TAGGTTCCTTGGA | 398 | TCCAAGGAACCTA |
| 200 | TTAGGTTCCTTGG | 399 | CCAAGGAACCTAA |

Typical preferable antisense oligonucleotides include oligonucleotides containing at least 8, more preferably 9 or more, further preferably 10 or more, still more preferably 11 or more, particularly preferably 12 or more, most preferably 13, continuous nucleic acid bases from the 5′-terminal of the antisense base sequences (sequences shown in any of SEQ ID NOs: 2-200) described in Tables 1-1 to 1-4. Similarly, preferable antisense oligonucleotides include oligonucleotides containing at least 8 or more, more preferably 9 or more, further preferably 10 or more, still more preferably 11 or more, particularly preferably 12 or more, most preferably 13, continuous nucleic acid bases from the 3′-terminal of the antisense base sequences described in Table 1.

As the antisense oligonucleotide of the present invention, the antisense oligonucleotides (sequences shown in any of SEQ ID NOs: 400-680) described in Table 2-1 to 2-3, Table 3-1 to 3-2 or 4 are preferably used.

As the antisense oligonucleotide of the present invention, antisense oligonucleotides which are the antisense oligonucleotides described in Table 2-1 to 2-3, Table 3-1 to 3-2 and 4 and containing antisense oligonucleotide sequences having a β2GPI relative expression level of 0.5 or less (e.g., sequences shown in any of SEQ ID NOs: 407, 408, 415, 418, 424, 427-430, 437, 440, 443, 445-454, 456-460, 462, 463, 465, 470, 471, 481-484, 492, 505, 506, 508, 509, 511-519, 523, 524-527, 529, 531-533, 535-537, 539, 542, 546-548, 550-554, 558-560, 565-569, 574, 575, 577, 578, 582-597, 599-641, 643-680) are more preferably used. Further preferably, antisense oligonucleotide sequences having a β2GPI relative expression level of 0.3 or less (e.g., sequences shown in any of SEQ ID NOs: 445, 450, 456, 457, 459, 462, 471, 482, 509, 513, 514, 518, 523, 525, 526, 529, 553, 565, 566, 567, 584, 585, 586-597, 599, 600, 602-611, 613, 615-621, 624-630, 633-641, 643, 644, 645, 647, 648, 649, 651-657, 659-680), particularly preferably, antisense oligonucleotide sequences having a β2GPI relative expression level of 0.1 or less (e.g., sequences shown in any of SEQ ID NOs: 586, 587, 588, 590, 591, 592, 593, 602, 603, 604, 605, 608, 617, 618, 620, 624, 625, 627, 636, 640, 641, 652, 656, 659, 660, 661, 662, 663, 664, 665, 666, 667, 668, 669, 671, 672, 673, 675), can be used.

As the antisense oligonucleotide of the present invention, a nucleic acid containing a nucleic acid composed of a base sequence complementary to a part of the target base sequence of a β2GPI gene and suppressing the expression of β2GPI is used. In the nucleic acid, 1-3 bases, preferably 1-2 bases, more preferably 1 base, may be deleted, substituted or added.

When the antisense oligonucleotide of the present invention is intracellularly introduced, it binds to complementary mRNA or complementary mRNA precursor and sterically inhibits translation of the mRNA or mRNA precursor into proteins, whereby expression of the β2GPI gene can be suppressed.

In addition, the intracellularly-introduced antisense oligonucleotide of the present invention sometimes binds to complementary mRNA or complementary mRNA precursor in the cells and cleaves the mRNA or mRNA precursor. As an example thereof, an action through RNaseH, which is an endonuclease that degrades RNA strand of RNA and DNA double strand, is known. When the antisense oligonucleotide of the present invention forms a double strand with mRNA and mRNA precursor in the cell, the double strand is recognized by the endogenous RNaseH and complementary mRNA strand can be enzymatically degraded.

To induce cleavage of mRNA and mRNA precursor by RNaseH, an antisense oligonucleotide having a DNA region of continuous 4-80 nucleotides is preferable. In this case, the antisense oligonucleotide preferably has a 0-80%, more preferably 10-60%, further preferably 20-50%, sugar moiety-modified nucleotide. When a sugar moiety-modified nucleotide is present, the DNA region more preferably consists of continuous 4-20 nucleotides, further preferably continuous 4-15 nucleotides, most preferably continuous 5-10 nucleotides. Furthermore, the position of the sugar moiety-modified nucleotide in the antisense oligonucleotide of the present invention is preferably in the vicinity of 5′-terminal and/or 3′-terminal, more preferably at a position within 30% of the total length from the 5′ end and/or a position within 30% of the total length from the 3′-terminal, further preferably at a position within 25% of the total length from the 5′ end and/or a position within 25% of the total length from the 3′-terminal.

The 5′-terminal vicinity and/or 3′-terminal vicinity of the antisense oligonucleotide of the present invention are/is particularly preferably constituted of a sugar moiety-modified nucleotide. In the present specification, the 5′-terminal vicinity being constituted of a sugar moiety-modified nucleotide means that 1-4, preferably 2-4, continuous nucleotides from the 5′-terminal are sugar moiety-modified nucleotides, and the 3′-terminal vicinity being constituted of a sugar moiety-modified nucleotide means that 1-4, preferably 2-4, continuous nucleotides from the 3′-terminal are sugar moiety-modified nucleotides. That is, as the antisense oligonucleotide of the present invention, an antisense oligonucleotide in which 1-4 nucleotides from the 5′-terminal thereof are sugar moiety-modified nucleotides is preferably used, and an antisense oligonucleotide in which 2-4 nucleotides from the 5′-terminal thereof are sugar moiety-modified nucleotides is more preferably used. As the antisense oligonucleotide of the present invention, an antisense oligonucleotide in which 1-4 nucleotides from the 3′-terminal thereof are sugar moiety-modified nucleotides is preferably used, and an antisense oligonucleotide in which 2-4 nucleotides from the 3′-terminal thereof are sugar moiety-modified nucleotides is more preferably used.

In the present invention, the stringent conditions mean conditions under which the antisense oligonucleotide of the present invention hybridizes to the target base sequence of the β2GPI gene but does not hybridize to other sequences, and even if it hybridizes, the amount thereof is drastically smaller than the amount of hybridization to the target base sequence, and only a relatively negligible trace amount. Such conditions can be easily selected by changing the temperature during hybridization reaction and washing, salt concentration of hybridization reaction mixture and washings and the like. Specifically, one embodiment of the stringent conditions includes, but is not limited to, hybridizing in 6×SSC (0.9 M NaCl, 0.09 M trisodium citrate) or 6×SSPE (3 M NaCl, 0.2 M NaH₂PO₄, 20 mM EDTA.2Na, pH 7.4) at 42° C., and further washing with 0.5×SSC at 42° C. As the hybridization method, Southern blot hybridization method and the like can be used. Specifically, hybridization can be performed according to the methods described in Molecular Cloning: A Laboratory Manual, Second Edition (1989) (Cold Spring Harbor Laboratory Press), Current Protocols in Molecular Biology (1994) (Wiley-Interscience) and the like.

A method of producing the antisense oligonucleotide of the present invention is not particularly limited, and a method using a known chemical synthesis, or an enzymatic transcription method and the like can be mentioned. As a method using a known chemical synthesis, a phosphoramidite method, a phosphorothioate method, a phosphotriester method, a CEM method [Nucleic Acid Research, 35, 3287 (2007)] and the like can be mentioned and, for example, it can be synthesized by ABI3900 High Throughput nucleic acid synthesizer (manufactured by Applied Biosystems). After completion of the synthesis, desorption from a solid phase, removal of a protecting group, purification of the object product and the like are performed. It is desirable to obtain an antisense oligonucleotide having purity of 90% or more, preferably 95% or more, by purification. As an enzymatic transcription method for producing the antisense oligonucleotide of the present invention, a method using a plasmid or DNA having the object nucleotide sequence as a template, and including transcription using phage RNA polymerase, for example, T7, T3, or SP6RNA polymerase, can be mentioned.

The antisense oligonucleotide of the present invention can be introduced into a cell by using a carrier for transfection, preferably a cationic carrier such as cationic liposome and the like. Also, it can be directly introduced into a cell by a calcium phosphate method, an electroporation method, a microinjection method and the like.

It is also possible to induce suppression of the expression of the target gene by forming a double strand of the antisense oligonucleotide of the present invention and the complementary oligonucleic acid, and introducing the double strand nucleic acid into the cell (patent document WO 2005/113571). In this case, the position of modification of the double strand nucleic acid with a ligand is preferably the 5′-terminal or 3′-terminal of the complementary oligonucleic acid.

### 2. Antisense Oligonucleotide Having Expression Suppressive Activity on β2GPI

The antisense oligonucleotide of the present invention composed of a base sequence complementary to a part of the base sequence of β2GPI gene can be designed based on, for example, a cDNA base sequence (SEQ ID NO: 1) of the full length mRNA of human β2GPI registered as Genbank Accession No. NM_000042 or genomic sequence. The cDNA of the full length mRNA of human β2GPI is registered as, for example, Genbank Accession No. NM_000042, and a genomic sequence containing mRNA precursor of human β2GPI is registered as, for example, Genbank Accession No. NC_000017.11.

Of the nucleic acids composed of a base sequence complementary to a part of the target base sequence of the β2GPI gene, the antisense oligonucleotide having an expression suppress activity against β2GPI includes, for example, antisense oligonucleotides constituted of the base sequences selected from the groups described in Table 1-Table 4. The length of the antisense oligonucleotides is 8-80 bases, and 8-30 bases are preferable. For example, 8-20 bases, 10-20 bases, 13-20 bases, 13-16 bases, 13 bases, 14 bases, 15 bases, 16 bases, 17 bases, 18 bases, 19 bases, 20 bases.

The expression of β2GPI can be suppressed by introducing these antisense oligonucleotides into a cell. For example, the antisense oligonucleotide of the present invention introduced into a cell at a concentration of several nM-several μM can suppress expression of β2GPI when cultured for 24 hr or more, for example, 48 hr.

Furthermore, the evaluation of the expression suppressive activity of the antisense oligonucleotide of the present invention on β2GPI can be performed by introducing the antisense oligonucleotide by using a cationic liposome and the like, or the antisense oligonucleotide as it is, or the antisense oligonucleotide bound to a certain ligand, into human cell line and the like, culturing same for a given period, and quantifying the expression level of β2GPI mRNA in the human cell line.

The antisense oligonucleotide of the present invention may contain a ligand. The ligand may directly modify the 5′terminal, 3′-terminal and/or inside of sequence of the antisense oligonucleotide of the present invention.

While the ligand contained in the antisense oligonucleotide of the present invention may be a molecule having affinity for a biological molecule, for example, lipids such as cholesterol, fatty acid, tocopherol, retinoid and the like, saccharides such as N-acetylgalactosamine (GalNAc), galactose (Gal), mannose (Man) and the like, antibodies such as full antibody, Fab, scFv, VHH and the like, proteins such as low-density lipoprotein (LDL), human serum albumin and the like, peptides such as RGD, NGR, R9, CPP and the like, small molecules such as folic acid and the like, synthesis polymers such as synthetic polyamino acid and the like, nucleic acid aptamers and the like can be mentioned, but it is not limited to these and these can also be used in combination.

Examples of the method for adding a ligand to the antisense oligonucleotide of the present invention include, but are not limited to, reacting a modifier, capable of reaction on the solid phase, during an elongation reaction on the solid phase, whereby the 5-′terminal, 3′-terminal and/or inside of sequence can be modified. In addition, a conjugate nucleic acid can also be obtained by synthesizing and purifying in advance a nucleic acid introduced with a functional group such as amino group, mercapto group, azido group or triple bond and the like, and reacting same with a modifier.

### 3. Pharmaceutical Composition of the Present Invention

The present invention relates to a pharmaceutical composition containing the antisense oligonucleotide of the present invention as an active ingredient (to be also referred to as the pharmaceutical composition of the present invention in the present specification).

The pharmaceutical composition can further contain a carrier effective for intracellular transfer of the antisense oligonucleotide of the present invention. The pharmaceutical composition of the present invention can be used as a therapeutic or prophylactic agent for autoimmune diseases such as APS and SLE, and thrombosis in hemodialysis and the like.

Examples of the carrier effective for intracellular transfer of the antisense oligonucleotide of the present invention include cationic carriers. Examples of the cationic carrier include a cationic liposome, a cationic polymer and the like. As a carrier effective for intracellular transfer of the antisense oligonucleotide, a carrier utilizing a virus envelope may also be used. As a cationic liposome, a liposome containing 2-O-(2-diethylaminoethyl)carbamoyl-1,3-O-dioleoylglycerol (hereinafter to be also referred to as liposome A), oligofectamine (Invitrogen), Lipofectin (Invitrogen), lipofectamine (Invitrogen), lipofectamine2000 (Invitrogen), DMRIE-C (Invitrogen), GeneSilencer (Gene Therapy Systems), TransMessenger (QIAGEN), TransIT TKO (Mirus) and the like are preferably used. As a cationic polymer, JetSI (Qbiogene Inc.), Jet-PEI (polyethyleneimine; Qbiogene Inc.) and the like are preferably used. As a carrier utilizing a virus envelope, GenomeOne (HVJ-E liposome; ISHIHARA SANGYO KAISHA, LTD.) and the like are preferably used.

The pharmaceutical composition of the present invention comprising the antisense oligonucleotide of the present invention and the above-mentioned carrier can be prepared by a method known to those of ordinary skill in the art. For example, it can be prepared by mixing a carrier dispersion liquid and an antisense oligonucleotide solution at suitable concentrations. When a cationic carrier is used, it can be prepared easily by mixing in an aqueous solution by a conventional method, since antisense oligonucleotide has a negative electric charge in aqueous solutions. Examples of the aqueous solvent used for the preparation of the composition include electrolytic solutions such as water for injection, distilled water for injection, saline and the like, sugar solutions such as glucose solution, maltose solution and the like, and the like. The conditions such as pH and temperature and the like for preparation of the composition can be appropriately selected by those of ordinary skill in the art. In the case of liposome A, for example, the pharmaceutical composition can be prepared by gradually adding antisense oligonucleotide in 10% aqueous maltose solution to 16 mg/ml liposome dispersion in 10% aqueous maltose solution at pH 7.4, 25° C. with stirring.

Where necessary, the composition can also be formed as a uniform composition by a dispersion treatment using an ultrasonic dispersion apparatus, a high-pressure emulsion apparatus and the like. Since the method and conditions optimal for the preparation of a composition comprising a carrier and an antisense oligonucleotide depend on the carrier to be used, those of ordinary skill in the art can select an optimal method for the carrier to be used irrespective of the above-mentioned methods.

As the pharmaceutical composition of the present invention, a liposome constituted of a composite particle comprising an antisense oligonucleotide and a lead particle as constituent components, and a lipid membrane covering the composite particle, wherein a liquid containing a polar organic solvent at a concentration at which the constituent components of the lipid membrane can be dispersed and the composite particles can be dispersed is present in a liquid containing the polar organic solvent in which the constituent components of the lipid membrane can be dissolved, can also be used preferably, though the pharmaceutical composition is not limited thereto. Examples of the lead particle include a lipid assembly, a liposome, an emulsion particle, a polymer, a metal colloid, a fine particle preparation and the like, and a liposome is preferably used. The lead particle in the present invention may contain a complex of a combination of two or more from a lipid assembly, a liposome, an emulsion particle, a polymer, a metal colloid, a fine particle preparation and the like as a constituent component, or a complex of a combination of a lipid assembly, a liposome, an emulsion particle, a polymer, a metal colloid, a fine particle preparation and the like and other compound (e.g., sugar, lipid, inorganic compound etc.) as a constituent component.

Examples of the lipid membrane covering the composite particle include those comprising, for example, neutral lipid and polyethylene glycol-phosphatidyl ethanolamine and the like as the constituent components.

The liposome can be prepared according to, for example, the method described in WO 2006/080118 and the like.

A suitable mixing ratio of the antisense oligonucleotide and the carrier comprised in the pharmaceutical composition of the present invention is 1-200 parts by weight of a carrier per 1 part by weight of antisense oligonucleotide. It is preferably 2.5-100 parts by weight, further preferably 10-parts by weight, of a carrier per 1 part by weight of antisense oligonucleotide.

The pharmaceutical composition of the present invention may also comprise a pharmaceutically acceptable carrier, a diluent and the like besides the above-mentioned carrier. A pharmaceutically acceptable carrier, a diluent and the like are essentially chemically-inactive and harmless compositions, and do not at all influence the biological activity of the pharmaceutical composition of the present invention. Examples of the carrier and diluent include, but are not limited to, a salt solution, a sugar solution, a glycerol solution, ethanol and the like.

The pharmaceutical composition of the present invention can be preferably used for the treatment or prophylaxis of diseases relating to the expression of β2GPI, particularly, disorders mediated by an anti-β2GPI antibody. In the present specification, the disorders mediated by an anti-β2GPI antibody refer to autoimmune diseases such as antiphospholipid antibody syndrome (APS) and SLE, and thrombosis in hemodialysis. Therefore, the pharmaceutical composition of the present invention can be used as a therapeutic agent or a prophylaxis agent for autoimmune diseases such as APS and SLE, and thrombosis in hemodialysis.

The pharmaceutical composition of the present invention comprises the complex in an amount effective for the treatment or prevention of diseases and is provided in a form permitting appropriate administration to patients. The formulation of the pharmaceutical composition of the present invention may be, for example, a liquid such as injection, eye drop, inhalation and the like, for example, an external preparation such as ointment, lotion and the like, and the like.

In the case of a liquid, the concentration range of the pharmaceutical composition of the present invention is generally 0.001-25% (w/v), preferably 0.01-5% (w/v), more preferably 0.1-2% (w/v). The pharmaceutical composition of the present invention may comprise an adequate amount of any pharmaceutically acceptable additive, for example, an emulsion adjuvant, a stabilizer, an isotonicifier, a pH adjuster and the like. Any pharmaceutically acceptable additive can be added in a suitable step before or after dispersion of the complex.

The pharmaceutical composition of the present invention can also be provided as a freeze-dried preparation. A freeze-dried preparation can be prepared by a dispersion treatment of an antisense oligonucleotide and a carrier, followed by a freeze-drying treatment. A freeze-drying treatment can be performed by a conventional method. For example, a given amount of a complex solution after the above-mentioned dispersion treatment is dispensed in a vial container under sterile conditions, predried for about 2 hr under the condition of about −40° C. to −20° C., primarily predried at about 0-10° C. under reduced pressure, then secondarily dried at about 15-25° C. under reduced pressure to perform freeze-drying. Then, for example, the inside of the vial is substituted with a nitrogen gas and a cap is provided, whereby a freeze-dried preparation of the pharmaceutical composition of the present invention can be obtained.

When the pharmaceutical composition of the present invention is provided as a freeze-dried preparation, the pharmaceutical composition of the present invention can be used by redissolving by the addition of any suitable solution. Examples of the solution include electrolytic solutions such as water for injection, saline and the like, glucose solution, other general infusions and the like. While the liquid volume of this solution varies depending on the use and the like and is not particularly limited, it is preferably a 0.5- to 2-fold amount of the liquid volume before freeze-drying, or 500 ml or less.

The pharmaceutical composition of the present invention can be administered to animals including human by, for example, intravenous administration, intraarterial administration, oral administration, tissue administration, transdermal administration, transmucosal administration or rectal administration, and is preferably administered by an appropriate method according to the symptom of the patient. Particularly, intravenous administration, transdermal administration, and transmucosal administration are preferably used. In addition, topical administration such as topical administration to a cancer site and the like can also be employed. Examples of the dosage form suitable for these administration methods include various injections, oral preparations, drip infusions, absorbents, eye drops, ointments, lotions, suppositories and the like.

While the dose of the pharmaceutical composition of the present invention is desirably determined in consideration of drug, dosage form, condition of patient such as age, body weight and the like, administration route, nature and severity of the disease and the like, it is generally 0.1 mg-10 g/day, preferably 1 mg-500 mg/day, for an adult in the mass of the antisense oligonucleotide. In some cases, a dose below these levels may be sufficient, or a dose above these levels may be conversely required. The pharmaceutical composition can be administered one to several times per day, or can be administered at one to several day intervals.

### 4. Treatment Method

The present invention further provides a method of treating diseases related to the expression of β2GPI, particularly, disorders mediated by an anti-β2GPI antibody, comprising a step of administering a therapeutically effective amount of the antisense oligonucleotide of the present invention or the pharmaceutical composition of the present invention to a human in need of such treatment (treatment method of the present invention).

The treatment method of the present invention is preferably a method of treating autoimmune diseases or thrombosis, which is characterized by administering a therapeutically effective amount of the antisense oligonucleotide of the present invention or the pharmaceutical composition of the present invention to a human in need of such treatment. Other steps and conditions are not limited in any manner.

In the treatment method of the present invention, for example, the administration method, dose, preparation method and the like of the aforementioned pharmaceutical composition of the present invention can be used.

The present invention is explained in the following by referring to Examples, which are not to be construed as limitative.

### Example 1

Measurement of Knockdown Activity of β2GPI mRNA

Huh7 cell (obtained from National Institutes of Biomedical Innovation, Health and Nutrition JCRB Cell Bank), which is a cell line derived from human liver cancer, were seeded to a 96-well culture plate at 5,000 cells/80 μL/well. As a medium, MEM medium (manufactured by Life technologies, catalog No. 11095-098) containing 10% fetal bovine serum (FBS) was used. As the antisense oligonucleotides, those described in Table 2-Table 4 were synthesized by GeneDesign, Inc. or Hokkaido System Science CO., Ltd and used. In the antisense oligonucleotides of Table 2, lower case letters indicate DNA and capital letters indicate 2′-O-methylated RNA. In the antisense oligonucleotides of Table 3 and Table 4, lower case letters indicate DNA, capital letters indicate LNA, and mC shows 5-methylcytosine LNA. In all antisense oligonucleotides of Table 2-Table 4, the phosphoric acid diester bond in each nucleotide is substituted by a phosphorothioate bond. The antisense oligonucleotide and Lipofectamine LTX & Plus reagent (manufactured by Life technologies, catalog No.: 15338) were diluted with Opti-MEM medium (manufactured by Life technologies, catalog No. 11058-021), and 20 μL of each antisense oligonucleotide/Lipofectamine mixture was added to 96-well culture plate to the final concentration of antisense oligonucleotide of 100 nM, and the mixture was cultured under the conditions 37° C., 5% CO₂ for 24 hr. The cells were washed with PBS (phosphate buffered saline), and cDNA was synthesized from each plate by using Cells-to-Ct kit (manufactured by Applied Biosystems, catalog No.: AM1728) and according to the method described in the manual attached to the product. The cDNA (5 μL) was added to MicroAmp Optical 96-well plate (manufactured by Applied Biosystems, catalog No. 4326659), and μL of TaqMan Gene Expression Master Mix (manufactured by Applied Biosystems, catalog No. 4369016), 3 μL of UltraPure Distilled Water (manufactured by Life technologies, catalog No.: 10977-015), 1 μL of human β2GPI probe, and 1 μL of human GAPDH probe were further added. The real-time PCR of human β2GPI gene and human GAPDH (D-glyceraldehyde-3-phosphate dehydrogenase) was performed by using the ABI7900 HT real-time PCR system. GAPDH is a constitutively expressed gene and was measured as the internal control, and the β2GPI expression level was normalized. The β2GPI mRNA relative expression level when each antisense oligonucleotide was introduced, was calculated relative to the β2GPI mRNA amount when Huh7 cells were treated with a transfection reagent alone without addition of antisense oligonucleotide as 1.0. This experiment was performed 2 times and the mean of the β2GPI mRNA relative expression level is shown in Tables 2 to 4.

TABLE 2-1

| SEQ | antinsense | SEQ | antinsense | β2GPI relative |
|---|---|---|---|---|
| ID | base | ID | oligonucleotide | expression |
| NO: | sequence | NO: | (5′→3′) | level |
| 2 | gagcactggagaa | 400 | GAgcactggaGAA | 0.570 |
| 3 | tgagcactggaga | 401 | UGagcactggAGA | 0.652 |
| 4 | agatgagcactgg | 402 | AGatgagcacUGG | 0.652 |
| 5 | agcaacatggcag | 403 | AGcaacatggCAG | 0.660 |
| 6 | tagcaacatggca | 404 | UAgcaacatgGCA | 0.697 |
| 7 | atagcaacatggc | 405 | AUagcaacatGGC | 0.648 |
| 12 | gtcctgcaatagc | 406 | GUcctgcaatAGC | 0.528 |
| 14 | gtccgtcctgcaa | 407 | GUccgtcctgCAA | 0.388 |
| 15 | ggtccgtcctgca | 408 | GGtccgtcctGCA | 0.447 |
| 16 | aggtccgtcctgc | 409 | AGgtccgtccUGC | 0.517 |
| 17 | caggtccgtcctg | 410 | CAggtccgtcCUG | 0.627 |
| 18 | tgggacaggtccg | 411 | UGggacaggtCCG | 0.583 |
| 19 | ttgggacaggtcc | 412 | UUgggacaggUCC | 0.538 |
| 20 | ggcttgggacagg | 413 | GGcttgggacAGG | 0.691 |
| 21 | tggcttgggacag | 414 | UGgcttgggaCAG | 0.555 |
| 22 | ctggcttgggaca | 415 | CUggcttgggACA | 0.412 |
| 23 | tctggcttgggac | 416 | UCtggcttggGAC | 0.583 |
| 24 | catctggcttggg | 417 | CAtctggcttGGG | 0.663 |
| 25 | tcatctggcttgg | 418 | UCatctggctUGG | 0.373 |
| 26 | atcatctggcttg | 419 | AUcatctggcUUG | 0.537 |
| 27 | aatcatctggctt | 420 | AAtcatctggCUU | 0.621 |
| 28 | aaatcatctggct | 421 | AAatcatctgGCU | 0.681 |
| 29 | tcctggctcatag | 422 | UCctggctcaUAG | 0.601 |
| 30 | ctcctggctcata | 423 | CUcctggctcAUA | 0.537 |
| 31 | tctcctggctcat | 424 | UCtcctggctCAU | 0.402 |
| 32 | ccggcttgcagga | 425 | CCggcttgcaGGA | 0.567 |
| 33 | cccggcttgcagg | 426 | CCcggcttgcAGG | 0.581 |
| 34 | gcccggcttgcag | 427 | GCccggcttgCAG | 0.477 |
| 35 | agcccggcttgca | 428 | AGcccggcttGCA | 0.325 |
| 36 | tagcccggcttgc | 429 | UAgcccggctUGC | 0.310 |
| 37 | atagcccggcttg | 430 | AUagcccggcUUG | 0.402 |
| 38 | catagcccggctt | 431 | CAtagccoggCUU | 0.583 |
| 39 | acacatagcccgg | 432 | ACacatagccCGG | 0.629 |
| 40 | gacacatagcccg | 433 | GAcacatagcCCG | 0.687 |
| 41 | tgtgagagggcag | 434 | UGtgagagggCAG | 0.652 |
| 42 | ttgatgggccaca | 435 | UUgatgggccACA | 0.572 |
| 43 | gttgatgggccac | 436 | GUtgatgggcCAC | 0.606 |
| 44 | tgttgatgggcca | 437 | UGttgatgggCCA | 0.479 |
| 45 | gtgttgatgggcc | 438 | GUgttgatggGCC | 0.535 |
| 46 | ggacatactctgg | 439 | GGacatactoUGG | 0.505 |
| 47 | aggacatactctg | 440 | AGgacatactCUG | 0.487 |
| 50 | gctccattttcta | 441 | GCtccattttCUA | 0.606 |
| 54 | qttgqqatattca | 442 | GUtgggatatUCA | 0.663 |
| 55 | gcacttggcagaa | 443 | GCacttggcaGAA | 0.499 |
| 56 | tgcacttggcaga | 444 | UGcacttggcAGA | 0.517 |
| 57 | gtgcacttggcag | 445 | GUgcacttggCAG | 0.281 |
| 58 | agtgcacttggca | 446 | AGtgcacttgGCA | 0.482 |
| 59 | cagtgcacttggc | 447 | CAgtgcacttGGC | 0.480 |
| 60 | ctcagtgcacttg | 448 | CUcagtgcacUUG | 0.456 |
| 61 | cctcagtgcactt | 449 | CCtcagtgcaCUU | 0.387 |
| 62 | tcctcagtgcact | 450 | UCctcagtgcACU | 0.226 |
| 63 | ttcctcagtgcac | 451 | UUcctcagtgCAC | 0.334 |
| 64 | cttcctcagtgca | 452 | CUtcctcagtGCA | 0.364 |
| 65 | ccttcctcagtgc | 453 | CCttcctcagUGC | 0.415 |
| 66 | tccttcctcagtg | 454 | UCcttcctcaGUG | 0.425 |
| 67 | gctgatggcttat | 455 | GCtgatggctUAU | 0.685 |
| 68 | agctgatggctta | 456 | AGctgatggcUUA | 0.275 |
| 69 | cagctgatggctt | 457 | CAgctgatggCUU | 0.225 |
| 70 | ccagctgatggct | 458 | CCagctgatgGCU | 0.421 |
| 71 | tccagctgatggc | 459 | UCcagctgatGGC | 0.217 |
| 72 | ttccagctgatgg | 460 | UUccagctgaUGG | 0.454 |
| 73 | gtttccagctgat | 461 | GUttccagctGAU | 0.535 |
| 74 | tgtttccagctga | 462 | UGtttccagcUGA | 0.249 |
| 75 | ttgtttccagctg | 463 | UUgtttccagCUG | 0.302 |
| 76 | aactgctgtgtcc | 464 | AActgctgtgUCC | 0.546 |
| 77 | aaaaactgctgtg | 465 | AAaaactgctGUG | 0.475 |
| 78 | tggcaaacattca | 466 | UGgcaaacatUCA | 0.581 |
| 79 | gtggcaaacattc | 467 | GUggcaaacaUUC | 0.660 |
| 80 | gttgtggcaaaca | 468 | GUtgtggcaaACA | 0.654 |
| 81 | tgttgtggcaaac | 469 | UGttgtggcaAAC | 0.676 |
| 82 | gcatgttgtggca | 470 | GCatgttgtgGCA | 0.408 |
| 83 | cgcatgttgtggc | 471 | CGcatgttgtGGC | 0.243 |
| 84 | tcgcatgttgtgg | 472 | UCgcatgttgUGG | 0.613 |
| 85 | atcgcatgttgtg | 473 | AUcgcatgttGUG | 0.685 |
| 86 | acatcgcatgttg | 474 | ACatcgcatgUUG | 0.683 |
| 87 | ttccaaacatcgc | 475 | UUccaaacatCGC | 0.576 |
| 88 | agtccaatttcca | 476 | AGtccaatttCCA | 0.626 |
| 89 | ccctgcattctgg | 477 | CCctgcattcUGG | 0.583 |
| 90 | tccctgcattctg | 478 | UCcctgcattCUG | 0.548 |
| 91 | ttccctgcattct | 479 | UUccctgcatUCU | 0.683 |
| 92 | attgtctggtctt | 480 | AUtgtctggtCUU | 0.562 |
| 93 | cattgtctggtct | 481 | CAttgtctggUCU | 0.356 |
| 94 | ccattgtctggtc | 482 | CCattgtctgGUC | 0.253 |
| 95 | tccattgtctggt | 483 | UCcattgtctGGU | 0.345 |
| 96 | atccattgtctgg | 484 | AUccattgtcUGG | 0.334 |
| 97 | aatccattgtctg | 485 | AAtccattgtCUG | 0.669 |
| 98 | gcaggatagttca | 486 | GCaggatagtUCA | 0.550 |
| 99 | ggttttgcaggat | 487 | GGttttgcagGAU | 0.562 |
| 100 | tggttttgcagga | 488 | UGgttttgcaGGA | 0.537 |
| 101 | ggctttatccttg | 489 | GGctttatccUUG | 0.610 |
| 102 | gtggctttatcct | 490 | GUggctttatCCU | 0.672 |
| 106 | catcatggcagcc | 491 | CAtcatggcaGCC | 0.688 |
| 107 | ccatcatggcagc | 492 | CCatcatggcAGC | 0.427 |
| 108 | tccatcatggcag | 493 | UCcatcatggCAG | 0.691 |
| 109 | atccatcatggca | 494 | AUccatcatgGCA | 0.614 |
| 110 | tatccatcatggc | 495 | UAtccatcatGGC | 0.407 |
| 111 | atatccatcatgg | 496 | AUatccatcaUGG | 0.607 |
| 112 | cccagtttggtac | 497 | CCcagtttggUAC | 0.378 |
| 113 | tcccagtttggta | 498 | UCccagtttgGUA | 0.637 |
| 114 | ttcccagtttggt | 499 | UUcccagtttGGU | 0.512 |
| 115 | tttcccagtttgg | 500 | UUtcccagttUGG | 0.632 |
| 116 | gtttcccagtttg | 501 | GUttoccagtUUG | 0.518 |
| 117 | cagtttcccagtt | 502 | CAgtttoccaGUU | 0.581 |
| 118 | ccagtttcccagt | 503 | CCagtttoccAGU | 0.548 |
| 119 | accagtttcccag | 504 | ACcagtttccCAG | 0.631 |
| 120 | gaccagtttccca | 505 | GAccagtttcCCA | 0.313 |
| 121 | agaccagtttccc | 506 | AGaccagtttCCC | 0.405 |
| 122 | gcagaccagtttc | 507 | GCagaccagtUUC | 0.694 |
| 123 | ggcagaccagttt | 508 | GGcagaccagUUU | 0.399 |
| 124 | tggcagaccagtt | 509 | UGgcagaccaGUU | 0.219 |
| 125 | atggcagaccagt | 510 | AUggcagaccAGU | 0.518 |
| 126 | catggcagaccag | 511 | CAtggcagacCAG | 0.426 |
| 127 | gcatggcagacca | 512 | GCatggcagaCCA | 0.374 |
| 128 | ggcatggcagacc | 513 | GGcatggcagACC | 0.246 |
| 129 | tggcatggcagac | 514 | UGgcatggcaGAC | 0.203 |
| 130 | ttggcatggcaga | 515 | UUggcatggcAGA | 0.479 |
| 131 | cttggcatggcag | 516 | CUtggcatggCAG | 0.305 |
| 132 | acttggcatggca | 517 | ACttggcatgGCA | 0.356 |
| 133 | aacttggcatggc | 518 | AActtggcatGGC | 0.221 |
| 134 | caacttggcatgg | 519 | CAacttggcaUGG | 0.465 |
| 135 | acaacttggcatg | 520 | ACaacttggcAUG | 0.679 |
| 136 | gctttacaacttg | 521 | GCtttacaacUUG | 0.598 |
| 137 | tcacaggtacttt | 522 | UCacaggtacUUU | 0.666 |
| 138 | acaccacagtggc | 523 | ACaccacagtGGC | 0.158 |
| 139 | gtacaccacagtg | 524 | GUacaccacaGUG | 0.478 |
| 140 | ggtacaccacagt | 525 | GGtacaccacAGU | 0.267 |
| 141 | tggtacaccacag | 526 | UGgtacaccaCAG | 0.235 |
| 142 | ttggtacaccaca | 527 | UUggtacaccACA | 0.370 |
| 143 | cttggtacaccac | 528 | CUtggtacacCAC | 0.607 |
| 144 | ccttggtacacca | 529 | CCttggtacaCCA | 0.271 |
| 145 | tccttggtacacc | 530 | UCcttggtacACC | 0.544 |
| 146 | ctccttggtacac | 531 | CUccttggtaCAC | 0.325 |
| 147 | tctccttggtaca | 532 | UCtocttggtACA | 0.496 |
| 148 | ctctccttggtac | 533 | CUctccttggUAC | 0.340 |
| 149 | tctctccttggta | 534 | UCtctccttgGUA | 0.624 |
| 150 | ctctctccttggt | 535 | CUctctccttGGU | 0.397 |
| 151 | totctotccttgg | 536 | UCtctctcctUGG | 0.309 |
| 152 | ctctctctccttg | 537 | CUctctctccUUG | 0.372 |
| 153 | actctctctcctt | 538 | ACtctctctcCUU | 0.526 |
| 154 | tactctctctcct | 539 | UActctctctCCU | 0.489 |
| 155 | ttactctctctcc | 540 | UUactctctcUCC | 0.604 |
| 156 | gcattccattctt | 541 | GCattccattCUU | 0.564 |
| 157 | agcattccattct | 542 | AGcattccatUCU | 0.342 |
| 158 | agctacacttctt | 543 | AGctacacttCUU | 0.507 |
| 159 | tagctacacttct | 544 | UAgctacactUCU | 0.647 |
| 160 | ttgaagcatttgg | 545 | UUgaagcattUGG | 0.697 |
| 161 | gttccttgaagca | 546 | GUtccttgaaGCA | 0.332 |
| 162 | tgttccttgaagc | 547 | UGttccttgaAGC | 0.324 |
| 163 | gtgttccttgaag | 548 | GUgttccttgAAG | 0.496 |
| 164 | tgtgttccttgaa | 549 | UGtgttccttGAA | 0.511 |
| 165 | ctgtgttccttga | 550 | CUgtgttcctUGA | 0.304 |
| 166 | actgtgttccttg | 551 | ACtgtgttccUUG | 0.353 |
| 167 | aactgtgttcctt | 552 | AActgtgttcCUU | 0.449 |
| 168 | gaactgtgttcct | 553 | GAactgtgttCCU | 0.262 |
| 169 | agaactgtgttcc | 554 | AGaactgtgtUCC | 0.416 |
| 170 | gagaactgtgttc | 555 | GAgaactgtgUUC | 0.588 |
| 171 | agagaactgtgtt | 556 | AGagaactgtGUU | 0.623 |
| 172 | cagagaactgtgt | 557 | CAgagaactgUGU | 0.569 |
| 173 | ccagagaactgtg | 558 | CCagagaactGUG | 0.428 |
| 174 | gccagagaactgt | 559 | GCcagagaacUGU | 0.457 |
| 175 | agccagagaactg | 560 | AGccagagaaCUG | 0.314 |
| 176 | aagccagagaact | 561 | AAgccagagaACU | 0.611 |
| 177 | gcatcagttttcc | 562 | GCatcagtttUCC | 0.686 |
| 178 | tgcatcagttttc | 563 | UGcatcagttUUC | 0.693 |
| 179 | gatgcatcagttt | 564 | GAtgcatcagUUU | 0.534 |
| 180 | ggatgcatcagtt | 565 | GGatgcatcaGUU | 0.134 |
| 181 | cggatgcatcagt | 566 | CGgatgcatcAGU | 0.188 |
| 182 | tcggatgcatcag | 567 | UCggatgcatCAG | 0.227 |
| 183 | atcggatgcatca | 568 | AUcggatgcaUCA | 0.386 |
| 184 | catcggatgcatc | 569 | CAtcggatgcAUC | 0.497 |
| 185 | acatcggatgcat | 570 | ACatcggatgCAU | 0.528 |
| 186 | tacatcggatgca | 571 | UAcatcggatGCA | 0.594 |
| 187 | ttacatcggatgc | 572 | UUacatcggaUGC | 0.594 |
| 188 | gctttacatcgga | 573 | GCtttacatcGGA | 0.626 |
| 189 | ggctttacatcgg | 574 | GGctttacatCGG | 0.359 |
| 190 | tggctttacatcg | 575 | UGgctttacaUCG | 0.411 |
| 191 | tgtggaatctgaa | 576 | UGtggaatctGAA | 0.671 |
| 192 | agtgtgacatttt | 577 | AGtgtgacatUUU | 0.428 |
| 193 | aagtgtgacattt | 578 | AAgtgtgacaUUU | 0.440 |
| 194 | ccttggatgaaca | 579 | CCttggatgaACA | 0.690 |
| 195 | tccttggatgaac | 580 | UCcttggatgAAC | 0.669 |
| 196 | gttccttggatga | 581 | GUtccttggaUGA | 0.558 |
| 197 | ggttccttggatg | 582 | GGttccttggAUG | 0.432 |
| 198 | aggttccttggat | 583 | AGgttcottgGAU | 0.354 |
| 199 | taggttccttgga | 584 | UAggttccttGGA | 0.278 |
| 200 | ttaggttccttgg | 585 | UUaggttcctUGG | 0.278 |

TABLE 3-1

| SEQ | antinsense | SEQ | antinsense | β2GPI relative |
|---|---|---|---|---|
| ID | base | ID | oligonucleotide | expression |
| NO: | sequence | NO: | (5′→3′) | level |
| 6 | tagcaacatggca | 586 | TAgcaacatgGmCA | 0.033 |
| 7 | atagcaacatggc | 587 | ATagcaacatGGmC | 0.067 |
| 8 | caatagcaacatg | 588 | mCAatagcaacATG | 0.065 |
| 9 | gcaatagcaacat | 589 | GmCaatagcaamCAT | 0.220 |
| 10 | cctgcaatagcaa | 590 | mCmCtgcaatagmCAA | 0.093 |
| 11 | tcctgcaatagca | 591 | TmCctgcaataGmCA | 0.043 |
| 12 | gtcctgcaatagc | 592 | GTcctgcaatAGmC | 0.025 |
| 13 | tccgtcctgcaat | 593 | TmCcgtcctgcAAT | 0.082 |
| 14 | gtccgtcctgcaa | 594 | GTccgtcctgmCAA | 0.161 |
| 15 | ggtccgtcctgca | 595 | GGtccgtcctGmCA | 0.141 |
| 22 | ctggcttgggaca | 596 | mCTggcttgggAmCA | 0.169 |
| 25 | tcatctggcttgg | 597 | TmCatctggctTGG | 0.134 |
| 31 | tctcctggctcat | 598 | TmCtcctggctmCAT | 0.594 |
| 35 | agcccggcttgca | 599 | AGcccggcttGmCA | 0.295 |
| 36 | tagcccggcttgc | 600 | TAgcccggctTGmC | 0.231 |
| 37 | atagcccggcttg | 601 | ATagcccggcTTG | 0.357 |
| 45 | gtgttgatgggcc | 602 | GTgttgatggGmCmC | 0.058 |
| 46 | ggacatactctgg | 603 | GGacatactcTGG | 0.009 |
| 48 | aaggacatactct | 604 | AAggacatacTmCT | 0.020 |
| 49 | aaaggacatactc | 605 | AAaggacatamCTmC | 0.012 |
| 51 | gggatattcaaaa | 606 | GGgatattcaAAA | 0.294 |
| 52 | tgggatattcaaa | 607 | TGggatattcAAA | 0.104 |
| 53 | ttgggatattcaa | 608 | TTgggatattmCAA | 0.025 |
| 57 | gtgcacttggcag | 609 | GTgcacttggmCAG | 0.212 |
| 61 | cctcagtgcactt | 610 | mCmCtcagtgcamCTT | 0.201 |
| 62 | tcctcagtgcact | 611 | TmCctcagtgcAmCT | 0.122 |
| 65 | ccttcctcagtgc | 612 | mCmCttcctcagTGmC | 0.369 |
| 68 | agctgatggctta | 613 | AGctgatggcTTA | 0.193 |
| 69 | cagctgatggctt | 614 | mCAgctgatggmCTT | 0.448 |
| 70 | ccagctgatggct | 615 | mCmCagctgatgGmCT | 0.148 |
| 71 | tccagctgatggc | 616 | TmCcagctgatGGmC | 0.289 |
| 74 | tgtttccagctga | 617 | TGtttccagcTGA | 0.084 |
| 75 | ttgtttccagctg | 618 | TTgtttccagmCTG | 0.042 |
| 82 | gcatgttgtggca | 619 | GmCatgttgtgGmCA | 0.218 |
| 83 | cgcatgttgtggc | 620 | mCGcatgttgtGGmC | 0.053 |
| 93 | cattgtctggtct | 621 | mCAttgtctggTmCT | 0.217 |
| 94 | ccattgtctggtc | 622 | mCmCattgtctgGTmC | 0.324 |
| 95 | tccattgtctggt | 623 | TmCcattgtctGGT | 0.422 |
| 96 | atccattgtctgg | 624 | ATccattgtcTGG | 0.079 |
| 103 | atgtggctttatc | 625 | ATgtggctttATmC | 0.074 |
| 104 | aatgtggctttat | 626 | AAtgtggcttTAT | 0.111 |
| 105 | ccaaatgtggctt | 627 | mCmCaaatgtggmCTT | 0.019 |
| 107 | ccatcatggcagc | 628 | mCmCatcatggcAGmC | 0.129 |
| 110 | tatccatcatggc | 629 | TAtccatcatGGmC | 0.204 |
| 112 | cccagtttggtac | 630 | mCmCcagtttggTAmC | 0.210 |
| 120 | gaccagtttccca | 631 | GAccagtttcmCmCA | 0.414 |
| 121 | agaccagtttccc | 632 | AGaccagtttmCmCmC | 0.436 |
| 123 | ggcagaccagttt | 633 | GGcagaccagTTT | 0.138 |
| 124 | tggcagaccagtt | 634 | TGgcagaccaGTT | 0.187 |
| 126 | catggcagaccag | 635 | mCAtggcagacmCAG | 0.134 |
| 127 | gcatggcagacca | 636 | GmCatggcagamCmCA | 0.091 |
| 128 | ggcatggcagacc | 637 | GGcatggcagAmCmC | 0.163 |
| 129 | tggcatggcagac | 638 | TGgcatggcaGAmC | 0.265 |
| 131 | cttggcatggcag | 639 | mCTtggcatggmCAG | 0.134 |
| 132 | acttggcatggca | 640 | AmCttggcatgGmCA | 0.086 |
| 133 | aacttggcatggc | 641 | AActtggcatGGmC | 0.061 |
| 138 | acaccacagtggc | 642 | AmCaccacagtGGmC | 0.501 |
| 140 | ggtacaccacagt | 643 | GGtacaccacAGT | 0.137 |
| 144 | ccttggtacacca | 644 | mCmCttggtacamCmCA | 0.265 |
| 145 | tccttggtacacc | 645 | TmCcttggtacAmCmC | 0.232 |
| 146 | ctccttggtacac | 646 | mCTccttggtamCAmC | 0.493 |
| 148 | ctctccttggtac | 647 | mCTctccttggTAmC | 0.119 |
| 150 | ctctctccttggt | 648 | mCTctctccttGGT | 0.250 |
| 151 | tctctctccttgg | 649 | TmCtctctcctTGG | 0.196 |
| 152 | ctctctctccttg | 650 | mCTctctctccTTG | 0.456 |
| 157 | agcattccattct | 651 | AGcattccatTmCT | 0.283 |
| 161 | gttccttgaagca | 652 | GTtccttgaaGmCA | 0.067 |
| 162 | tgttccttgaagc | 653 | TGttccttgaAGmC | 0.112 |
| 164 | tgtgttccttgaa | 654 | TGtgttccttGAA | 0.271 |
| 165 | ctgtgttccttga | 655 | mCTgtgttcctTGA | 0.148 |
| 166 | actgtgttccttg | 656 | AmCtgtgttccTTG | 0.081 |
| 168 | gaactgtgttcct | 657 | GAactgtgttmCmCT | 0.218 |
| 169 | agaactgtgttcc | 658 | AGaactgtgtTmCmC | 0.361 |
| 174 | gccagagaactgt | 659 | GmCcagagaacTGT | 0.007 |
| 175 | agccagagaactg | 660 | AGccagagaamCTG | 0.013 |
| 179 | gatgcatcagttt | 661 | GAtgcatcagTTT | 0.084 |
| 180 | ggatgcatcagtt | 662 | GGatgcatcaGTT | 0.052 |
| 181 | cggatgcatcagt | 663 | mCGgatgcatcAGT | 0.061 |
| 182 | tcggatgcatcag | 664 | TmCggatgcatmCAG | 0.072 |
| 183 | atcggatgcatca | 665 | ATcggatgcaTmCA | 0.054 |
| 189 | ggctttacatcgg | 666 | GGctttacatmCGG | 0.021 |
| 192 | agtgtgacatttt | 667 | AGtgtgacatTTT | 0.061 |
| 193 | aagtgtgacattt | 668 | AAgtgtgacaTTT | 0.035 |
| 197 | ggttccttggatg | 669 | GGttccttggATG | 0.036 |
| 198 | aggttccttggat | 670 | AGgttccttgGAT | 0.109 |
| 199 | taggttccttgga | 671 | TAggttccttGGA | 0.026 |
| 200 | ttaggttccttgg | 672 | TTaggttcctTGG | 0.010 |

TABLE 4

| SEQ | antisense | β2GPI relative |
|---|---|---|
| ID | oligonucleotide | expression |
| NO: | (5′→3′) | level |
| 673 | GGacatactcTGG | 0.070 |
| 674 | GGAcatactcTGG | 0.249 |
| 675 | AGGacatactcTGG | 0.069 |
| 676 | AAGgacatactcTGG | 0.141 |
| 677 | AAGgacatactmCTGG | 0.107 |
| 678 | AAGGacatactcTGG | 0.205 |
| 679 | AAGGacatactmCTGG | 0.219 |
| 680 | AAAGgacatactmCTGG | 0.121 |

### INDUSTRIAL APPLICABILITY

The present invention provides an antisense oligonucleotide having activity to suppress expression of β2GPI, a pharmaceutical composition comprising the antisense oligonucleotide as an active ingredient, and the like. The antisense oligonucleotide and pharmaceutical composition of the present invention suppress expression of β2GPI, and are useful for the prophylaxis or treatment of autoimmune diseases such as APS, SLE and the like and thrombosis in hemodialysis.

The contents disclosed in any publication stated in the present specification, including patents, patent applications and scientific literatures, are hereby incorporated in their entireties by reference, to the extent that they have been disclosed herein.

This application is based on a patent application No. 2015-140081 filed in Japan (filing date: Jul. 13, 2015), the contents of which are incorporated in full herein.

1. An antisense oligonucleotide consisting of 8-80 bases in length that suppresses an expression of β2GPI, comprising a sequence hybridizable to a nucleic acid consisting of a base sequence shown in any of SEQ ID NOs: 201-399 under stringent conditions.

2. An antisense oligonucleotide consisting of 8-80 bases in length that suppresses an expression of β2GPI, comprising at least 8 continuous bases in a base sequence shown in any of SEQ ID NOs: 2-200.

3. An antisense oligonucleotide consisting of 8-80 bases in length and complementary to a base sequence shown in any of SEQ ID NOs: 201-399.

4. The antisense oligonucleotide according to claim 3, comprising a base sequence shown in any of SEQ ID NOs: 2-200.

5. The antisense oligonucleotide according to claim 3, comprising a base sequence shown in any of SEQ ID NOs: 2-200 wherein 1 or several bases are deleted, substituted or added.

6. The antisense oligonucleotide according to claim 3, consisting of a base sequence shown in any of SEQ ID NOs: 2-200.

7. The antisense oligonucleotide according to claim 3, consisting of a base sequence shown in any of SEQ ID NOs: 400-680.

8. The antisense oligonucleotide according to claim 1, wherein the 5′-terminal vicinity and/or the 3′-terminal vicinity are/is constituted of a sugar moiety-modified nucleotide.

9. The antisense oligonucleotide according to claim 1, comprising a ligand.

10. A pharmaceutical composition comprising the antisense oligonucleotide according to claim 1.

11. The pharmaceutical composition according to claim 10 for the treatment or prophylaxis of an autoimmune disease or thrombosis.

12. A method of treating a disorder mediated by an anti-β2GPI antibody, comprising a step of administering a therapeutically effective amount of the antisense oligonucleotide according to claim 1 or the pharmaceutical composition comprising the antisense oligonucleotide according to claim 1 to a human in need of such treatment.

13. The method according to claim 12, wherein the aforementioned disorder is an autoimmune disease or thrombosis.

14-17. (canceled)
